(19) United States
(12) Patent Application Publication
(10) Pub. No.: US 2010/0160411 A1
(43) Pub. Date: Jun. 24, 2010

(54) BENZOXATHIINE AND BENZOXATHIOLE DERIVATIVES AND USES THEREOF

(75) Inventors: Gary Paul Stack, Ambler, PA (US); Ivana Bianchi, Fagnano Olona (IT); Stefania Vallese, Cardano al Campo (IT); Giovanna Luoni, Cardano al Campo (IT)

Correspondence Address: CHOATE, HALL & STEWART LLP/WYETH LLC, PATENT GROUP, TWO INTERNATIONAL PLACE, BOSTON, MA, 02110, US

(73) Assignee: Wyeth, Madison, NJ (US)

(21) Appl. No.: 12/446,904

(22) PCT Filed: Oct. 24, 2007
(86) PCT No.: PCT/US07/82425

§ 371 Date: Apr. 23, 2009

Related U.S. Application Data
(60) Provisional application No. 60/853,816, filed on Oct. 24, 2006.

(51) Int. Cl. A61K 31/39; C07D 327/06; C07D 327/04; A61P 25/18; A61P 25/00; A61P 15/00; A61P 13/10; A61P 25/24; A61P 25/16; A61P 25/08; A61P 25/22; A61P 25/30; A61P 25/06

(52) U.S. Cl. 514/434; 549/15; 514/439; 549/33; 549/32

(57) ABSTRACT

Compounds of formula (I) or pharmaceutically acceptable salts thereof are provided, wherein each of R¹, R², R³, R⁴, y, n, m, p, and Ar are as defined, and described in classes and subclasses herein, which are agonists or partial agonists of the 2C subtype of brain serotonin receptors. The compounds, and compositions containing the compounds, can be used to treat a variety of central nervous system disorders such as schizophrenia.

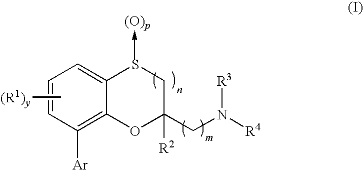

### CROSS-REFERENCE TO RELATED

This application claims priority to U.S. provisional patent application Ser. No. 60/853,816, filed Oct. 24, 2006, the entirety of which is hereby incorporated herein by reference.

### FIELD OF THE INVENTION

The present invention relates to 5-HT_{2C} receptor agonists or partial agonists, processes for their preparation, and uses thereof.

### BACKGROUND OF THE INVENTION

Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et. al., Am. J. Psychiatry, 156: 1686-1696, 1999; Masand, P. S., Exp. Opin. Pharmacother. I: 377-389, 2000; Whitaker, R., Spectrum Life Sciences. Decision Resources. 2:1-9, 2000).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology 124: 57-73, 1996; Cowen, P. J., et. al., Human Psychopharmacology 10: 385-391, 1995; Rosenzweig-Lipson, S., et. al., ASPET abstract, 2000).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et. al., Neuropharmacology 37: 265-272, 1998; Fox, S. H., et. al., Experimental Neurology 151: 35-49, 1998). Since the positive symptoms of schizophrenia are associated with increased levels of dopamine, compounds with actions opposite to those of 5-HT_{2C} antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology 37: 953-955, 1998; Di Matteo, V., et. al., Neuropharmacology 38: 1195-1205, 1999; Di Giovanni, G., et. al., Synapse 35: 53-61, 2000), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

### SUMMARY OF THE INVENTION

The present invention relates to 5-HT_{2C} receptor agonists or partial agonists and uses thereof. In one aspect, the invention relates to novel aryl substituted 2,3-dihydrobenzo[b][1,4]oxathiine and benzo[d][1,3]oxathiole derivatives that act as agonists or partial agonists of the 5-HT_{2C} receptor. The compounds can be used, for example, to treat schizophrenia and the concomitant mood disorders and cognitive impairments of schizophrenia and depression. In certain embodiments, compounds of the present invention are less likely to produce the body weight increases associated with current atypical antipsychotics. The compounds of the present invention can also be used for the treatment of obesity and its comorbidities. Compounds of the present invention are also useful for treating a variety of psychotic, depression and related disorders, and cognitive disorders as described in detail herein.

In certain embodiments, the present invention provides a compound of formula I:

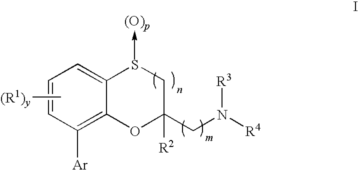

or a pharmaceutically acceptable salt thereof, wherein:
p is 0, 1, or 2;
m is 1 or 2;
n is 0 or 1;
Ar is phenyl, an 8-10 membered bicyclic partially unsaturated or aryl carbocyclic ring, a 5-6 membered monocyclic heteroaryl having 1-4 heteroatoms independently selected from nitrogen, oxygen, or sulfur, or an 8-10 membered bicyclic partially unsaturated or heteroaryl ring having 1-5 heteroatoms independently selected from nitrogen, oxygen, or sulfur, wherein Ar is optionally substituted with one or more R^{x} groups;
each R^{x} is independently selected from —R, —CN, halogen, —OR, —O(C₁₋₆ haloalkyl), —C(O)NH₂, —C(O)OR, C₁₋₆ haloalkyl, —NHC(O)R, —SO₂R, or —NHSO₂R;
y is 0-3;
each R¹ is independently —R, —CN, halogen, —OR, —O(C₁₋₆ haloalkyl), —C(O)NH₂, —C(O)OR, C₁₋₆ haloalkyl, —NHC(O)R, —SO₂R, or —NHSO₂R;
each R is independently hydrogen or C₁₋₆ aliphatic;
each of R², R³ and R⁴ is independently R or C₁₋₆ haloalkyl;

In certain other embodiments, the invention relates to methods for treating a patient suffering from schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, intellectual deficit associated with Alzheimer's disease, bipolar disorders, depressive disorders, mood episodes, anxiety disorders, adjustment disorders, eating disorders, epilepsy, sleep disorders, migraines, sexual dysfunction, substance abuse, addiction to alcohol and various other drugs, including cocaine and nicotine, gastrointestinal disorders, obesity, or a central nervous system deficiency associated with trauma, stroke, or spinal cord injury, or other conditions or disorders as described herein, that includes administering to the patient a therapeutically effective amount of a compound of formula I, or a pharmaceutically acceptable salt thereof.

In still other embodiments, the invention relates to compositions comprising a compound of formula I or a pharmaceutically acceptable salt thereof, and one or more pharmaceutically acceptable carriers, excipients, or diluents.

### DETAILED DESCRIPTION OF THE INVENTION

### 1. Compounds and Definitions

The present invention relates to novel aryl substituted 2,3-dihydrobenzo[b][1,4]oxathiine and benzo[d][1,3]oxathiole derivatives that are agonists or partial agonists of the 2C subtype of brain serotonin receptors.

The term “aliphatic” or “aliphatic group”, as used herein, means a straight-chain (i.e., unbranched) or branched, hydrocarbon chain that is completely saturated or that contains one or more units of unsaturation, or a monocyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. In certain embodiments, aliphatic groups contain 1-4 aliphatic carbon atoms, and in yet other embodiments, aliphatic groups contain 1-3 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Such cycloaliphatic groups include cycloalkyl and cycloalkenyl groups. Suitable aliphatic groups include, but are not limited to, linear or branched alkyl, alkenyl, alkynyl groups and hybrids thereof such as (cycloalkyl)alkyl, (cycloalkenyl)alkyl or (cycloalkyl)alkenyl.

The term “unsaturated,” as used herein, means that a moiety has one or more units of unsaturation.

The term “lower alkyl,” as used herein, refers to a hydrocarbon chain having up to 4 carbon atoms, preferably 1 to 3 carbon atoms, and more preferably 1 to 2 carbon atoms. The term “alkyl” includes, but is not limited to, straight and branched chains, e.g., of 1-6 carbon atoms, or 1-4 carbon atoms, such as methyl, ethyl, n-propyl, isopropyl, n-butyl, isobutyl, sec-butyl, or t-butyl.

The term “alkoxy,” as used herein, refers to the group —OR*, wherein R* is an alkyl, e.g., a lower alkyl group.

The terms “halogen” or “halo,” as used herein, refer to chlorine, bromine, fluorine or iodine.

The term “haloalkyl,” as used herein, or as part of a moiety such as “haloalkoxy” refers to an alkyl group, as defined herein, that has one or more halogen substituents. In certain embodiment, every hydrogen atom on said alkyl group is replaced by a halogen atom. Such haloalkyl groups include —CF₃. Such haloalkoxy groups include —OCF₃.

The term “alkenyl,” as used herein refers to an aliphatic straight or branched hydrocarbon chain having 2 to 4 carbon atoms that has one or more double bonds. Examples of alkenyl groups include vinyl, prop-1-enyl, allyl, methallyl, but-1-enyl, but-2-enyl, or but-3-enyl. The term “lower alkenyl” refers to an alkenyl group having up to 3 carbon atoms.

The term “aryl,” as used herein whether alone or as part of another group refers to a mono- or bicyclic aromatic ring system containing 6-10 carbon atoms where at least one of the rings of the bicyclic ring system is aromatic. Exemplary aryl groups include phenyl and naphthyl. In certain embodiments, the term “aryl,” as used herein refers to an 8-10 membered bicyclic partially unsaturated the wherein at least one of the rings is aromatic.

The term “heteroaryl,” as used herein, whether alone or as part of another group, refers to a mono- or bi-cyclic aromatic ring system containing 5-10 ring members of which 1-5 ring members are heteroatoms selected from N, O or S. At least one of the rings of the bicyclic ring system is heteroaromatic. For example ‘heteroaryl’ includes a 5-6 membered monocyclic heteroaryl having 1-4 heteroatoms independently selected from nitrogen, oxygen, or sulfur, or an 8-10 membered bicyclic partially unsaturated or heteroaryl ring having 1-5 heteroatoms independently selected from nitrogen, oxygen, or sulfur. Examples of heteroaryls include, but are not limited to, thienyl, furyl, pyrrolyl, imidazolyl, pyrazolyl, oxazolyl, isoxazolyl, thiazolyl, isothiazolyl, pyridyl, pyrazinyl, pyrimidinyl, pyridazinyl, indolyl, indazolyl, benzofuranyl, isobenzofuranyl, benzothienyl, isobenzothienyl, quinolyl, isoquinolyl, quinoxalinyl, or quinazolinyl.

The terms “effective amount” and “therapeutically effective amount,” as used herein, refer to the amount of a compound of formula I that, when administered to a patient, is effective to at least partially treat a condition from which the patient is suffering from. Such conditions include, but are not limited to, schizophrenia, schizoaffective disorder, schizophreniform disorder, L-DOPA-induced psychosis, bipolar disorder, obesity, obsessive compulsive disorder, depression, panic disorder, sleep disorders, eating disorders, and epilepsy.

The term “pharmaceutically acceptable salts” or “pharmaceutically acceptable salt” includes acid addition salts, that is salts derived from treating a compound of formula I with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. Where a compound of formula I contains a substituent with acidic properties, for instance, phenolic hydroxyl, —SO₂H or —CO₂H as R¹ or R^{x}, the term also includes salts derived from bases, for example, sodium salts.

The term “patient,” as used herein, refers to a mammal. In certain embodiments, the term “patient,” as used herein, refers to a human.

The terms “administer,” “administering,” or “administration,” as used herein, refer to either directly administering a compound or composition to a patient, or administering a prodrug derivative or analog of the compound to the patient, which will form an equivalent amount of the active compound or substance within the patient's body.

The terms “treat” or “treating,” as used herein, refers to partially or completely alleviating, inhibiting, preventing, ameliorating and/or relieving the condition.

The terms “suffer” or “suffering,” as used herein, refers to one or more conditions that a patient has been diagnosed with, or is suspected to have.

### 2. Description of Exemplary Compounds

In certain embodiments, the invention relates to a compound of formula I:

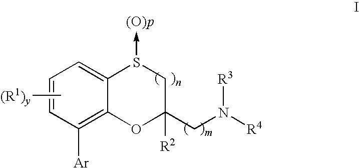

or a pharmaceutically acceptable salt thereof, wherein:
p is 0, 1, or 2;
m is 1 or 2;
n is 0 or 1;
Ar is phenyl, an 8-10 membered bicyclic partially unsaturated or aryl carbocyclic ring, a 5-6 membered monocyclic heteroaryl having 1-4 heteroatoms independently selected from nitrogen, oxygen, or sulfur, or an 8-10 membered bicyclic partially unsaturated or heteroaryl ring having 1-5 heteroatoms independently selected from nitrogen, oxygen, or sulfur, wherein Ar is optionally substituted with one or more R^{x} groups;
each R^{x} is independently selected from —R, —CN, halogen, —OR, —O(C₁₋₆ haloalkyl), —C(O)NH₂, —C(O)OR, C₁₋₆ haloalkyl, —NHC(O)R, —SO₂R, or —NHSO₂R;
y is 0-3;
each R¹ is independently —R, —CN, halogen, —OR, —O(C₁₋₆ haloalkyl), —C(O)NH₂, —C(O)OR, C₁₋₆ haloalkyl, —NHC(O)R, —SO₂R, or —NHSO₂R;
each R is independently hydrogen or C₁₋₆ aliphatic;
each of R², R³ and R⁴ is independently R or C₁₋₆ haloalkyl.

As defined generally above, the n group of formula I is 0 or 1. In certain embodiments, n is 1 thus forming a compound of formula Ia having a 2,3-dihydro-benzo[1,4]oxathiine ring:

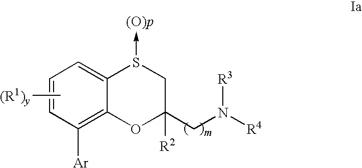

or a pharmaceutically acceptable salt thereof, wherein R¹, R², R³, R⁴, Ar, y, p, and m are as defined above for compounds of formula I and in classes and subclasses as described above and herein.

According to another embodiment, the n group of formula I is 0, thus forming a compound of formula Ib having a benzo[1,3]oxathiole ring:

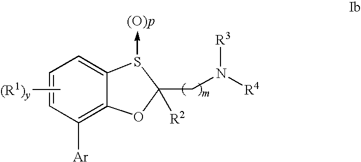

or a pharmaceutically acceptable salt thereof, wherein R¹, R², R³, R⁴, Ar, y, p, and m are as defined above for compounds of formula I and in classes and subclasses as described above and herein.

As defined generally above, y is 0-3 and each R¹ group of formula I is independently —R, —CN, halogen, —OR, —O(C₁₋₆ haloalkyl), —C(O)NH₂, —C(O)OR, C₁₋₆ haloalkyl, —NHC(O)R, —SO₂R, or —NHSO₂R. In certain embodiments, each R¹ group of formula I is independently —R, —CN, halogen, —OR, —OCF₃, or —CF₃. In other embodiments, each R¹ group of formula I is independently hydrogen, C₁₋₃ aliphatic, halogen, —OH, —O(C₁₋₃ aliphatic), —OCF₃ or —CF₃. In still other embodiments, y is 1, and R¹ is halogen.

According to one embodiment, y is 1, n is 1, and R¹ is at the 6- or 7-position of the benzoxathiine ring of formula I, thus forming a compound of formula IIa or IIb:

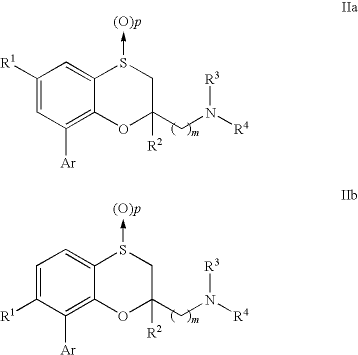

or a pharmaceutically acceptable salt thereof, wherein each R¹, R², R³, R⁴, Ar, p, and m are as defined above for compounds of formula I and in classes and subclasses as described above and herein.

According to another embodiment, y is 1, n is 0, and R¹ is at the 5- or 6-position of the benzoxathiole ring of formula I, thus forming a compound of formula IIc or IId:

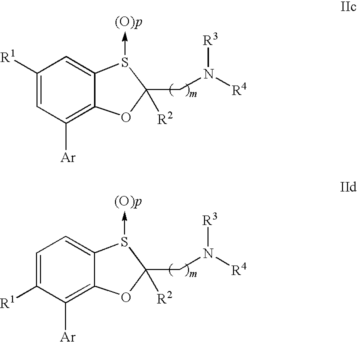

or a pharmaceutically acceptable salt thereof, wherein each R¹, R², R³, R⁴, Ar, p, and m are as defined above for compounds of formula I and in classes and subclasses as described above and herein.

As defined generally above, the Ar group of formula I is phenyl, an 8-10 membered bicyclic aryl ring, a 5-6 membered monocyclic heteroaryl having 1-4 heteroatoms independently selected from nitrogen, oxygen, or sulfur, or an 8-10 membered bicyclic heteroaryl ring having 1-5 heteroatoms independently selected from nitrogen, oxygen, or sulfur, wherein Ar is optionally substituted with one or more R^{x} groups, and wherein each R^{x} is independently selected from —R, —CN, halogen, —OR, —OCF₃, —C(O)NH₂, —C(O)OR, —CF₃, —NHC(O)R, —SO₂R, or —NHSO₂R. In certain embodiments, the Ar group of formula I is phenyl, an 8-10 membered bicyclic aryl ring, or a 5-6 membered monocyclic heteroaryl having 1-4 heteroatoms independently selected from nitrogen, oxygen, or sulfur, wherein Ar is optionally substituted with R^{x}. In other embodiments, the Ar group of formula I is pyridyl, pyrimidinyl, thienyl, or furanyl, wherein Ar is optionally substituted with R^{x}. In still other embodiments, the Ar group of formula I is phenyl, optionally substituted with one or more R^{x} groups. According to one embodiment, Ar is phenyl substituted with R^{x} in the ortho-position thus forming a compound of formula IIIa or IIIb:

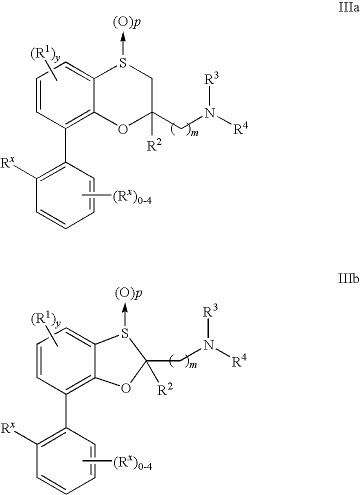

or a pharmaceutically acceptable salt thereof, wherein each R¹, R², R³, R⁴, R^{x}, y, p, and m are as defined above for compounds of formula I and in classes and subclasses as described above and herein.

According to another embodiment, the Ar group of formula I is phenyl substituted with an R^{x} group in both ortho-positions thus forming a compound of formula IIIc or IIId:

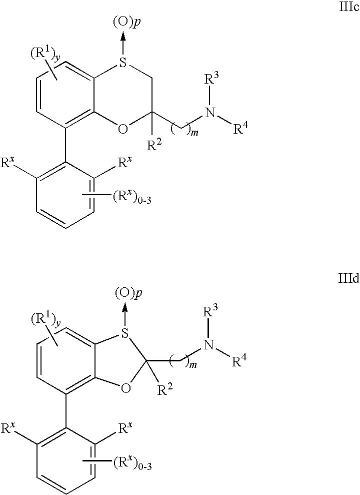

or a pharmaceutically acceptable salt thereof, wherein each R¹, R², R³, R⁴, R^{x}, y, p, and m are as defined above for compounds of formula I and in classes and subclasses as described above and herein.

According to one aspect of the present invention, the Ar group of formula I is substituted with one or more R^{x} groups independently selected from —R, —CN, halogen, —OR, OCF₃, or —CF₃. According to another aspect of the present invention, the Ar group of formula I is substituted with one or two R^{x} groups independently selected from halogen, —OR, —R, or —CF₃.

In certain embodiments, the Ar group of at least one of I, Ia, Ib, IIa, IIb, IIc, IId, IIIa, IIIb, IIIc, and IIId is selected from the following:

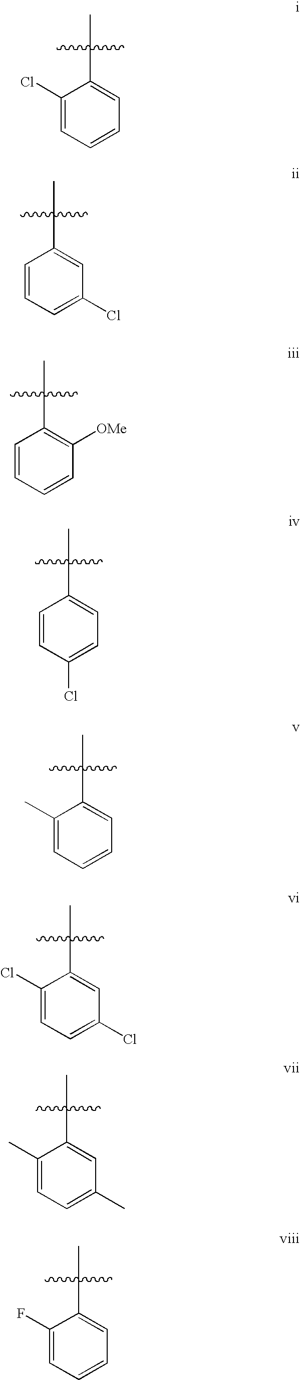

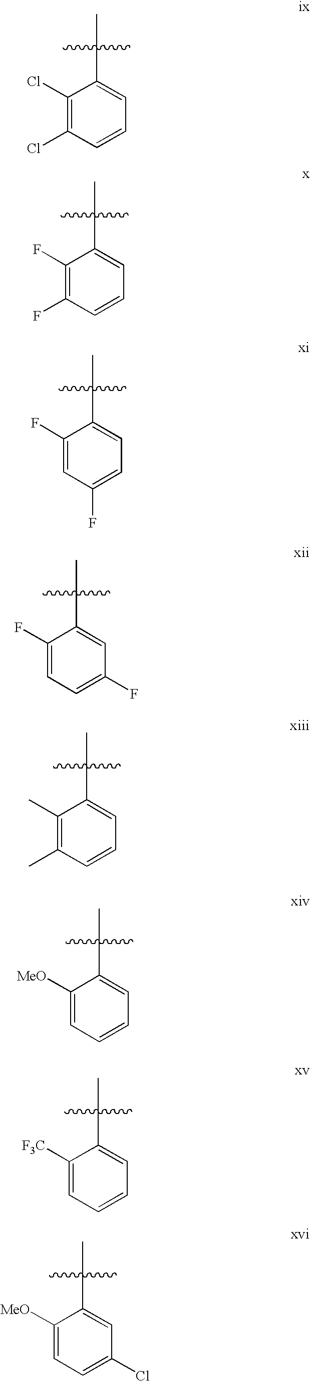

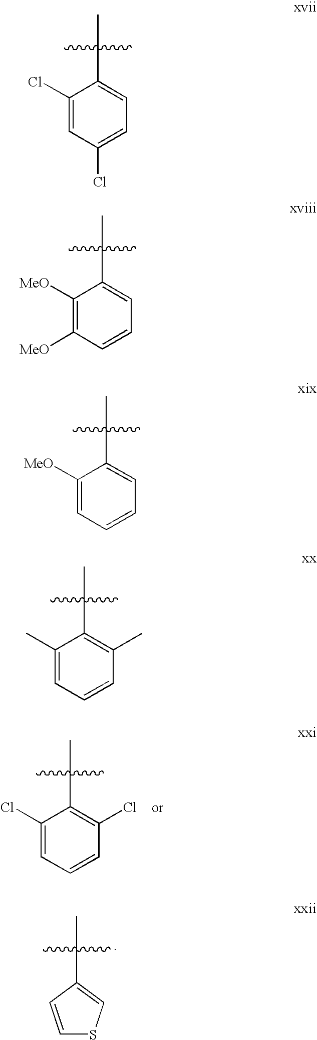

In other embodiments, the Ar group of at least one of formulae I, Ia, Ib, IIa, IIb, IIc, IId, IIIa, IIIb, IIIc, and IIId is selected from the following:

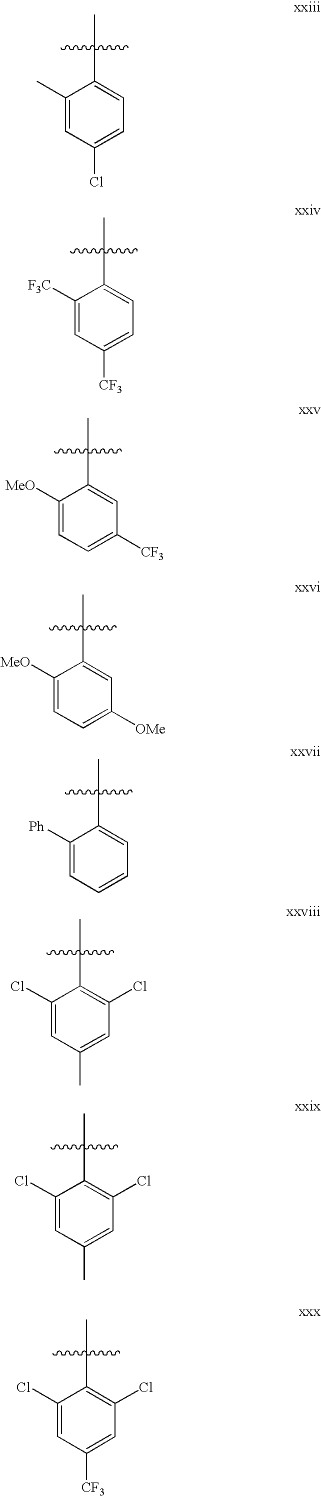

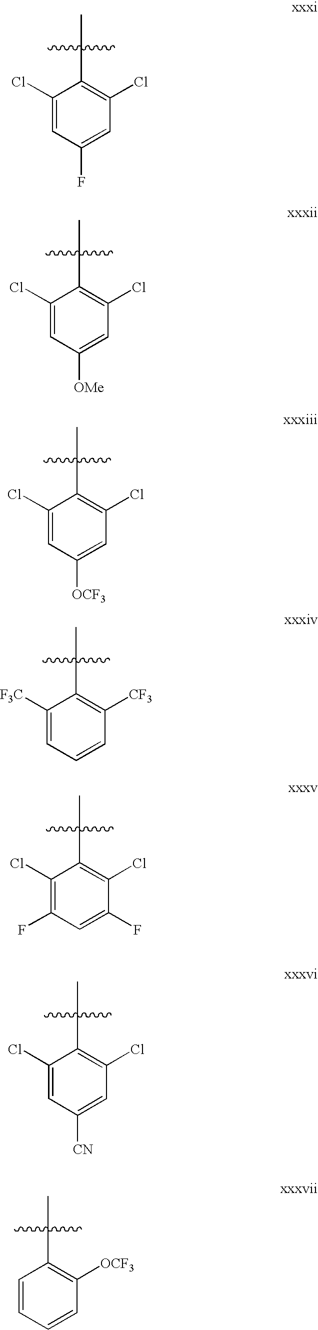

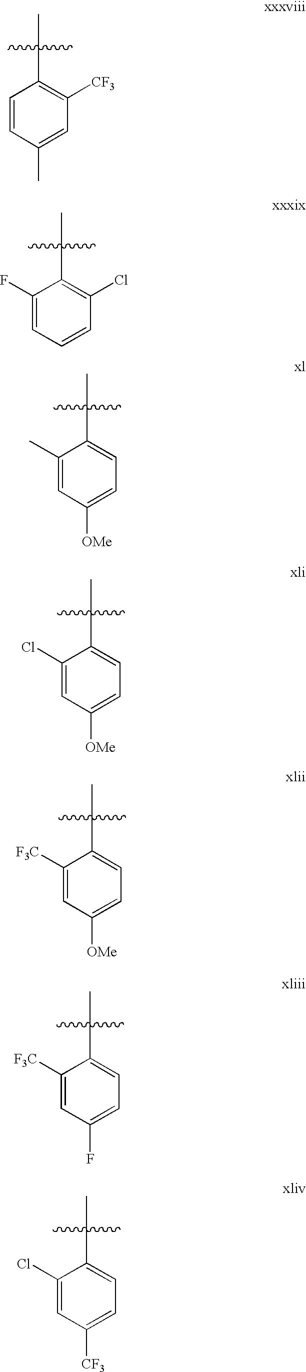

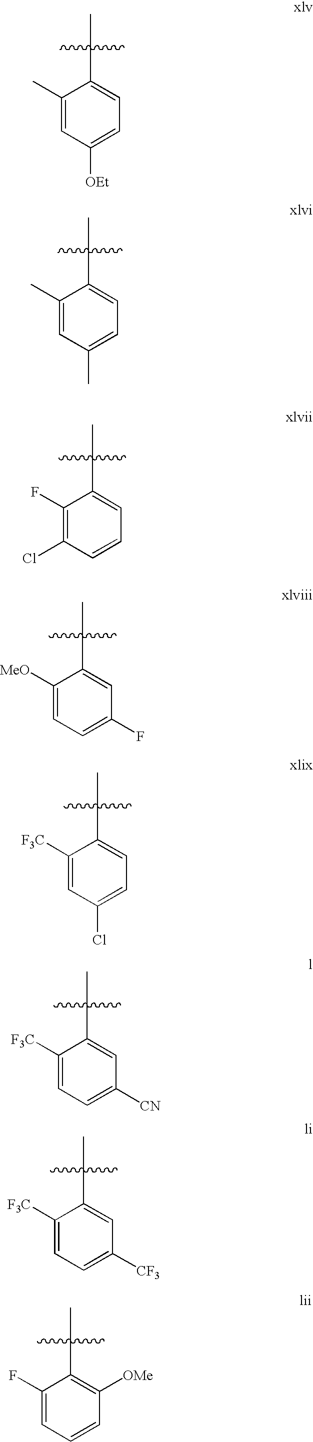

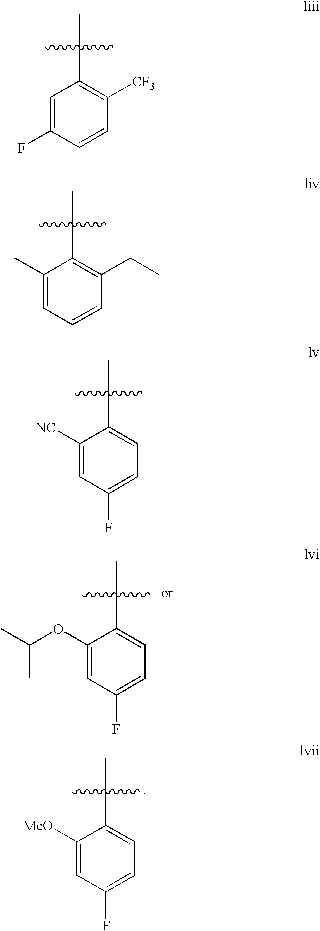

As defined generally above, the R² of formula I is R or C₁₋₆ haloalkyl. In certain embodiments, the R² of formula I is hydrogen, methyl, or —CF₃. In other embodiments, the R² of formula I is hydrogen or methyl.

As defined generally above, the R³ and R⁴ groups of formula I are each independently hydrogen or C₁₋₆ aliphatic. In certain embodiments, both of the R³ and R⁴ groups of formula I are hydrogen. In other embodiments, neither of the R³ or R⁴ groups of formula I is hydrogen. According to one aspect of the present invention, the R³ and R⁴ groups of formula I are independently hydrogen, methyl, ethyl, cyclopropyl, cyclopropylmethyl, n-propyl, allyl, or cyclobutyl. Yet another aspect of the present invention provides a compound of formula I wherein one of the R³ and R⁴ groups of formula I is hydrogen and the other is methyl, ethyl, cyclopropyl, cyclopropylmethyl, n-propyl, allyl, or cyclobutyl.

Compounds of the present invention contain asymmetric carbon atoms and thus give rise to stereoisomers, including enantiomers and diastereomers. Accordingly, it is contemplated that the present invention relates to all of these stereoisomers, as well as to mixtures of the stereoisomers. Throughout this application, the name of the product of this invention, where the absolute configuration of an asymmetric center is not indicated, is intended to embrace the individual stereoisomers as well as mixtures of stereoisomers.

In certain embodiments, the present invention provides a compound of formula IVa, IVb, IVc, or IVd:

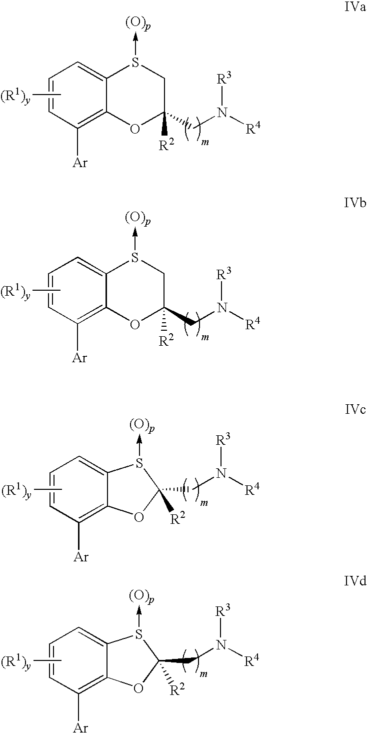

or a pharmaceutically acceptable salt thereof, wherein each R¹, R², R³, R⁴, Ar, y, p, and m are as defined above for compounds of formula I and in classes and subclasses as described above and herein.

According to another embodiment, the present invention provides a compound of formula Va, Vb, Vc, or Vd:

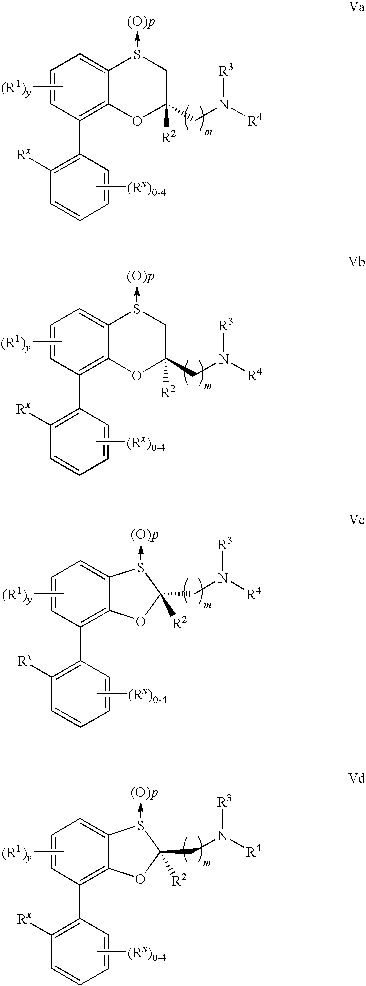

or a pharmaceutically acceptable salt thereof, wherein each R¹, R², R³, R⁴, R^{x}, y, p, and m are as defined above for compounds of formula I and in classes and subclasses as described above and herein.

In other embodiments, the present invention provides a compound of formula VIa, VIb, VIc, or VId:

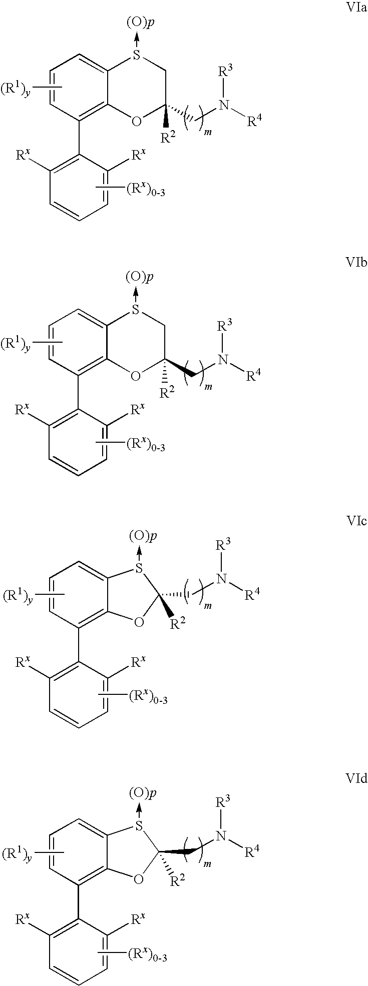

or a pharmaceutically acceptable salt thereof, wherein each R¹, R², R³, R⁴, R^{x}, y, p, and m are as defined above for compounds of formula I and in classes and subclasses as described above and herein.

According to another embodiment, the present invention provides a compound of formula VIIa, VIIb, VIIc, or VIId:

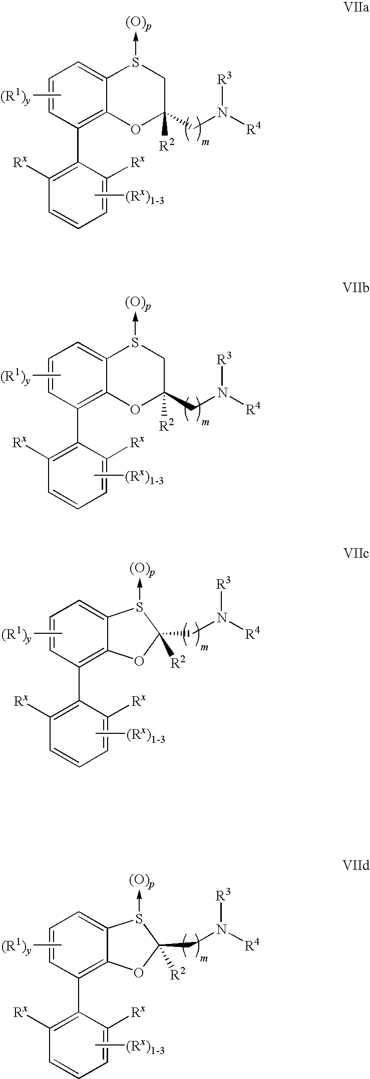

or a pharmaceutically acceptable salt thereof, wherein each R¹, R², R³, R⁴, R^{x}, y, p, and m are as defined above for compounds of formula I and in classes and subclasses as described above and herein, and wherein R^{x} is not H.

Where an enantiomer is preferred, it may, in some embodiments be provided substantially free of the corresponding enantiomer. Thus, an enantiomer substantially free of the corresponding enantiomer refers to a compound which is isolated or separated via separation techniques or prepared free of the corresponding enantiomer. “Substantially free,” as used herein, means that the compound is made up of a significantly greater proportion of one enantiomer. In certain embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including high pressure liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N.Y., 1962); Wilen, S. H. Tables of Resolving Agents and Optical Resolutions p. 268 (E. L. Eliel, Ed., Univ. of Notre Dame Press, Notre Dame, Ind. 1972).

It is further recognized that atropisomers of the present compounds may exit. The present invention thus encompasses atropisomeric forms of compounds of formula I as defined above, and in classes and subcasses described above and herein.

Exemplary compounds of formula I are set forth in Table 1, below.

TABLE 1
Exemplary Compounds of Formula I:

C-[8-(2,6-Dichloro-phenyl)-6-fluoro-2,3-dihydro-benzo[1,4]oxathiin-2-yl]-methylamine;
C-[8-(2,6-Dichloro-phenyl)-6-fluoro-4,4-dioxo-3,4-dihydro-2H-4lambda-6-
benzo[1,4]oxathiin-2-yl]-methylamine hydrochloride salt;
C-[8-(2,6-Dichloro-phenyl)-6-fluoro-4-oxo-3,4-dihydro-2H-4lambda-4-benzo[1,4]oxathiin-2-
yl]-methylamine;
1-[7-(2,6-dichlorophenyl)-5-fluoro-1,3-benzoxathiol-2-yl]methanamine;
1-{6-fluoro-8-[2-(trifluoromethyl)phenyl]-2,3-dihydro-1,4-benzoxathiin-2-
yl}methanamine;
1-[7-(2,6-dichlorophenyl)-5-fluoro-3,3-dioxido-1,3-benzoxathiol-2-yl]methanamine;
1-{5-fluoro-7-[2-(trifluoromethyl)phenyl]-1,3-benzoxathiol-2-yl}methanamine; and
1-[8-(2-chlorophenyl)-6-fluoro-2,3-dihydro-1,4-benzoxathiin-2-yl]methanamine,
or a pharmaceutically acceptable salt, enantiomer, or racemate thereof.

As defined above, the p group of formula I is 0, 1, or 2. Accordingly, compounds of the present invention are contemplated wherein the sulfur atom is not oxidized or is oxidized to S(O) or S(O)₂. Although such oxidized forms are contemplated for all compounds of the present invention, these forms are exemplified for formula I below:

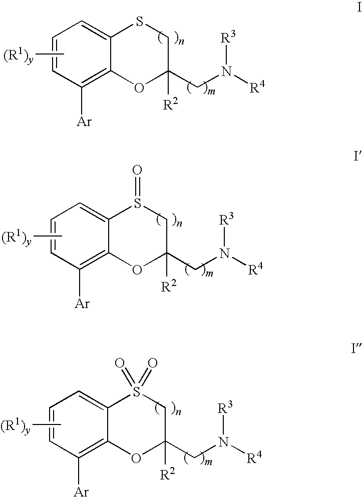

or a pharmaceutically acceptable salt thereof, wherein each R¹, R², R³, R⁴, y, n, and m are as defined above for compounds of formula I and in classes and subclasses as described above and herein.

### 3. General Methods of Providing the Present Compounds

Compounds of formula I of the present invention are prepared as illustrated in Schemes 1-4, below. Unless otherwise noted, all variables are as defined above and in classes and subclasses described above and herein.

The benzoxathiine derivatives (Ia) of the present invention are prepared as illustrated in Scheme 1, below. The Suzuki coupling of the appropriately substituted o-methoxyphenylboronic acid and aryl bromide or triflate in the presence of a base such as potassium carbonate and a suitable catalyst such as tetrakis(triphenylphosphine)palladium (0) affords the biphenyl derivative A. The biphenyl compound A is iodinated ortho to the methoxy group by treatment with N-iodosuccinimide and sulfuric acid or brominated with N-bromosuccinimide in dioxane to produce intermediate B, which after successive treatment with n-butyl lithium and dimethyl disulfide in THF provides the thiomethyl derivative C. Selective demethylation of the ether is achieved via treatment with boron tribromide in dichloromethane to give D. Heating D in epibromohydrin gives the benzoxathiine E. The alcohol E is converted to tosylate F by treatment with p-toluenesulfonyl chloride and triethylamine in methylene chloride. Conversion of the tosylate to the azide and subsequent reduction to the amine with a suitable reducing agent such as polymer-supported triphenylphosphine in tetrahydrofuran and water gives the title compounds of formula (Ia) of the present invention in which p is 0 and n is 1.

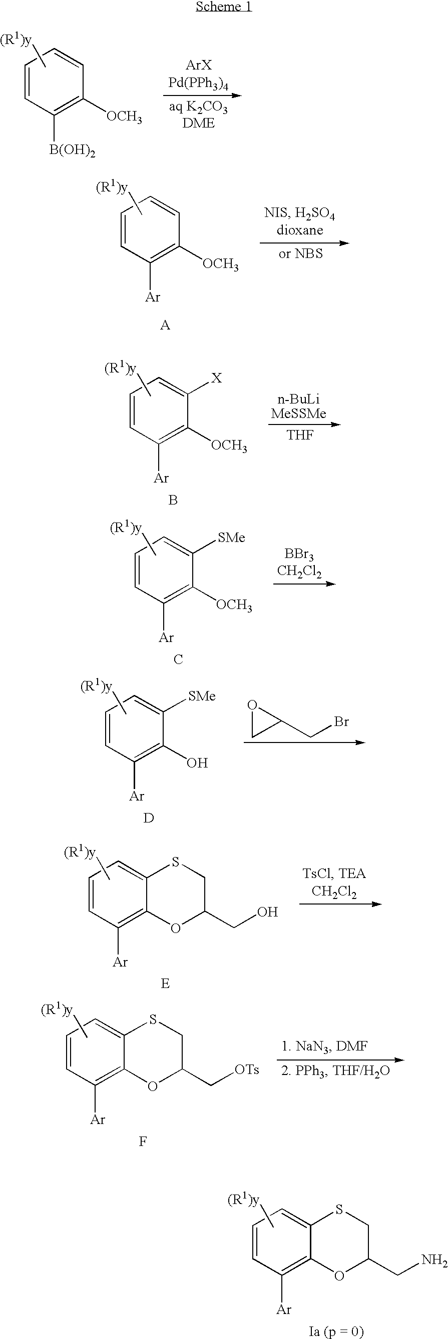

Scheme 2, below, depicts a method for preparing compounds of formula Ia in which p and n are 1. The intermediate tosylate F from Scheme 1 is treated with potassium phthalimide in DMF to give the phthalimido derivative G. Oxidation with m-chloroperoxybenzoic acid in methylene chloride at −78° C. gives the sulfoxide H. Removal of the phthalimido protecting group by treatment with hydrazine in a suitable solvent such as ethanol affords the title compounds of formula (Ia) of the invention.

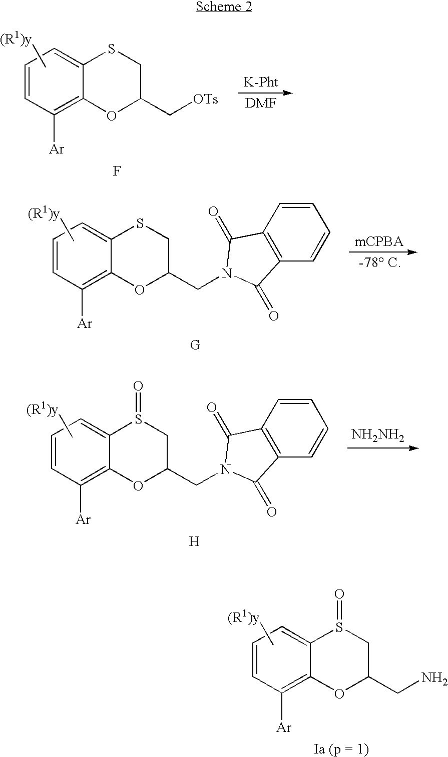

Alternatively, the sulfur of the benzoxathiine ring in compound I can be oxidized to sulfur VI via the procedure outlined in Scheme 3 below. Compound Ia (p=0) from Scheme 1 is first protected as the t-Boc derivative by treatment with t-Boc anhydride and a suitable base such as triethylamine to give intermediate J. The t-Boc protected intermediate J is oxidized to the sulfone by treatment with m-chloroperoxybenzoic acid at room temperature to give intermediate K, which when deprotected by treatment with HCl in ether affords the compounds of the invention in which n is 1 and p is 2.

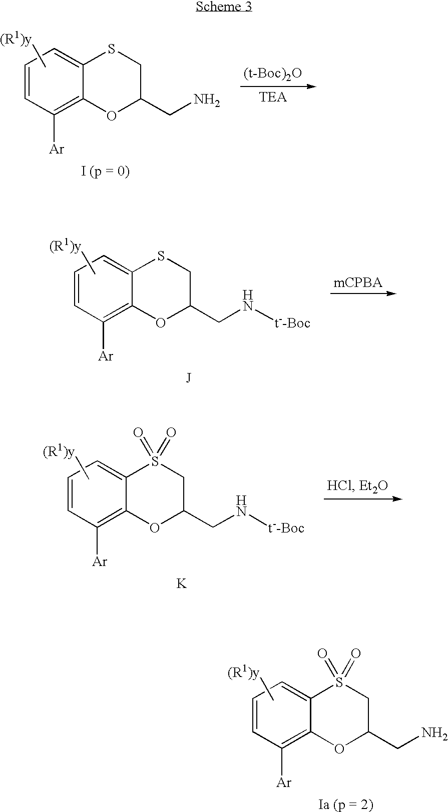

Compounds of the invention in which n is 0 are prepared according to the procedure outlined in Scheme 4 below. Intermediate D from Scheme 1 above is treated in a microwave at 140° C. with diethyl dibromomalonate in the presence of potassium carbonate and 4 Å molecular sieves to yield the benzoxathiole dicarboxylate L. Hydrolysis and decarboxylation is effected by treatment with 20% HCl in acetic acid at 100° C. for 6 hours to give M. The acid M is reduced with borane THF, converted to tosylate P by treatment with p-toluenesulfonyl chloride and a suitable base such as triethylamine and treated with sodium azide in DMF to afford the azide derivative Q. As before, azide reduction with polymer-supported triphenylphosphine in THF/water gives the title compounds of formula Ib of the invention.

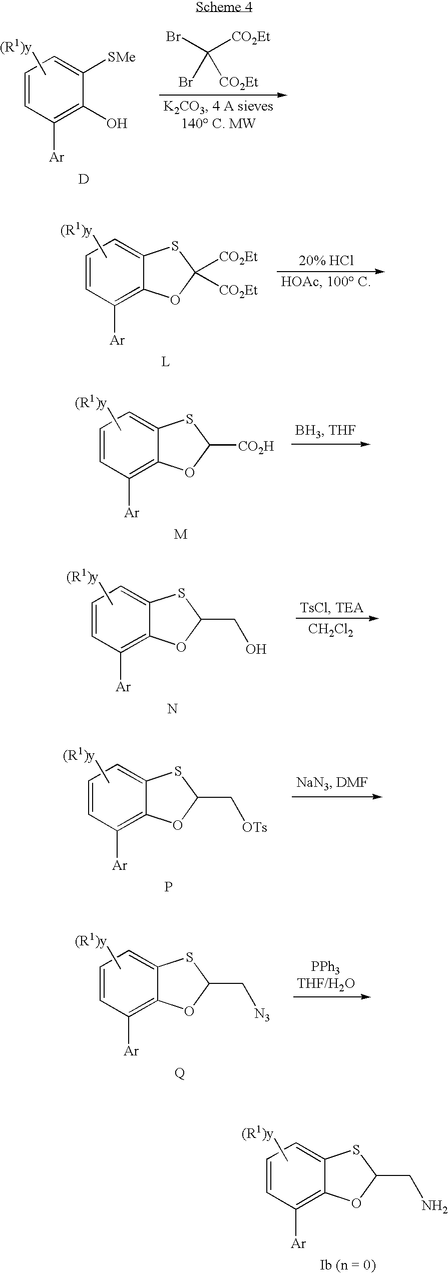

One of ordinary skill in the art will recognize that compounds of the invention in which either R³ and R⁴ is alkyl can be prepared by substituting the appropriate primary or secondary amine for sodium azide in the above schemes.

Although certain exemplary embodiments are depicted and described above and herein, it will be appreciated that compounds of the invention can be prepared according to the methods described generally above using appropriate starting materials by methods generally available to one of ordinary skill in the art. Additional embodiments are exemplified in more detail herein.

### 4. Uses, Formulation and Administration

Compounds of the present invention have affinity for and agonist or partial agonist activity at the 2C subtype of brain serotonin receptors and are thus of interest for the treatment of a variety of disorders and/or the alleviation of one or more associated symptoms. Such disorders associated with modulations of the 2C subtype of brain serotonin receptors are described in detail below. The present invention contemplates that compounds of formula I are associated with a rapid onset of action. In addition, compounds of formula I lack the side-effect of sexual dysfunction.

Compounds of the present invention are useful for treating one or more psychotic disorders, as described herein, without causing diabetogenesis. Diabetogenesis is a side-effect associated with atypical antipsychotic agents. Without wishing to be bound by any particular theory, it is believed that the diabetogenesis associated with atypical antipsychotic agents results from the fact that those agents are 5-HT_{2C} antagonists. As described herein, the present compounds are 5-HT_{2C} agonists, or partial agonists, and therefore are not associated with diabetogenesis.

Compounds of the present invention are useful for treating one or more psychotic disorders such as schizophrenia including paranoid type, disorganized type, catatonic type, and undifferentiated type, schizophreniform disorder, schizoaffective disorder, delusional disorder, substance-induced psychotic disorder, and psychotic disorder not otherwise specified; L-DOPA-induced psychosis; psychosis associated with Alzheimer's dementia; psychosis associated with Parkinson's disease; and psychosis associated with Lewy body disease.

Compounds of the present invention are also useful for treating symptoms related to psychotic disorders of the schizophrenic types, including the so called “positive” and “negative” symptoms of schizophrenia. These symptoms include for example hallucinations, delusions, paranoia, anxiety, agitation, excessive aggression, tension, thought disorder, blunted affect, and social or emotional withdrawal in psychotic patients. Other symptoms often associated with psychotic disorders include cognition disorders or deficits such as poor attention and impaired function, depression, suicide, metabolic syndrome, and substance abuse. Thus, another embodiment of the present invention provides a method for treating one or more symptoms associated with a psychotic disorder.

In other embodiments, the present compounds are useful for treating anxiety disorders such as panic attack, agoraphobia, panic disorder, specific phobia, social phobia, social anxiety disorder, obsessive compulsive disorder, posttraumatic stress disorder, acute stress disorder, generalized anxiety disorder, separation anxiety disorder, substance-induced anxiety disorder, and anxiety disorder not otherwise specified.

According to another embodiment, the present compounds are useful for treating bipolar disorders. Such bipolar disorders include bipolar I disorder, bipolar II disorder, and cyclothymic disorder; bipolar mania, dementia, and depression with psychotic features. The present compounds are also useful for treating (including the preventing) of cycling that may occur between bipolar depression and bipolar mania.

A more complete description of the aforementioned mental disorders can be found in the Diagnostic and Statistical Manual of Mental Disorders, 4^{th} edition, Washington, D.C., American Psychiatric Association (1994), incorporated herein by reference in its entirety.

In certain embodiments, compounds of the present invention are administered in combination with one or more anti-psychotic agents. Such anti-psychotic agents are well known in the art and include clozapine (e.g., Clozaril®), risperidone (e.g., Risperidal®), olanzapine (e.g., Zyprexa®), quetiapine (e.g., Seroquel®), ziprasidone (e.g., Geodon®), aripiprazole, amisulpiride, chlorpromazine, fluphenazine, haloperidol (e.g., Haldol®), loxapine, mesoridazine, molindone, perphenazine, pimozide, seroquel, sulpiride, thioridazine, thiothixene, trifluoperazine, and bifeprunox to name a few.

The combination of a compound of the present invention with one or more anti-psychotic agents is useful for treating schizophrenia including paranoid type, disorganized type, catatonic type, and undifferentiated type, schizophreniform disorder, schizoaffective disorder, delusional disorder, substance-induced psychotic disorder, and psychotic disorder not otherwise specified; L-DOPA-induced psychosis; psychosis associated with Alzheimer's dementia; psychosis associated with Parkinson's disease; psychosis associated with Lewy body disease; bipolar disorders such as bipolar I disorder, bipolar II disorder, and cyclothymic disorder; bipolar mania, dementia, and depression with psychotic features. In some embodiments, these combinations are useful in the treatment of bipolar disorder, including for example treating the cycling between bipolar depression and bipolar mania.

In other embodiments, administration of a compound of the present invention with an anti-psychotic agent provide anti-psychotic benefits while eliminating or minimizing certain side affects (e.g., akathisia, dystonia, Parkinsonism dyskinesia and late dyskinesia and the like) typically observed when the anti-psychotic agent(s) is/are taken alone.

In other embodiments, compounds of the present invention are useful for treating one or more depressive disorders such as major depressive disorder, seasonal affective disorder, dysthymic disorder, substance-induced mood disorder, depressive disorder not otherwise specified, and treatment resistant depression.

Another aspect of the present invention provides a method for treating one or more mood episodes such as major depressive episode, manic episode, mixed episode, and hypomanic episode; and adjustment disorders such as adjustment disorders with anxiety and/or depressed mood.

Compounds of the present invention are also useful for treating symptoms related to depressive disorders including somatic symptoms such as neuropathic pain and sexual dysfunction. Other somatic symptoms include hopelessness, helplessness, anxiety and worries, memory complaints with or without objective signs of cognitive impairment, loss of feeling of pleasure (anhedonia), slowed movement, irritability, and lack of interest in personal care, such as poor adherence to medical or dietary regimens.

In certain embodiments, the present invention provides a method of treating sexual dysfunction related to depression. In other embodiments, the present invention provides a method of treating sexual dysfunction associated with administering a serotonin reuptake inhibitor (SRI) for treating a depressive or other disorder. Such methods of treating sexual dysfunction are described in detail below.

In certain embodiments, compounds of the present invention are administered in combination with one or more antidepressive agents. Suitable antidepressant agents include, for example, serotonin reuptake inhibitors (SRIs), norepinephrine reuptake inhibitors (NRIs), combined serotonin-norepinephrine reuptake inhibitors (SNRIs), monoamine oxidase inhibitors (MAOIs), reversible inhibitors of monoamine oxidase (RIMAs), phosphodiesterase-4 (PDE4) inhibitors, corticotropin releasing factor (CRF) antagonists, alpha-adrenoreceptor antagonists or other compounds including atypical antidepressants. Additional antidepressants for administering in combination with compounds of the present invention include triple uptake inhibitors such as DOV 216303 and DOV 21947; melatonin agonists such as agomelotine, super neurotransmitter uptake blockers (SNUBs; e.g., NS-2389 from GlaxoSmithKline and Neurosearch; (R)-DDMA from Sepracor), and/or substance P/neurokinin receptor antagonists (e.g., aprepitant/MK-869 from Merck; NKP-608 from Novartis; CPI-122721 from Pfizer; R673 from Roche; TAK637 from Takeda; and GW-97599 from GlaxoSmithKline).

Another class of antidepressant agents for administering in combination with compounds of the present invention is noradrenergic and specific serotonergic antidepressants (NaSSAs). A suitable example of a NaSSA is mirtazepine.

Suitable NRIs for administering in combination with compounds of the present invention include tertiary amine tricyclics and secondary amine tricyclics. Suitable examples of tertiary amine tricyclics include: amitriptyline, clomipramine, doxepin, imipramine (See U.S. Pat. No. 2,554,736, incorporated herein by reference in its entirety) and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

Another NRI for administering in combination with compounds of the present invention is reboxetine (Edronax™; 2-[.alpha.-(2-ethoxy)phenoxy-benzyl]morpholine, usually administered as the racemate; See U.S. Pat. No. 4,229,449, incorporated herein by reference in its entirety).

Suitable SSRIs for administering in combination with compounds of the present invention include: citalopram (1-[3-(dimethylamino)propyl]-(4-fluorophenyl)-1,3-dihydro-o-5-isobenzofurancarbonitrile; See U.S. Pat. No. 4,136,193; Christensen et al., Eur. J. Pharmacol. 41:153, 1977; Dufour et al., Int. Clin. Psychopharmacol. 2:225, 1987; Timmerman et al., ibid., 239, each of which is incorporated herein by reference in its entirety); fluoxetine (N-methyl-3-(p-trifluoromethylphenoxy)-3-phenylpropylamine, marketed in the hydrochloride salt form and as the racemic mixture of its two isoforms; see, for example, U.S. Pat. No. 4,314,081; Robertson et al., J. Med. Chem. 31:1412, 1988, each of which is incorporated herein by reference); fluoxetine/olanzapine in combination; fluvoxamine (5-methoxy-1-[4-(trifluoromethyl)phenyl]-1-pentanone O-(2-aminoethyl)oxime; See U.S. Pat. No. 4,085,225; Claassen et al., Brit. J. Pharmacol. 60:505, 1977; De Wilde et al., J. Affective Disord. 4:249, 1982; Benfield et al., Drugs 32:313, 1986, each of which is incorporated herein by reference in its entirety); paroxetine (trans-(−)-3-[(1,3-benzodioxol-5-yloxy)methyl]-4-(4-fluorophenyl)piperidine; See U.S. Pat. No. 3,912,743; U.S. Pat. No. 4,007,196; Lassen, Eur. J. Pharmacol. 47:351, 1978; Hassan et al., Brit. J. Clin. Pharmacol. 19:705, 1985; Laursen et al., Acta Psychiat. Scand. 71:249, 1985; Battegay et al., Neuropsychobiology 13:31, 1985, each of which is incorporated herein by reference in its entirety); sertraline, (1S-cis)-4-(3,4-dichlorophenyl)-1,2,3,4-tetrahydro-N-methyl-1-naphthylamine hydrochloride; See U.S. Pat. No. 4,536,518, incorporated herein by reference in its entirety); escitalopram (see U.S. Pat. RE34,712); and pharmaceutically acceptable salts thereof.

Suitable MAOIs for administering in combination with compounds of the present invention include: isocarboxazid, phenelzine, selegiline and tranylcypromine, and pharmaceutically acceptable salts thereof.

Suitable reversible MAOIs for administering in combination with compounds of the present invention include: moclobemide (4-chloro-N-[2-(4-morpholinyl)-ethyl]benzamide; See U.S. Pat. No. 4,210,754, incorporated herein by reference in its entirety), selegiline, and pharmaceutically acceptable salts thereof.

Suitable SNRIs for administering in combination with compounds of the present invention include venlafaxine (see U.S. Pat. No. 4,535,186, incorporated herein by reference in its entirety; see also U.S. Pat. Nos. 5,916,923, 6,274,171, 6,403,120, 6,419,958, 6,444,708, each of which is incorporated herein by reference in its entirety), and pharmaceutically acceptable salts and analogs, including the O-desmethylvenlafaxine succinate salt; milnacipran (N,N-diethyl-2-aminomethyl-1-phenylcyclopropanecarboxamide; see U.S. Pat. No. 4,478,836; Moret et al., Neuropharmacology 24:1211-19, 1985, each of which is incorporated herein by reference in its entirety); mirtazapine (see, for example, U.S. Pat. No. 5,178,878, the entire contents of which are incorporated herein by reference); nefazodone (available from Bristol Myers Squibb and Dr. Reddy Labs Inc.); duloxetine; and pharmaceutically acceptable salts thereof.

Suitable CRF antagonists for administering in combination with compounds of the present invention include those compounds described in International Patent Specification Nos. WO 94/13643, WO 94/13644, WO 94/13661, WO 94/13676 and WO 94/13677.

Suitable atypical antidepressants for administering in combination with compounds of the present invention include: bupropion (Wellbutrin™; (.+−−.)-1-(3-chlorophenyl)-2-[(1,1-dim-ethylethyl)amino]-1-propanone), lithium, nefazodone, trazodone and viloxazine, and pharmaceutically acceptable salts thereof. Another suitable atypical antidepressant is sibutramine.

Particular antidepressants for administering in combination with compounds of the present invention include, but are not limited to, adinazolam, alaproclate, alnespirone, amineptine, amitriptyline, amitriptyline/chlordiazepoxide combination, amoxapine, aprepitant, atipamezole, azamianserin, bazinaprine, befuraline, bifemelane, binodaline, bipenamol, brofaromine, buproprion, caroxazone, cericlamine, cianopramine, cimoxatone, citalopram, clemeprol, clomipramine, clovoxamine, dazepinil, deanol, demexiptiline, desipramine, O-desmethylvenlafaxine, dibenzepin, dothiepin, doxepin, droxidopa, duloxetine, elzasonan, enefexine, eptapirone, escitalopram, estazolam, etoperidone, femoxetine, fengabine, fezolamine, fluotracen, fluoxetine, fluvoxamine, gepirone, idazoxan, imipramine, indalpine, indeloxazine, iprindole, isocarboxazid, levoprotiline, litoxetine, lofepramine, maprotiline, medifoxamine, metapramine, metralindole, mianserin, milnacipran, minaprine, mirtazapine, moclobemide, montirelin, nebracetam, nefopam, nefozodine, nemititide, nialamide, nomifensine, norfluoxetine, nortriptyline, orotirelin, oxaflozane, paroxetine, pheneizine, pinazepam, pirlindone, pizotyline, protryptiline, reboxetine, ritanserin, robalzotan, rolipram, selegiline, sercloremine, sertraline, setiptiline, sibutramine, sulbutiamine, sulpiride, sunepitron, teniloxazine, thozalinone, thymoliberin, tianeptine, tiflucarbine, tofenacin, tofisopam, toloxatone, tomoxetine, tranylcypromine, trazodone, trimiprimine, venlafaxine, veralipride, vilazodone, viloxazine, viqualine, zimelidine and zometrapine, and pharmaceutically acceptable salts thereof, and St. John's wort herb, or Hypencuin perforatum, or extracts thereof.

Suitable classes of anti-anxiety agents for administering in combination with compounds of the present invention include 5-HT_{IA} agonists or antagonists, especially 5-HT_{IA} partial agonists, neurokinin recepter (NK) antagonists (e.g., saredutant and osanetant) and corticotropin releasing factor (CRF) antagonists. Suitable 5-HT_{1A} receptor agonists or antagonists that may be used in the present invention include, in particular, the 5-HT_{IA} receptor partial agonists buspirone, flesinoxan, gepirone and ipsapirone, and pharmaceutically acceptable salts thereof. An example of a compound with 5-HT_{IA} receptor antagonist/partial agonist activity is pindolol. New 5HT_{1A} agonists variza, alnespirone, gepirone, sunepitron, MKC242, vilazodone, eptapirone, and ORG12962 from Organon; new 5HT_{1A} antagonists such as robalzotan; new 5-HT_{1B} agonists such as elzasonan; new 5HT₂ antagonists such as YM-992 (from Yamanouchi Pharmaceuticals) and nemifitide.

According to the present invention, the inventive combinations may be administered in conjunction with one or more other agents that is useful in treating depression or other mood disorders. Alternatively or additionally, inventive combinations may be administered with one or more other pharmaceutical agents active in treating any other symptom or medical condition present in the mammal that is related or unrelated to the depression or mood disorder being experienced by the mammal. Examples of such pharmaceutical agents include, for example, anti-angiogenic agents, anti-neoplastic agents, anti-diabetic agents, anti-infective agents, pain-relieving agents, anti-psychotic agents, gastrointestinal agents, etc., or combinations thereof. Other pharmaceutical agents useful in the practice of the present invention include, for example, adjunctive therapies typically used to enhance the effects of an antidepressant. Such adjunctive agents may include, for instance, mood stabilizers (e.g., lithium, valproic acid, carbamazepine, etc.); pindolol, stimulants (e.g., methylphenidate, dextroamphetamine, etc.); or thyroid augmenting agents (e.g., T₃); anti-psychotics, anti-anxiety agents (e.g., benzodiazepines), and/or agents that relieve sexual dysfunction (e.g., buspirone, which also has anti-anxiety effects; dopaminergic agents such as amantadine, pramipexole, bupropion, etc.).

As 5-HT_{2C} modulators, compounds of the present invention are useful for treating a variety of disorders. Such disorders include premenstrual syndrome (PMS), premenstrual dysphoric disorder (PMDD), motion or motor disorders such as Parkinson's disease; chronic fatigue syndrome, anorexia nervosa, disorders of sleep (e.g., sleep apnea), and mutism.

Premenstrual dysphoric disorder, or PMDD, is a severe form of PMS. Like PMS, PMDD typically occurs the week before the onset of menstruation and disappears a few days after. PMDD is characterized by severe monthly mood swings and physical symptoms that interfere with everyday life, especially a woman's relationships with her family and friends. PMDD symptoms go far beyond what are considered manageable or normal premenstrual symptoms.

PMDD is a combination of symptoms that may include irritability, depressed mood, anxiety, sleep disturbance, difficulty concentrating, angry outbursts, breast tenderness and bloating. The diagnostic criteria emphasize symptoms of depressed mood, anxiety, mood swings or irritability. The condition affects up to one in 20 American women who have regular menstrual periods. According to another embodiment, the present invention provides a method for treating one or more symptoms associated with PMDD.

Selective serotonin reuptake inhibitors (SSRIs) are the current preferred method for treating symptoms associated with PMDD. According to another aspect, the present invention provides a method for treating PMDD, or one or more symptoms associated with PMDD, by administering a compound of formula I in combination with an SSRI. In certain embodiments, the SSRI is fluoxetine, venlafaxine, paroxetine, duloxetine, or sertraline.

According to another embodiment, compounds of the present invention are useful for treating a variety of eating disorders. In certain embodiments, the eating disorder is hyperphagia, bulimia or anorexia nervosa. In certain embodiments, compounds of the present invention are useful for treating gastrointestinal disorders, such as malfunction of gastrointestinal motility or intestinal propulsion. Compounds of the present invention are also useful in connection with weight loss or control (e.g., reduction in calorie or food intake, and/or appetite suppression). Such methods are particularly useful for treating obesity with its consequent comorbidities including diabetes insipidus, Type II diabetes, cardiovascular disease, hypertension, hyperlipidemia, stroke, osteoarthritis, sleep apnea, gall bladder disease, gout, some cancers, some infertility, and early mortality.

In certain embodiments, compounds of the present invention are administered in combination with one or more anti-obesity agents. Such anti-obesity agents are known in the art and include apolipoprotein-B secretion/microsomal triglyceride transfer protein (apo-B/MTP) inhibitors, 11β-hydroxy steroid dehydrogenase-1 (11(β-HSD type 1) inhibitors, PYY_{3.36} and analogs thereof, MCR-4 agonists, cholecystokinin-A (CCK-A) agonists, monoamine reuptake inhibitors (such as sibutramine), sympathomimetic agents, R3 adrenergic receptor agonists, dopamine agonists (such as bromocriptine), melanocyte-stimulating hormone receptor analogs, cannabinoid 1 receptor antagonists (e.g., rimonabant), melanin concentrating hormone antagonists, leptins (the OB protein), leptin analogs, leptin receptor agonists, galanin antagonists, lipase inhibitors (such as tetrahydrolipstatin, i.e. orlistat), anorectic agents (such as a bombesin agonist), Neuropeptide-Y receptor antagonists, thyromimetic agents, dehydroepiandrosterone or an analog thereof, glucocorticoid receptor agonists or antagonists, orexin receptor antagonists, urocortin binding protein antagonists, glucagon-like peptide-1 receptor agonists, ciliary neurotrophic factors (such as Axokine^{TA}), human agouti-related proteins (AGRP), ghrelin receptor antagonists, histamine 3 receptor antagonists or inverse agonists, and neuromedin U receptor agonists.

In other embodiments, a compound of the present invention is administered in combination with an anti-obesity agent selected from orlistat, sibutramine, bromocriptine, ephedrine, leptin, rimonabant, pseudoephedrine, PYY3.36 or an analog thereof, and 2-oxo-N-(5-phenylpyrazinyl)spiro-[isobenzofuran-1(3H),4′-piperidine]-1′-carboxamide. According to another aspect of the invention, a compound of the present invention is administered in combination with an anti-obesity agent in conjunction with typical treatments for obesity such as exercise and a sensible diet.

According to another embodiment, a compound of the present invention is administered in combination with one or more agents for treating diabetes and associated conditions. In certain embodiments, a compound of the present invention is administered in combination with one or more such agents including insulin and insulin analogs (e.g., LysPro Insulin); GLP-1 (7-37) (insulinotropin) and GLP-1 (7-36)—NH₂; sulfonylureas and analogs thereof: chlorpropamide, glibenclamide, tolbutamide, tolazamide, acetohexamide, Glypizide®, glimepiride, repaglinide, meglitinide; biguanides: metformin, phenformin, buformin; “2-antagonists and imidazolines: midaglizole, isaglidole, deriglidole, idazoxan, efaroxan, fluparoxan; other insulin secretagogues: linogliride, A-4166; glitazones: ciglitazone, Actos® (pioglitazone), englitazone, troglitazone, darglitazone, Avandia® (BRL49653); fatty acid oxidation inhibitors: clomoxir, etomoxir; glucosidase inhibitors: acarbose, miglitol, emiglitate, voglibose, MDL-25,637, camiglibose, MDL-73,945; 13-agonists: BRL 35135, BRL 37344, RO 16-8714, ICI D7114, CL 316,243; or phosphodiesterase inhibitors: L-386,398.

In other embodiments, a compound of the present invention is administered in combination with one or more lipid-lowering agents: benfluorex: vanadate and vanadium complexes (e.g., Nagiivan®) and peroxovanadium complexes; amylin antagonists; glucagon antagonists; gluconeogenesis inhibitors; somatostatin analogs; antilipolytic agents: nicotinic acid, acipimox, WAG 994, pramlintide (Symlin”), AC 2993, nateglinide, aldose reductase inhibitors (e.g., zopolrestat), glycogen phosphorylase inhibitors, sorbitol dehydrogenase inhibitors, sodium-hydrogen exchanger type 1 (NNE-1) inhibitors and/or cholesterol biosynthesis inhibitors or cholesterol absorption inhibitors, especially a HMG-CoA reductase inhibitor, or a HMG-CoA synthase inhibitor, or a HMG-CoA reductase or synthase gene expression inhibitor, a CETP inhibitor, a bile acid sequesterant, a fibrate, an ACAT inhibitor, a squalene synthetase inhibitor, or an anti-oxidant. In other embodiments, a compound of the present invention is administered in combination with one or more naturally occurring compounds that acts to lower plasma cholesterol levels. Such naturally occurring compounds are commonly referred to as nutraceuticals and include, for example, garlic extract, Hoodia plant extracts, and niacin.

In certain embodiments, compounds of the present invention are useful for inducing, assisting or maintaining desirable bladder control in a mammal. The methods are particularly useful for treating a mammal that is experiencing or susceptible to bladder instability or urinary incontinence. Inventive methods include prevention, treatment or inhibition of bladder-related urinary conditions and bladder instability, including idiopathic bladder instability, nocturnal enuresis, nocturia, voiding dysfunction and urinary incontinence (including, for example, stress incontinence, urge incontinence, and/or mixed incontinence). Also treatable or preventable by administration of a compound of this invention is bladder instability secondary to prostate hypertrophy, as is a method for enhancing urethral tone and reducing undesirable urine leakage even in an otherwise healthy person. For example, the inventive methods are applicable to alleviating urine leakage often occurring in women during the first year after childbirth.

In other embodiments, the present compounds are useful for treating urine retention or detrusor sphinctor dyssynergia. Patients suffering from urine retention include those suffering from spinal cord injuries or male patients with benign prostatic hyperplasia.

According to the present invention, a compounds of the present invention is also useful in promoting the temporary delay of urination whenever desirable. Such compounds may be utilized in accordance with the present invention to stabilize the bladder in any applicable context. Inventive methods therefore may be utilized to allow a recipient to control the urgency and frequency of urination.

In some embodiments of the invention, compounds of the present invention are administered to a mammal in need thereof for the treatment, prevention, inhibition and/or amelioration of urge urinary incontinence (also known as bladder instability, neurogenic bladder, voiding dysfunction, hyperactive bladder, detrusor overactivity, detrusor hyper-reflexia or uninhibited bladder) or mixed urinary incontinence. Inventive uses include, but are not limited to, those for bladder activities and instabilities in which the urinary urgency is associated with prostatitis, prostatic hypertrophy, interstitial cystitis, urinary tract infections or vaginitis. The methods of this invention may also be used to assist in inhibition or correction of the conditions of Frequency-Urgency Syndrome, and lazy bladder, also known as infrequent voiding syndrome.

Compounds of the present invention may also be used to treat, prevent, inhibit, or limit the urinary incontinence, urinary instability or urinary urgency associated with or resulting from administrations of other medications, including diuretics, vasopressin antagonists, anticholinergic agents, sedatives or hypnotic agents, narcotics, alpha-adrenergic agonists, alpha-adrenergic antagonists, or calcium channel blockers.

Compounds of the present invention are useful for inducing or assisting in urinary bladder control or preventing or treating the maladies described herein in humans in need of such relief, including adult and pediatric uses. They may also be utilized for veterinary applications, particularly including canine and feline bladder control methods. If desired, the methods herein may also be used with farm animals, such as ovine, bovine, porcine and equine breeds.

According to the present invention, compounds of the present invention may be administered alone to modulate bladder activity, or alternatively may be administered in combination with (whether simultaneously or sequentially) one or more other pharmaceutical agents useful in the modulation of bladder activity. Alternatively or additionally, the compounds of the present invention may be administered in combination with one or more other pharmaceutical agents useful in the treatment or prevention of one or more other symptoms, disorders, or diseases suffered by the individual in need of bladder activity modulation.

Other pharmaceutical agents useful in the modulation of bladder activity, and particularly for treatment, prevention, inhibition, and/or amelioration of urinary incontinence, include, for example, desmopressin acetate (available as DDAVP® Nasal Spray and DDAVP® tablets from Aventis Pharmaceuticals), as well as a desmopressin acetate rhinal tube (available from Ferring Pharmaceuticals Inc.). Other products include, for example, tolterodine tartrate (available as Detroltm tablets from Pharmacia & Upjohn), oxybutynin chloride (available in the form of Ditropan® tablets and syrup and Ditropan XL® extended release tablets from ALZA Pharmaceuticals), propantheline bromide (available in tablet form from Roxane Laboratories, Inc.), hyoscyamine and hyoscyamine sulfate (available, respectively, as Cystopaz® tablets and Cystopaz-M® timed release capsules from PolyMedica Pharmaceuticals (U.S.A.), Inc.), hyoscyamine hydrobromide, flavoxate HCl (available in Urispas® 100 mg tablets from ALZA Pharmaceuticals), imipramine HCl (available in 10 mg, 25 mg and 50 mg tablets from Geneva Pharmaceuticals, Inc.), phenylpropanolamine, midodrine HCl (available in 2.5 mg and 5 mg Proamatine® tablets from Shire US Inc.), phenoxybenzamine HCl (available as Dibenzyline® capsules from WellSpring Pharmaceuticals Corporation), and prazosin HCl (available in Minipress® capsules from Pfizer Inc.). Each of these medicaments may be administered in the pharmaceutically effective amounts and regimens known in the art, including those listed in the Physicians' Desk Reference, 55 Edition, 2001, published by Medical Economics Company, Inc. at Monvale, N.J. 07645-1742, the relevant portions of which are incorporated herein by reference.

Yet other pharmaceutical agents that can act to modulate bladder activity include, for example, other regulators of the 5HT_{2C} receptor. For example, United States Patent Application 2004/0235856 (previously incorporated herein by reference in its entirety) describes a variety of 5HT_{2C} receptor modulators that are useful in accordance with the practice of the present invention. Additional 5HT_{2C} agonists are exemplified in Bishop et al., Expert Opin. Ther. Patent 13:1691-1705, 2003, the entire contents of which are incorporated herein by reference.

Still other pharmaceutical agents that can act to modulate bladder activity include, for example, modulators of one or more KCNQ potassium channels. In some embodiments of the present invention, compounds of the present invention are administered in conjunction with one or more agonists of KCNQ 2/3 or KCNQ3/5. Such KCNQ modulators include, for example, compounds described in U.S. Pat. No. 5,384,330 and those described in U.S. Pat. No. 5,565,483, as well as those described in United States Patent Application Number 2002/0183395; and United States Patent Application Number 2004/0029949. The entire contents of each of these patents and patent applications is incorporated herein by reference. In some embodiments of the present invention, compounds of the present invention are administered with retigabine.

In some embodiments of the present invention, compounds of the present invention are administered in conjunction with one or more compounds which act as vasopressin agonists including, but not limited to those described in U.S. Pat. No. 6,194,407 (Failli et al.), U.S. Pat. No. 6,090,803 (Failli et al.), U.S. Pat. No. 6,096,736 (Ogawa et al.), and U.S. Pat. No. 6,096,735 (Ogawa et al.).

In general, it will often be desirable in accordance with the present invention to administer one or more compounds of the present invention in conjunction with one or more alpha-adrenergic receptor agonists and/or one or more other sympathomimetic drugs.

According to the present invention, compounds of formula I may be used to treat, prevent, or alleviate dependence, withdrawal, or symptoms thereof for any of a variety of substances including, for example, recreational substances (e.g., alcohol, tobacco [for example, nicotine]), pharmacologic agents (e.g., pain relievers [for example, Vicodin®, Lortab®, Lorcet®, Percocet®, Percodan®, Tylox®, Hydrocodone, OxyContin®, methadone, Tramadol, etc], tranquilizers, stimulants, or sedatives), and illicit drugs (e.g., marijuana, heroine, cocaine, ecstasy, LSD, PCP, methamphetamine, etc.).

The term “substance abuse”, as used herein, may be defined with reference to criteria set form in the Diagnostic and Statistical Manual of Mental Disorders, 4^{th} Ed. (1994) (“DSM-IV”), which was prepared by the Task Force on Nomenclature and Statistics of the American Psychiatric Association. A feature of substance abuse is a maladaptive pattern of substance use manifested by recurrent and significant adverse consequences related to the repeated use of substances. As recited in the DSM-IV, substance abuse is defined as maladaptive pattern of substance abuse leading to clinically significant impairment or distress, as manifested by one (or more) of the following, occurring within a 12-month period: (1) recurrent substance use resulting in a failure to fulfill major role obligations at work, school, or home; (2) recurrent substance use in situations in which it is physically hazardous; (3) recurrent substance-related legal problems; and (4) continued substance use despite having persistent or recurrent social or interpersonal problems cause or exacerbated by the effects of the substance. In addition, the DSM-IV requires that the symptoms of substance abuse do not meet the criteria for substance dependence.

The term “substance dependence”, as used herein, may be defined with reference to criteria set form in the Diagnostic and Statistical Manual of Mental Disorders, 4^{th} Ed. (1994) (“DSM-IV”), which was prepared by the Task Force on Nomenclature and Statistics of the American Psychiatric Association. The criteria for substance dependence set forth in DSM-IV is a pattern of substance use, leading to clinically significant impairment or distress as manifested by at least three selected from the following group, occurring at any time within the same twelve month period: (1) tolerance as defined by either (a) a need for substantially increased amounts of the substance to achieve the desired effect; or (b) substantially diminished effect with continued use of the same amount of the substance; (2) withdrawal, as demonstrated by either (a) the characteristic withdrawal syndrome for the specific substance; or (b) the same, or a closely related substance is taken to relieve or avoid withdrawal symptoms; (3) the substance is often taken in larger amounts or over a longer period then was intended; (4) there is a persistent desire or unsuccessful efforts to cut down or control substance use; (5) a great deal of time is spent in activities to obtain the substance, use the substance, or recover from its effects; (6) important social, occupational or recreational activities are given up or reduced because of substance use; and (7) the substance use is continued despite knowledge of having a persistent or recurrent physical or psychological problem that is likely to have been caused or exacerbated by the substance. Substance dependence can be with physiological dependence; that is evidence of tolerance or withdrawal is present, or without physiological dependence, where no evidence of tolerance or withdrawal is present. Four of the conditions set forth in DSM-IV include remission. These types of remission are based on the interval of time that has elapsed since the cessation of dependencies and whether there is continued presence of one or more of the symptoms included in the criteria for dependencies.

In certain embodiments, compounds of the present invention are useful for treating alcoholism (e.g., alcohol abuse, addiction and/or dependence including treatment for abstinence, craving reduction and relapse prevention of alcohol intake) and/or tobacco abuse (e.g., smoking addiction, cessation and/or dependence including treatment for craving reduction and relapse prevention of tobacco smoking).

In evaluating substance abuse in accordance with the present invention, reference may be made, for example, to the National Survey on Drug Use and Health (NSDUH), which obtains information on nine different categories of illicit drug use: marijuana, cocaine, heroin, hallucinogens, inhalants, and nonmedical use of prescription-type pain relievers, tranquilizers, stimulants, and sedatives. In these categories, hashish is included with marijuana, and crack is considered a form of cocaine. Several drugs are grouped under the hallucinogens category, including LSD, PCP, peyote, mescaline, mushrooms, and “Ecstasy” (MDMA). Inhalants include a variety of substances, such as amyl nitrite, cleaning fluids, gasoline, paint, and glue. The four categories of prescription-type drugs (pain relievers, tranquilizers, stimulants, and sedatives) cover numerous drugs available through prescriptions and sometimes illegally “on the street.” Methamphetamine is considered a type of stimulant. Respondents are asked to report only uses of drugs that were not prescribed for them or drugs they took only for the experience or feeling they caused. Over-the-counter drugs and legitimate uses of prescription drugs are not included. NSDUH reports combine the four prescription-type drug groups into a category referred to as “any psychotherapeutics.”

The NSDUH categorizes alcohol abuse through use of questions about the frequency of the consumption of alcoholic beverages, such as beer, wine, whiskey, brandy, and mixed drinks. An extensive list of examples of the kinds of beverages covered is given to respondents prior to the question administration. A “drink” is defined as a can or bottle of beer, a glass of wine or a wine cooler, a shot of liquor, or a mixed drink with liquor in it. Times when the respondent only had a sip or two from a drink are not considered as consumption. For this report, estimates for the prevalence of alcohol use are reported primarily at three levels defined for both males and females and for all ages as follows:

Current use—At least one drink in the past 30 days (includes binge and heavy use).
Binge use—Five or more drinks on the same occasion at least once in the past 30 days (includes heavy use).
Heavy use—Five or more drinks on the same occasion on at least 5 different days in the past 30 days

The NSDUH also characterizes the use of tobacco products, including cigarettes, chewing tobacco, snuff, cigars, and pipe tobacco. For analytic purposes, data for chewing tobacco and snuff are combined as “smokeless tobacco.” Cigarette use is defined as smoking “part or all of a cigarette.” Questions to determine nicotine dependence among current cigarette smokers also are included in NSDUH. Nicotine dependence is based on criteria from the Nicotine Dependence Syndrome Scale (NDSS) or the Fagerstrom Test of Nicotine Dependence (FTND).

In other embodiments, compounds of the present invention are useful for treating withdrawal from drug addiction including addiction to nicotine, alcohol, and other substances of abuse. Individuals often suffer the symptoms of nicotine withdrawal as a consequence of the discontinued use of tobacco in any form, including, but not limited to smoking of cigarette, cigar, or pipe tobacco, or the oral or intranasal ingestion of tobacco or chewing tobacco. Such oral or intranasal tobacco includes, but is not limited to snuff and chewing tobacco. The cessation of nicotine use or reduction in the amount of nicotine use, is often followed within 24 hours by symptoms including dysphoric, depressed mood; light-headedness; insomnia; irritability, frustration or anger; anxiety; nervous tremor; difficulty concentrating; restlessness; decreased heart rate; increased appetite or weight gain; and the craving for tobacco or nicotine. These symptoms often cause clinically significant distress or impairment in social, occupational, or other important areas of functioning.

The discontinued or reduction in administration of an opioid, typically self-administration, through injection or orally, through smoking or intranasal ingestion, often results in the presence of a characteristic opioid withdrawal condition. This withdrawal condition can also be precipitated by administration of an opioid antagonist such as naloxone or naltrexone after opioid use. Opioid withdrawal is characterized by symptoms that are generally opposite to the opioid agonist effects. These withdrawal symptoms may include anxiety; restlessness; muscle aches, often in the back and legs; craving for opioids; irritability and increased sensitivity to pain; dysphoric mood; nausea or vomiting; lacrimation; rhinorrhoea; papillary dilation; piloerection; sweating; diarrhea; yawning; fever; and insomnia. When dependence is on short-acting opioids, such as heroin, withdrawal symptoms usually occur within 6-24 hours after the last dose, while with longer-acting opioids, such as methadone, symptoms may take 2-4 days to emerge. These symptoms often cause clinically significant distress or impairment in social, occupational or other important areas of functioning. The present invention is most preferably used to alleviate one or more symptoms attributed to opioid withdrawal when such symptoms are not due to a general medical condition and are not better accounted for by another medical disorder.

The discontinued or reduction in use of ethanol (ethanol containing beverages) results in the onset of ethanol withdrawal conditions. Ethanol withdrawal conditions are characterized by symptoms that begin when blood concentrations of ethanol decline sharply, within 4 to 12 hours after ethanol use has been stopped or reduced. These ethanol withdrawal symptoms include craving for ethanol; autonomic hyperactivity (such as sweating or pulse rate greater than 100); hand tremor; insomnia; nausea; vomiting; transient visual, tactile, or auditory hallucinations or illusions; psychomotor agitation; anxiety; and grand mal seizures. These symptoms often cause clinically significant distress or impairment in social, occupational, or other important areas of functioning. The present invention is most preferably used to alleviate one or more symptoms attributed to ethanol withdrawal when such symptoms are not due to a general medical condition and are not better accounted for by another medical disorder.

According to another embodiment, a compound of the present invention is administered in combination with one or more agents useful for treating substance abuse. In certain embodiments, a compound of the present invention is administered in combination with one or more agents to treat tobacco abuse. Such agents include nicotine receptor partial agonists bupropion hydochloride (Zyban™) and nicotine replacement therapies.

According to yet another embodiment, a compound of the present invention is administered in combination with one or more agents to treat alcoholism, such as opioid antagonists (e.g., naltrexone, ReVia™), nalmefene, disulfuram (Antabuse™), and acamprosate (Campral™).

In certain embodiments, a compound is administered in combination with one or more agents for reducing alcohol withdrawal symptoms such as benzodiazepines, beta-blockers, clonidine, carbamazepine, pregabalin, and gabapentin (Neurontin™). In other embodiments of the invention, therapy utilizing compounds of the present invention is administered concomitantly with, in connection with, and/or subsequent to an educational and/or behavioral modification program to enhance continued abstinence from substance dependence or abuse. The method of the present invention may be particularly useful in treating symptoms of withdrawal often observed in rehabilitation or other treatment programs. Therefore, the programs can be more effective by focusing on educational and behavioral modification goals, further reducing the incidence of program non-completion.

In certain embodiments, compounds of the present invention are useful for treating one or more intellectual deficit disorders comprising administering a compound of the present invention. In other embodiments, such intellectual deficit disorders include dementia, such as dementia of aging, vascular dementia, mild cognitive impairment, age-related cognitive decline, and mild neurocognitive disorder; Alzheimer's disease, and memory deficit, attention deficit disorders (ADD, also known as Attention Deficit Hyperactivity Disorder or ADHD) in both children and adults. In certain embodiments, the present invention provides a method of treating ADD and/or ADHD in a pediatric patient comprising administering to said patient a compound of formula I or pharmaceutical composition thereof.

In other embodiments, the present invention provides a method of treating one or more cognition disorders. According to another aspect, the cognition disorder is a learning disorder. Such learning disorders are known in the art and include autism, dyslexia, Asperger's syndrome, a neurobiological disorder similar to autism and characterized by serious deficits in social and communication skills; specific learning disability, a disorder in one or more of the basic psychological processes involved in understanding or in using spoken or written language, which may manifest itself in an imperfect ability to listen, think, speak, read, write, spell or to do mathematical calculations; dysgraphia, a disorder that causes difficulty with forming letters or writing within a defined space; dyscalculia, a disorder that causes people to have problems doing arithmetic and grasping mathematical concepts; dyspraxia, a problem with the body's system of motion that interferes with a person's ability to make a controlled or coordinated physical response in a given situation; visual perceptual deficit, difficulty receiving and/or processing accurate information from the sense of sight, although there is nothing wrong with vision; and auditory perceptual deficit, difficulty receiving accurate information through auditory means, even though there is no problem with hearing.

In certain embodiments, the present invention provides a method for treating one or more impulsivity disorders (e.g. borderline personality disorder), disruptive behavior disorders, or impulse control disorders. In certain embodiments, the present invention provides a method for treating Tourette's syndrome (TS), an inherited, neurological disorder characterized by repeated and involuntary body movements (tics) and/or uncontrollable vocal sounds.

According to another aspect, the present invention provides a method for treating one or more behavioral addictions and addictive disorders. Behavioral addictions and addictive disorders result from the intoxication one senses from the release of brain chemicals (e.g., serotonin, adrenaline, epinephrine, etc.) during certain activities. Such disorders are known in the art and include gambling, sex addiction, eating disorders, spending addiction, rage/anger, workaholism, exercise addiction, risk taking addictions, and perfectionism to name a few.

In certain embodiments, a compound of the present invention is administered in combination with one or more cognitive improvement agents. Such agents are well known in the art and include donepezil hydrochloride (Aircept™) and other acetylcholinesterase inhibitors; galantamine, neuroprotective agents (e.g., memantine); ADD/ADHD agents (e.g., methylphenidate (Ritalin™), atomoxetine (Strattera™), methylphenidate, sustained release (Concerta™) and amphetamine/dextroamphetamine (Adderall™).

According to another aspect, the present invention provides a method for treating sexual dysfunction comprising administering a compound of the present invention. In certain embodiments, the sexual dysfunction is associated with a depressive disorder. In other embodiments, the sexual dysfunction is associated with treatment of a disorder by administration of a serotonin reuptake inhibitor. Compounds of the present invention are useful for treating sexual dysfunction in the male and in the female. Such disorders include male erectile dysfunction (MED) and female sexual dysfunction (FSD), e.g. female sexual arousal disorder (FSAD).

In other embodiments, the present invention provides a method for treating one or more disorders associated with sexual dysfunction including: HSDD, characterized by a deficiency, or absence of, sexual fantasies and desire for sexual activity; FSAD, characterized by a persistent or recurrent inability to attain, or to maintain until completion of the sexual activity, an adequate lubrication-swelling response of sexual excitement; FOD characterized by persistent or recurrent delay in, or absence of, orgasm following a normal sexual excitement phase; Sexual Pain Disorders such as dyspareunia and vaginismus; and/or HSDD characterized by a woman who has no or little desire to be sexual, and has no or few sexual thoughts or fantasies.

According to another embodiment, a compound of the present invention is administered in combination with one or more agents for treating male sexual dysfunction (e.g., male erectile dysfunction). Such agents are known in the art and include a dopaminergic agent (e.g. D2, D3 or D4 agonists and apomorphine); an NPY (neuropeptide Y) (preferably an NPY-1 and/or NPY-5 inhibitor); a melanocortin receptor agonist or modulator or melanocortin enhancer; an NEP inhibitor; a PDE inhibitor (preferably, a cGMP PDE-5 inhibitor); a bombesin receptor antagonist or modulator, and a soluble secreted endopeptidase inhibitor (SEPi). In certain embodiments, a compound of the present invention is administered in combination with one or more agents for treating male sexual dysfunction such as alprostadil or sildenafil.

According to yet another embodiment, a compound of the present invention is administered in combination with one or more agents for treating female sexual dysfunction. Such agents are known in the art and include estrogen receptor modulators (e.g., estrogen agonists and/or estrogen antagonists); testosterone replacement agents, testosterone (Tostrelle), dihydrotestosterone, dehydroepiandrosterone (DHEA), a testosterone implant; e.g. dehydroandrostendione, estrogen, estrogen, medroxyprogesterone, medroxyprogesterone acetate (MPA), a combination of estrogen and a methyl testosterone hormone replacement therapy agent; Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone, a dopaminergic agent; e.g. apomorphine or a selective D2, D3 or D2/D₃agonist such as, pramipexole and ropirinol, a NPY (neuropeptide Y) inhibito; e.g. a NPY (neuropeptide Y) inhibitor such as a NPY1 or NPY5 inhibitor, preferably NPY1 inhibitor, a melanocortin receptor modulator or a melanocortin enhancer; e.g. melanotan II, PT-14, PT-141, a NEP (neutral endopeptidase) inhibitor; a PDE (phosphodiesterase) inhibitor; e.g. sildenafil, and/or a bombesin receptor modulator.

According to the present invention, compounds of the present invention are useful for treating any of a variety of different types of pain experienced by mammals, such as humans. For example, the compounds of the present invention may be used to treat acute pain (short duration) or chronic pain (regularly reoccurring or persistent), whether centralized or peripheral.

Examples of pain that can be acute or chronic and that can be treated in accordance with the methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain, or headaches such as migraines or tension headaches, or combinations of these pains. One skilled in the art will recognize that these pains may overlap one another. For example, a pain caused by inflammation may also be visceral or musculoskeletal in nature.

In one embodiment of the present invention, one or more compounds of the present invention is/are administered in mammals to treat chronic pain such as neuropathic pain associated for example with damage to or pathological changes in the peripheral or central nervous systems; cancer pain; visceral pain associated with for example the abdominal, pelvic, and/or perineal regions or pancreatitis; musculoskeletal pain associated with for example the lower or upper back, spine, fibromyalgia, temporomandibular joint, or myofascial pain syndrome; bony pain associated with for example bone or joint degenerating disorders such as osteoarthritis, rheumatoid arthritis, or spinal stenosis; headaches such migraine or tension headaches; or pain associated with infections such as HIV, sickle cell anemia, autoimmune disorders, multiple sclerosis, or inflammation such as osteoarthritis or rheumatoid arthritis.

In some embodiments, the compounds of the present invention are used to treat chronic pain that is neuropathic pain, visceral pain, musculoskeletal pain, bony pain, headache, cancer pain or inflammatory pain or combinations thereof, in accordance with the methods described herein. Inflammatory pain can be associated with a variety of medical conditions such as osteoarthritis, rheumatoid arthritis, surgery, or injury. Neuropathic pain may be associated with for example diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury resulting in peripheral and/or central sensitization such as phantom limb pain, reflex sympathetic dystrophy or postthoracotomy pain, cancer, chemical injury, toxins, nutritional deficiencies, or viral or bacterial infections such as shingles or HIV, or combinations thereof. Inventive treatment methods further include treatments in which the neuropathic pain is a condition secondary to metastatic infiltration, adiposis dolorosa, burns or central pain conditions related to thalamic conditions.

Neuropathic pains described above may also be, in some circumstances, classified as “painful small fiber neuropathies” such as idiopathic small-fiber painful sensory neuropathy, or “painful large fiber neuropathies” such as demyelinating neuropathy or axonal neuropathy, or combinations thereof. Such neuropathies are described in more detail, for example, in the J. Mendell et al., N. Engl. J. Med. 2003, 348:1243-1255, which is hereby incorporated by reference in its entirety.

In another embodiment, the compounds useful in the present invention may be administered to totally or partially inhibit a neuropathic pain condition from developing. For example, compounds of the present invention may be administered to a mammal who is at risk for developing a neuropathic pain condition such as a mammal who has contracted shingles or a mammal who is being treated for cancer.

In one embodiment, the compounds useful in the present invention may be administered prior to or during a surgical procedure to partially or totally inhibit development of pain associated with the surgical procedure.

As mentioned previously, the methods of the present invention may be used to treat pain that is somatic and/or visceral in nature. For example, somatic pain that can be treated in accordance with the methods of the present invention includes pain associated with structural or soft tissue injury experienced during surgery, dental procedures, burns, or traumatic body injuries. Examples of visceral pain that can be treated in accordance with the methods of the present invention include those types of pain associated with or resulting from maladies of the internal organs such as ulcerative colitis, irritable bowel syndrome, irritable bladder, Crohn's disease, rheumatologic (arthralgias), tumors, gastritis, pancreatitis, infections of the organs, or biliary tract disorders, or combinations thereof. One skilled in the art will also recognize that the pain treated according to the methods of the present invention may also be related to conditions of hyperalgesia, allodynia, or both. Additionally, chronic pain to be treated in accordance with the present invention may be with or without peripheral or central sensitization.

The present invention also provides use of the compounds of the present invention to treat acute and/or chronic pains associated with female conditions, which may also be referred to as female-specific pain. Such types of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes.

In certain embodiments, a compound of the present invention is administered in combination with a pain relieving agent. Examples of pain relieving agents that may be administered with compounds of the present invention include, but are not limited to, analgesics such as non-narcotic analgesics or narcotic analgesics; anti-inflammatory agents such as non-steroidal anti-inflammatory agents (NSAIDs), steroids or anti-rheumatic agents; migraine preparations such as beta adrenergic blocking agents, ergot derivatives, or isometheptene; tricyclic antidepressants such as amitryptyline, desipramine, or imipramine; anti-epileptics such as gabapentin, carbamazepine, topiramate, sodium valproate or phenyloin; α₂ agonists; or selective serotonin reuptake inhibitors/selective norepinephrine uptake inhibitors, or combinations thereof.

One skilled in the art will recognize that some agents described herein act to relieve multiple conditions such as pain and inflammation, while other agents may just relieve one symptom such as pain. A specific example of an agent having multiple properties is aspirin, where aspirin is anti-inflammatory when given in high doses, but at lower doses is just an analgesic. The pain relieving agent may include any combination of the aforementioned agents, for example, the pain relieving agent may be a non-narcotic analgesic in combination with a narcotic analgesic.

Non-narcotic analgesics useful in the practice of the present invention include, for example, salicylates such as aspirin, ibuprofen (Motrin®, Advil®), ketoprofen (Orudis®), naproxen (Naprosyn®), acetaminophen, indomethacin or combinations thereof. Examples of narcotic analgesic agents that may be used in combination with compounds of the present invention include opioid analgesics such as fentanyl, sufentanil, morphine, hydromorphone, codeine, oxycodone, buprenorphine or pharmaceutically acceptable salts thereof or combinations thereof. Examples of anti-inflammatory agents that may be used in combination with compounds of the present invention include but are not limited to aspirin; ibuprofen; ketoprofen; naproxen; etodolac (Lodine®); COX-2 inhibitors such as celecoxib (Celebrex®), rofecoxib (Vioxx®), valdecoxib (Bextra®), parecoxib, etoricoxib (MK663), deracoxib, 2-(4-ethoxy-phenyl)-3-(4-methanesulfonyl-phenyl)-pyrazolo[1,5-b]pyridazine, 4-(2-oxo-3-phenyl-2,3-dihydrooxazol-4-yl)benzenesulfonamide, darbufelone, flosulide, 4-(4-cyclohexyl-2-methyl-5-oxazolyl)-2-fluorobenzenesulfonamide), meloxicam, nimesulide, 1-Methylsulfonyl-4-(1,1-dimethyl-4-(4-fluorophenyl)cyclopenta-2,4-dien-3-yl)benzene, 4-(1,5-Dihydro-6-fluoro-7-methoxy-3-(trifluoromethyl)-(2)-benzothiopyrano(4,3-c)pyrazol-1-yl)benzenesulfonamide, 4,4-dimethyl-2-phenyl-3-(4-methylsulfonyl)phenyl)cyclo-butenone, 4-Amino-N-(4-(2-fluoro-5-trifluoromethyl)-thiazol-2-yl)-benzene sulfonamide, 1-(7-tert-butyl-2,3-dihydro-3,3-dimethyl-5-benzo-furanyl)-4-cyclopropyl butan-1-one, or their physiologically acceptable salts, esters or solvates; sulindac (Clinoril®); diclofenac (Voltaren®); piroxicam (Feldene®); diflunisal (Dolobid®), nabumetone (Relefen®), oxaprozin (Daypro®), indomethacin (Indocin®); or steroids such as Pediaped® prednisolone sodium phosphate oral solution, Solu-Medrol® methylprednisolone sodium succinate for injection, Prelone® brand prednisolone syrup.

Further examples of anti-inflammatory agents that may be used for treating pain, for example associated with rheumatoid arthritis, in accordance with the present invention include naproxen, which is commercially available in the form of EC-Naprosyn® delayed release tablets, Naprosyn®, Anaprox® and Anaprox® DS tablets and Naprosyn® suspension from Roche Labs, Celebrex® brand of celecoxib tablets, Vioxx® brand of rofecoxib, Celestone® brand of betamethasone, Cupramine® brand penicillamine capsules, Depen® brand titratable penicillamine tablets, Depo-Medrol® brand of methylprednisolone acetate injectable suspension, Arava™ leflunomide tablets, Azulfidine EN-tabs® brand of sulfasalazine delayed release tablets, Feldene® brand piroxicam capsules, Cataflam® diclofenac potassium tablets, Voltaren® diclofenac sodium delayed release tablets, Voltaren®-XR diclofenac sodium extended release tablets, or Enbrel® etanerecept products.

Examples of yet other agents used to treat inflammations, especially rheumatoid arthritis, include immunosuppressants such as Gengraf™ brand cyclosporine capsules, Neoral® brand cyclosporine capsules or oral solution, or Imuran® brand azathioprine tablets or IV injection; Indocin® brand indomethacin capsules, oral suspension or suppositories; Plaquenil® brand hydroxychloroquine sulfate; or Remicade® infliximab recombinant for IV injection; or gold compounds such as auranofin or Myochrisyine® gold sodium thiomalate injection.

In other embodiments, compounds of the present invention are useful for treating one or more central nervous system deficiencies associated, for example, with trauma, stroke, and spinal cord injuries, neurodegenerative diseases or toxic or infective CNS diseases (e.g., encephalitis or meningitis), or Parkinson's disease. The compounds of the present invention can therefore be used to improve or inhibit further degradation of central nervous system activity during or following the malady or trauma in question. Included in these improvements are maintenance or improvement in motor and motility skills, control, coordination and strength.

### 5. Pharmaceutically Acceptable Compositions

In other embodiments, the invention relates to compositions comprising at least one compound of formula I, or a pharmaceutically acceptable salt thereof, and one or more pharmaceutically acceptable carriers, excipients, or diluents. Such compositions include pharmaceutical compositions for treating or controlling disease states or conditions of the central nervous system. In certain embodiments, the compositions comprise mixtures of one or more compounds of formula I.

In certain embodiments, the invention relates to compositions comprising at least one compound of formula I, or a pharmaceutically acceptable salt thereof, and one or more pharmaceutically acceptable carriers, excipients, or diluents. Such compositions are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those carriers that are compatible with the other ingredients in the formulation and are biologically acceptable.

The compounds of formula I can be administered orally or parenterally, neat, or in combination with conventional pharmaceutical carriers. Applicable solid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers can be used in preparing solutions, suspensions, emulsions, syrups and elixirs. The active ingredient can be dissolved or suspended in a pharmaceutically acceptable liquid carrier such as water, an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

The compounds of formula I can be administered rectally or vaginally in the form of a conventional suppository. For administration by intranasal or intrabronchial inhalation or insufflation, the compounds of formula I can be formulated into an aqueous or partially aqueous solution, which can then be utilized in the form of an aerosol. The compounds of Formula I can also be administered transdermally through the use of a transdermal patch containing the active compound and a carrier that is inert to the active compound, is non-toxic to the skin, and allows delivery of the agent for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable. A variety of occlusive devices can be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

The amount of compound of formula I provided to a patient will vary depending upon what is being administered, the purpose of the administration, such as prophylaxis or therapy, the state of the patient, the manner of administration, and the like. In therapeutic applications, compounds of formula I are provided to a patient suffering from a condition in an amount sufficient to treat or at least partially treat the symptoms of the condition and its complications. An amount adequate to accomplish this is a “therapeutically effective amount” as described previously herein. The dosage to be used in the treatment of a specific case must be subjectively determined by the attending physician. The variables involved include the specific condition and the size, age, and response pattern of the patient. The treatment of substance abuse follows the same method of subjective drug administration under the guidance of the attending physician. Generally, a starting dose is about 5 mg per day with gradual increase in the daily dose to about 1000 mg per day, to provide the desired dosage level in the patient.

### 6. Combination with Other Agents

Compounds of formula I may be administered alone in order to treat various disorders in accordance with the present invention, or may be combined with one or more other pharmaceutical agents as described herein. Where the present invention involves administration of two or more pharmaceutical agents, the two or more agents may be administered simultaneously (such as individually at the same time, or together in a pharmaceutical composition), and/or successively with one another. In general, a compound of formula I and the other pharmaceutical agent(s) are administered in a manner so that both are present in the mammal body for a certain period of time to treat the disorder.

Also, the two or more pharmaceutical agents may be delivered via the same route of administration or by different routes. Desirable routes of administration may well depend upon the particular agent(s) chosen, many of which have recommended administration route(s) known to those skilled in the art. For example, opioids are generally administered by oral, intravenous, or intramuscular administration routes. Similarly, as is known in the art, doses of pharmaceutical agents in a composition may be affected by administration route. In general, pharmaceutical agents may be dosed and administered according to practices known to those skilled in the art such as those disclosed in references such as the Physicians' Desk Reference, 55 Edition, 2001, published by Medical Economics Co., Inc., Montvale, N.J.

A more complete list of pharmaceutically active agents, including pain relieving agents, can be found in the Physicians' Desk Reference, 55 Edition, 2001, published by Medical Economics Co., Inc., Montvale, N.J. Each of these agents may be administered in conjunction with one or more compounds of formula I according to the present invention. For most or all of these agents, recommended effective dosages and regimes are known in the art; many can be found in the above-referenced Physicians' Desk Reference, 55 Edition, 2001, published by Medical Economics Co., Inc., Montvale, N.J.

In certain embodiments, the present invention is directed to prodrugs of compounds of formula I. The term “prodrug,” as used herein, means a compound that is convertible in vivo by metabolic means (e.g. by hydrolysis) to a compound of formula I. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is hereby incorporated by reference in its entirety.

### EXAMPLES

As depicted in the Examples below, in certain exemplary embodiments, compounds are prepared according to the following general procedures. It will be appreciated that although the general methods depict the synthesis of certain compounds of the present invention, the following general methods, in addition to the Schemes set forth above and other methods known to one of ordinary skill in the art, can be applied to all compounds and subclasses and species of each of these compounds, as described herein.

The following examples illustrate the production of representative compounds of this invention.

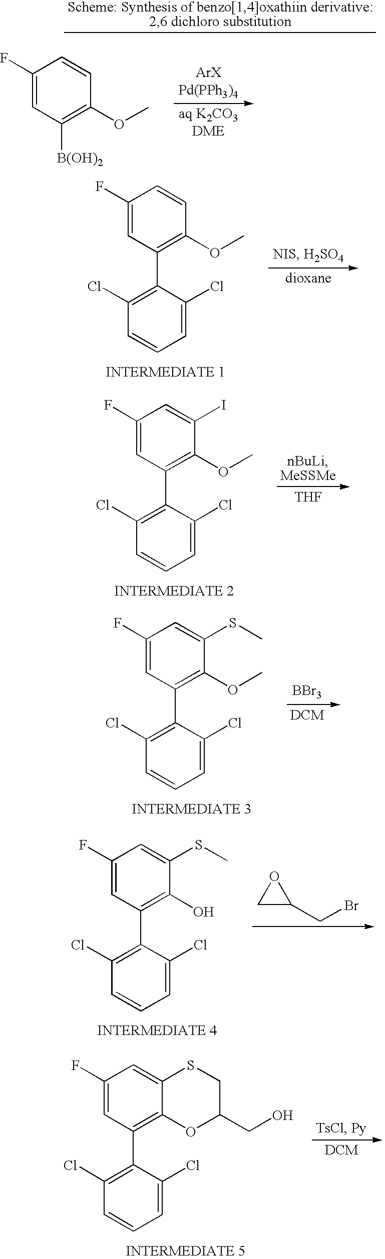

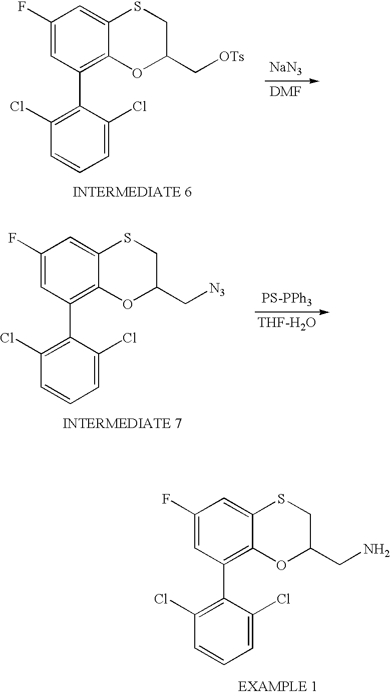

### Intermediate 1

2′,6′-Dichloro-5-fluoro-2-methoxy-biphenyl: To a solution of 2-methoxy-5-fluorophenylboronic acid (15.1 g, 88.3 mmol) and 1-bromo-2,6-dichloro benzene (11.9 g, 52.9 mmol) in degassed DME (350 mL), were added tetrakis-(triphenylphospin)-palladium(0) (3.06 g, 2.65 mmol) and 2M aqueous potassium carbonate (66.2 mL, 132.5 mmol). The reaction mixture was allowed to stir at 90° C. for 48 h, then cooled to room temperature and concentrated under vacuum. The concentrated crude was diluted with diethyl ether (150 mL), and filtered through celite. The organic layer was washed with water (2×150 mL) and saturated aqueous sodium chloride (100 mL), dried (sodium sulfate), and the solvent was removed in vacuo to provide a crude oil. Purification by flash column chromatography (Silica, Hexane:AcOEt 95:5) afforded 11.40 g of the title compound (80% yield). MS (ESI) m/z detected mass 285.9/288.3 [M+H]⁺; expected mass 271/273.

¹H NMR (300 MHz, CHLOROFORM-d) d ppm 7.39-7.44 (m, 2H) 7.26 (dd, 1H) 7.12 (ddd, 1H) 6.96 (dd, 1H) 6.90 (dd, 1H) 3.77 (s, 3H).

### Intermediate 2

2′,6′-Dichloro-5-fluoro-3-iodo-2-methoxy-biphenyl: To a magnetically stirred solution of 2′,6′-Dichloro-5-fluoro-2-methoxy-biphenyl (20 g, 0.073 mol), in 100 mL of dioxane, conc. Sulphuric acid (19.5 mL) was added. NIS (19.9 g, 0.088 mol) was added in one portion to the warm solution. The mixture was heated at 50° C. for 18 h. The reaction mixture was cooled at room temperature and poured into ice. DCM was added and the mixture was transferred into a separator funnel. The aqueous layer was separated and extracted with additional portion of DCM. The combined organics were washed once with water, then aq. Na₂S₂O₃ solution and finally with 1 M aq. NaOH. The organic layer was dried (sodium sulfate), filtered and evaporated under vacuum. Purification by flash column chromatography (Silica, Hexanes:Acetone 100:1 to 100:5) afforded 19.6 g of the title compound (68% yield). MS (ESI) m/z 395.8/397.8 [M+H]⁺.

¹H NMR (300 MHz, DMSO-d6, 373 K) d ppm 7.75 (dd, 1H) 7.54-7.59 (m, 2H) 7.47 (dd, 1H) 7.09 (dd, 1H) 3.45 (s, 3H).

### Intermediate 3

2′,6′-Dichloro-5-fluoro-2-methoxy-3-methylsulfanyl-biphenyl: A nitrogen-flushed solution of 2′,6′-Dichloro-5-fluoro-3-iodo-2-methoxy-biphenyl (5.0 g, 12.6 mmol) in dry THF (125 mL) was cooled at −78° C. A 2.5 M solution of n-BuLi in hexane (6.05 mL, 15.1 mmol) was added dropwise. The reaction mixture was keeped at −78° C. and methyldisulfanylmethane (2.24 mL, 25.2 mmol) was added. The mixture was stirred at −78° C. for 4 hours. The reaction was quenched with water and the solvent was evaporated under vacuum. The crude was dissolved in DCM and washed twice with water. The organic layer was dried over anhydrous sodium sulfate, filtered and the solvent was evaporated under vacuum. Purification by flash column chromatography (Silica, Hexanes to Hexanes:DCM 85:15) afforded 2.3 g of the title compound (57% yield). MS (ESI) m/z 316.9/318.9 [M+H]⁺.

¹H NMR (300 MHz, DMSO-d6, 373 K) d ppm 7.52-7.59 (m, 2H) 7.46 (dd, 1H) 7.34 (dd, 1H) 6.82 (dd, 1H) 4.27 (q, 2H) 4.27 (q, 2H) 1.22 (t, 6H).

### Intermediate 4

2′,6′-Dichloro-5-fluoro-3-methylsulfanyl-biphenyl-2-ol: To a solution of 2′,6′-Dichloro-5-fluoro-2-methoxy-3-methylsulfanyl-biphenyl (1.32 mmol, 418 mg) in dry DCM (20 mL), was added a 1 M solution of boron tribromide in DCM (1.38 mmol, 1.38 mL). The reaction mixture was stirred at 0° C. for 4 h then warmed to room temperature and stirred for additional 12 h. A saturated solution of NaHCO₃ was added and the organic layer separated, dried (sodium sulfate) and evaporated under vacuum. Purification by flash column chromatography, (Silica, Hexanes:AcOEt 95:5) afforded 143 mg of pure title compound (36% yield). MS (ESI) m/z 302.0/304.0 [M+H]⁺.

¹H NMR (300 MHz, DMSO-d6) d ppm 8.78 (br. s., 1H) 7.51-7.58 (m, 2H) 7.42 (dd, 1H) 7.04 (dd, 1H) 6.70 (dd, 1H) 2.43 (s, 3H).

### Intermediate 5

[8-(2,6-Dichloro-phenyl)-6-fluoro-2,3-dihydro-benzo[1,4]oxathiin-2-yl]-methanol:
2′,6′-Dichloro-5-fluoro-3-methylsulfanyl-biphenyl-2-ol (1.03 mmol, 0.313 g) was dissolved in epibromohydrine (2.3 mL). 4 Å molecular sieves were added and the reaction mixture was heated at 150° C. under microwave irradiation for 70 minutes. Molecular sieves were filtered off and a 1 N solution of NaOH (3 mL) was added to the reaction. The mixture was extracted with ethyl ether (3×5 mL). The combined organics were dried (sodium sulfate) and evaporated under vacuum. Purification by flash chromatography, (Silica, Hexanes: AcOEt 7:3) afforded 100 mg of pure title compound (28% yield). MS (ESI) m/z 344.9/346.7 [M+H]⁺.

¹H NMR (300 MHz, CHLOROFORM-d) d ppm 7.39-7.46 (m, 2H) 7.27 (dd, 2H) 6.92 (dd, 1H) 6.66 (dd, 1H) 4.22-4.30 (m, 1H) 3.67-3.73 (m, 1H) 3.16 (dd, 1H) 2.99 (dd, 1H) 1.72 (dd, 1H).

### Intermediate 6

Toluene-4-sulfonic acid 8-(2,6-dichloro-phenyl)-6-fluoro-2,3-dihydro-benzo[1,4]oxathiin-2-ylmethyl ester: A solution of [8-(2,6-Dichloro-phenyl)-6-fluoro-2,3-dihydro-benzo[1,4]oxathiin-2-yl]-methanol (0.16 g, 0.46 mmol) and pyridine (0.11 mL, 1.39 mmol) in CH₂Cl₂ (10 mL) was cooled at 0° C. and p-toluenesulfonyl chloride (0.26 g, 1.39 mmol) was added. The reaction mixture was stirred at RT for 12 h and then diluted with methylene chloride (100 mL), washed with 1N HCl solution and brine. The organic layer was dried (sodium sulfate) and evaporated under vacuum. Purification by flash chromatography, (Silica, Hexanes:DCM 1:1) afforded 0.14 g (63%) of the title compound. MS (ESI) m/z 498.9/501.3 [M+H]⁺.

¹H NMR (300 MHz, CHLOROFORM-d) d ppm 7.67-7.73 (m, 2H) 7.30-7.40 (m, 4H) 7.24 (dd, 1H) 6.87 (dd, 1H) 6.63 (dd, 1H) 4.35-4.44 (m, 1H) 4.06-4.11 (m, 2H) 2.96-3.09 (m, 2H) 2.46 (s, 3H).

### Intermediate 7

2-Azidomethyl-8-(2,6-dichloro-phenyl)-6-fluoro-2,3-dihydro-benzo[1,4]oxathiine: To a solution of toluene-4-sulfonic acid 8-(2,6-dichloro-phenyl)-6-fluoro-2,3-dihydro-benzo[1,4]oxathiin-2-ylmethyl ester (0.14 g, 0.278 mmol) in DMF (15 mL), NaN₃ (0.09 g, 1.39 mmol) was added under inert atmosphere and the reaction mixture was allowed to stir at 90° C. for 16 h. The solvent was concentrated under vacuum and the residue portioned between brine and Et₂O. The organic layer was dried (sodium sulfate) and concentrated under vacuum. The product obtained was used as such in the next step.

MS (ESI) m/z 341.9/343.9 [M+H] as major fragment (loss of N₂).

### Example 1

C-[8-(2,6-Dichloro-phenyl)-6-fluoro-2,3-dihydro-benzo[1,4]oxathiin-2-yl]-methylamine (hydrochloric salt): To a suspension of swelled PS-PPh₃ (0.232 g, 0.56 mmol, loading 2.4 mmol/g) in THF (10 mL), a solution of 2-Azidomethyl-8-(2,6-dichloro-phenyl)-6-fluoro-2,3-dihydro-benzo[1,4]oxathiine (theoretical 0.278 mmol) in THF (2 mL) was added. The reaction was allowed to shake at room temperature for 16 h. H₂O (2 mL) was added to the mixture and the reaction was shaked for additional 16 h. The resin was filtered off and washed with DCM. The solution was loaded on a strong cation exchange resin and the product was eluted with a 3% solution of NH₄OH in MeOH. The solvent was removed under vacuum and the residue dissolved in DCM. A solution of gaseous HCl in Et₂O was added and the solution was stirred at room temperature for 10 minutes. The solvent was removed under vacuum to afford the title compound as hydrochloric salt (91.2 mg, 86% yield over two steps).

MS (ESI) m/z 343.9/345.9 [M+H]⁺.

¹H NMR (300 MHz, CHLOROFORM-d) d ppm 7.67-7.73 (m, 2H) 7.30-7.40 (m, 4H) 7.24 (dd, 1H) 6.87 (dd, 1H) 6.63 (dd, 1H) 4.35-4.44 (m, 1H) 4.06-4.11 (m, 2H) 2.96-3.09 (m, 2H) 2.46 (s, 3H).

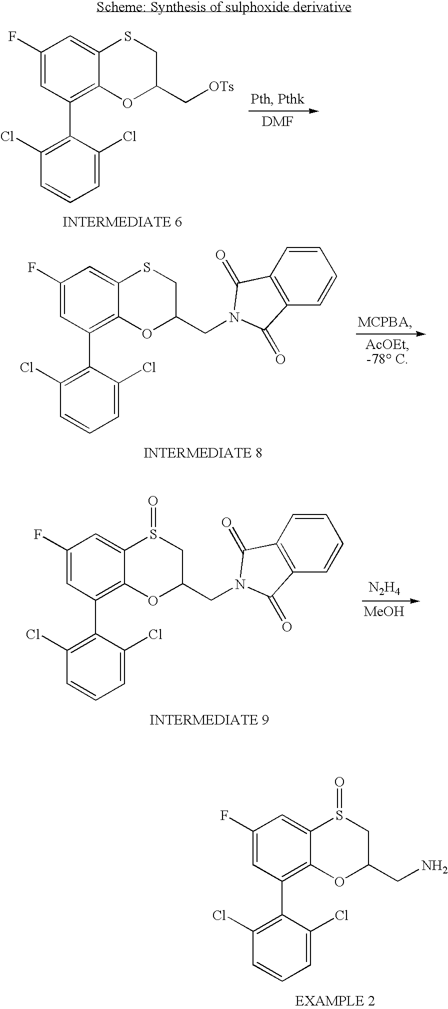

INTERMEDIATE 6 was obtained as previously described.

### Intermediate 8

2-[8-(2,6-Dichloro-phenyl)-6-fluoro-2,3-dihydro-benzo[1,4]oxathiin-2-ylmethyl]-isoindole-1,3-dione: Under an inert atmosphere Toluene-4-sulfonic acid 8-(2,6-dichloro-phenyl)-6-fluoro-2,3-dihydro-benzo[1,4]oxathiin-2-ylmethyl ester (0.177 g, 0.35 mmol) was dissolved in anhydrous DMF (10 mL). Phthalimide (0.055 g, 0.37 mmol) and Phthalimide K-salt (0.069 g, 0.37 mmol) were added and the reaction mixture was stirred at 75° C. for 15 h. The reaction mixture was concentrated under vacuum, diluted with AcOEt and washed with 1N NaOH and brine. The organic layer was separated, dried (sodium sulfate), filtered and the solvent was removed under vacuum. Purification by flash column chromatography (Silica, Hexanes:DCM 9:1) afforded 0.133 g of pure title compound (80% yield). MS (ESI) m/z 473.8/475.8 [M+H]⁺.

¹H NMR (300 MHz, CHLOROFORM-d) d ppm 7.76-7.86 (m, 4H) 7.35 (dd, 1H) 6.97 (t, 1H) 6.90 (ddd, 1H) 6.52 (dd, 1H) 6.44 (dd, 1H) 4.49-4.55 (m, 1H) 3.95 (dd, 1H) 3.75 (dd, 1H) 3.19 (dd, 1H) 3.04 (dd, 1H).

Note: When phthalimido moiety is present in the molecule, a loss of symmetry of the protons of the chlorinated ring was observed.

### Intermediate 9

2-[8-(2,6-Dichloro-phenyl)-6-fluoro-4-oxo-3,4-dihydro-2H-4lambda*4*-benzo[1,4]oxathiin-2-ylmethyl]-isoindole-1,3-dione: To a solution of 2-[8-(2,6-Dichloro-phenyl)-6-fluoro-2,3-dihydro-benzo[1,4]oxathiin-2-ylmethyl]-isoindole-1,3-dione (0.133 g, 0.28 mmol) in AcOEt (9 mL) at −78° C. under an inert atmosphere was added MCPBA 77% (0.625 g, 0.28 mmol). The resulting clear solution was stirred at this temperature for 2.5 h and then the solvent was evaporated under vacuum. Purification by flash chromatography (Silica, DCM:MeOH 95:5) afforded 85.2 mg of pure title compound (62% yield). MS (ESI) m/z 489.7/491.7.0 [M+H]⁺.

¹H NMR (300 MHz, DMSO-d6) d ppm 7.78-7.96 (m, 5H) 7.45-7.55 (m, 1H) 7.38 (dd, 1H) 7.20 (t, 1H) 6.38 (dd, 1H) 4.73-4.87 (m, 1H) 4.02 (dd, 1H) 3.84 (dd, 1H) 3.56 (d, 1H) 3.25 (d, 1H)

Note: Similarly to Intermediate 8, the phthalimido moiety present in the molecule causes a loss of symmetry of the protons of the chlorinated ring.

### Example 2

C-[8-(2,6-Dichloro-phenyl)-6-fluoro-4-oxo-3,4-dihydro-2H-4lambda*4*-benzo[1,4]oxathiin-2-yl]-methylamine (hydrochloric salt): 2-[8-(2,6-Dichloro-phenyl)-6-fluoro-4-oxo-3,4-dihydro-2H-4lambda*4*-benzo[1,4]oxathiin-2-ylmethyl]-isoindole-1,3-dione (0.040 g, 0.082 mmol) was dissolved in MeOH (4 mL) and hydrazine hydrate (0.012 mL, 0.246 mmol) was added dropwise. The reaction mixture was heated at reflux for 5 hours. The solvent was evaporated and the crude was dissolved in DCM (3 mL). A 1 M solution of HCl in Et₂O was added and the reaction mixture was stirred at room temperature for 30 minutes. The solvent was removed under vacuum and the crude was suspended in AcOEt and washed twice with a satured aqueous solution of NaHCO₃ (3 mL). The aqueous layer was extracted once with DCM and the collected organics were dried over anhydrous sodium sulphate, filtered and evaporated under vacuum. Purification by flash column chromatography, (Silica, DCM to DCM:MeOH 9:1) afforded the pure title compound as the free base. A solution of gaseous HCl in Et₂O was added and the mixture was stirred at room temperature for 10 minutes. The solvent was removed under vacuum to afford the title compound as hydrochloric salt (0.020 g, 62.3% yield). MS (ESI) m/z 359.9/361.9.0 [M+H]⁺.

¹H NMR (300 MHz, DMSO-d6+TFA) d ppm 8.05 (br. s., 3H) 7.88 (dd, 1H) 7.55-7.62 (m, 2H) 7.41-7.52 (m, 2H) 4.75-4.85 (m, 1H) 3.53 (d, 1H) 3.09-3.33 (m, 2H) 3.15 (dd, 1H).

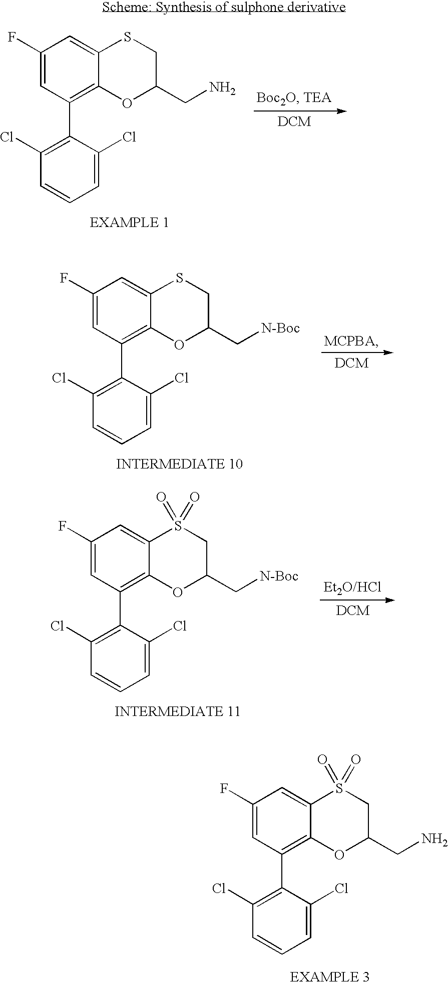

EXAMPLE 1 was obtained as previously described.

### Intermediate 10

[8-(2,6-Dichloro-phenyl)-6-fluoro-2,3-dihydro-benzo[1,4]oxathiin-2-ylmethyl]-carbamic acid tert-butyl ester: To a solution of C-[8-(2,6-Dichloro-phenyl)-6-fluoro-2,3-dihydro-benzo[1,4]oxathiin-2-yl]-methylamine (0.04 g, 0.12 mmol) and TEA (25 μl 0.18 mmol) in DCM (2 mL) was added a solution of Boc₂O (0.036 g, 0.17 mmol) in DCM (0.5 mL). The solution was stirred at room temperature for 16 h. The mixture was diluted with DCM and washed with citric acid 5% (3×2 mL), NaHCO₃ 5% (3×2 mL) and brine. The organic layer was separated, dried (sodium sulfate), filtered and the solvent was removed under vacuum. Purification by filtration using Silica (5 g) IST cartridge (Hexane:AcOEt 9:1) afforded 0.045 g of pure title compound (85% yield). MS (ESI) m/z 343.8/375.8 [M+H]⁺.

### Intermediate 11

[8-(2,6-Dichloro-phenyl)-6-fluoro-4,4-dioxo-3,4-dihydro-2H-4lambda*6*-benzo[1,4]oxathiin-2-ylmethyl]-carbamic acid tert-butyl ester: A solution of [8-(2,6-Dichloro-phenyl)-6-fluoro-2,3-dihydro-benzo[1,4]oxathiin-2-ylmethyl]-carbamic acid tert-butyl ester (0.10 mmol, 0.045 g) in anhydrous DCM (2 mL) was cooled to 0° C. A solution of MCPBA (77%, 0.056 g, 0.25 mmol) in anhydrous DCM (2 mL) was added under inert atmosphere. The reaction mixture was stirred at 0° C. for 1 h and at room temperature for 1.5 h. Aqueous Na₂SO₃ (10%, 2 mL) was added at 0° C. and the reaction mixture was stirred for 15′. The organic layer was separated and washed with NaHCO₃ 5% (3×2 mL) and brine. The organic phase was separated, dried (sodium sulfate), filtered and the solvent was removed under vacuum. Purification by filtration using Silica (5 g) IST cartridge (Hexane:AcOEt from 8:2 to 7:3) afforded 0.042 g of pure title compound (88% yield). MS (ESI) m/z 375.8/377.8 [M+H] as the major fragment (loss of Boc fragment).

### Example 3

C-[8-(2,6-Dichloro-phenyl)-6-fluoro-4,4-dioxo-3,4-dihydro-2H-4lambda*6*-benzo[1,4]oxathiin-2-yl]-methylamine (hydrochloride salt): [8-(2,6-Dichloro-phenyl-6-fluoro-4,-dioxo-3,4-dihydro-2H-4lambda*6*-benzo[1,4]oxathiin-2-ylmethyl]-carbamic acid tert-butyl ester (0.042 g, 0.112 mmol) was dissolved in DCM (5 mL) and gaseous HCl in Et₂O (large excess) was added at 0° C. The reaction mixture was stirred at room temperature for 48 h. The solvent was removed under vacuum and the white solid that formed was triturated with DCM/Et₂O. 0.020 g (54% yield) of pure title compound were obtained as hydrochloride salt. MS (ESI) m/z 376.02/378.04 [M+H]⁺.

¹H NMR (300 MHz, DMSO-d6) d ppm 7.85 (dd, 1H) 7.59-7.64 (m, 3H) 7.47-7.55 (m, 1H) 7.17 (br. s., 2H) 4.83-4.96 (m, 1H) 4.12 (d, 1H) 3.93 (dd, 1H) 2.99-3.15 (m, 2H).

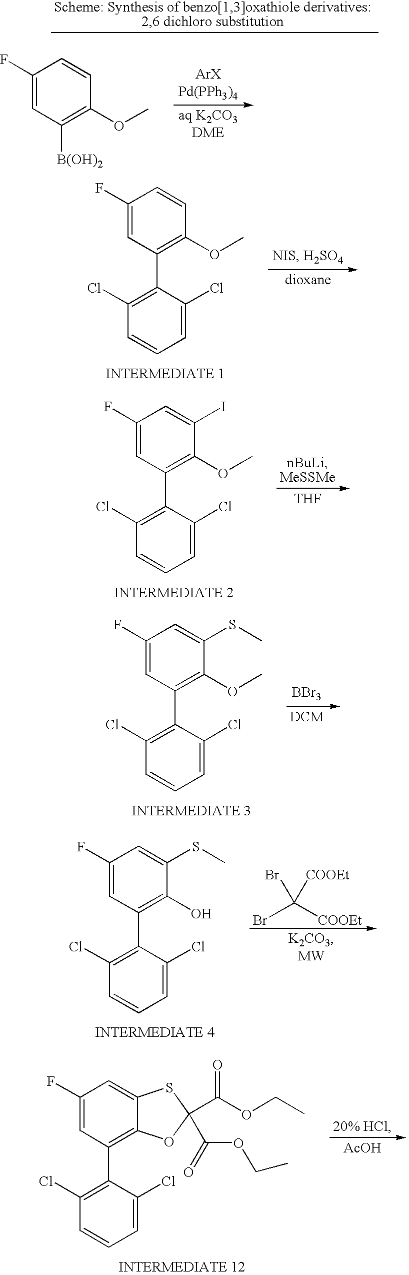

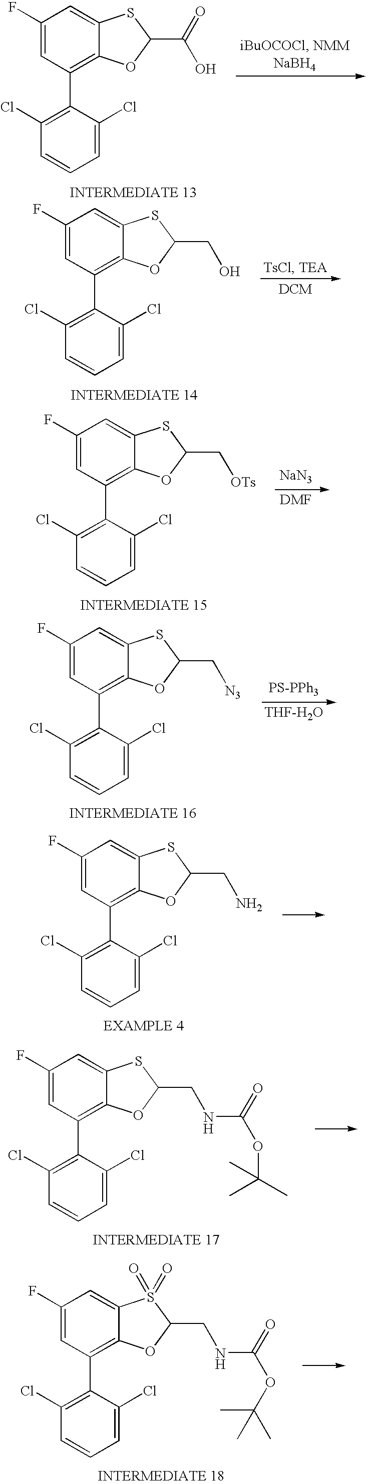

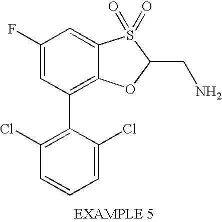

INTERMEDIATES 1, 2, 3 and 4 have been obtained as previously described

### Intermediate 12

7-(2,6-Dichloro-phenyl)-5-fluoro-benzo[1,3]oxathiole-2,2-dicarboxylic acid diethyl ester: 2′,6′-Dichloro-5-fluoro-3-methylsulfanyl-biphenyl-2-ol (0.99 mmol, 0.300 g) was dissolved in 2,2-Dibromo-malonic acid diethyl ester (2.0 mL). Solid K₂CO₃ (1.98 mmol, 0.273 g) and 4 Å Molecular sieves were added and the reaction mixture was heated at 140° C. under microwave irradiation for 30 minutes. Molecular sieves were filtered off and the mixture was diluted with water (2 mL) and extracted with ethyl ether (3×5 mL). The combined organics were dried (sodium sulfate) and evaporated under vacuum. Purification by flash chromatography, (Silica, Hexanes to Hexanes:AcOEt 8:2) afforded 120 mg of pure title compound (30% yield). MS (ESI) m/z 445.0/447.0 [M+H]⁺.

¹H NMR (300 MHz, DMSO-d6, 373 K) d ppm 7.52-7.59 (m, 2H) 7.46 (dd, 1H) 7.34 (dd, 1H) 6.82 (dd, 1H) 4.27 (q, 2H) 1.22 (t, 6H).

### Intermediate 13

7-(2,6-Dichloro-phenyl)-5-fluoro-benzo[1,3]oxathiole-2-carboxylic acid: 7-(2,6-Dichloro-phenyl)-5-fluoro-benzo[1,3]oxathiole-2,2-dicarboxylic acid diethyl ester (0.03 g, 0.067 mmol) was dissolved a mixture of 20% solution of aqueous HCl and acetic acid (2.5 mL:2.5 mL). The reaction mixture was heated at 100° C. for 6 h then cooled to room temperature. Water (4 mL) was added and the reaction was extracted with DCM (3×4 mL). The collected organic layers were dried (sodium sulfate), filtered and evaporated under vacuum. The crude was further dried in vacuo at 40° C. for 16 h. 0.023 g of title compound (quantitative yield) were recovered and submitted to the following step without further purification. MS (ESI) m/z 299.0/301.0 [M+H]⁺ as the major fragment (loss of COOH fragment).

¹H NMR (300 MHz, DMSO-d6) d ppm 13.58 (br. s., 1H) 7.53-7.61 (m, 2H) 7.46 (dd, 1H) 7.36 (dd, 1H) 6.84 (dd, 1H) 6.53 (s, 1H).

### Intermediate 14

[7-(2,6-Dichloro-phenyl)-5-fluoro-benzo[1,3]oxathiol-2-yl]methanol: To a solution of 7-(2,6-Dichloro-phenyl)-5-fluoro-benzo[1,3]oxathiole-2-carboxylic acid (540 mg, 1.56 mmol) in DME (20 mL), under inert atmosphere, were added isobutyl chloroformate (0.20 mL, 1.56 mmol) and NMM (0.16 mL, 1.56 mmol). The reaction was stirred at room temperature for 2 h. The solid was filtered off under vacuum, and the solution was directly poured into a round bottom flask kept under inert atmosphere. NaBH₄ (118 mg, 3.13 mmol) was added and the reaction was stirred at room temperature for 4 h. DCM was added and the solution was washed with aq. NH₄Cl solution (1×15 mL) and brine. The organic layer was dried (sodium sulfate), filtered and evaporated under vacuum. Purification by flash chromatography (Silica, DCM) afforded 230 mg of pure title compound (50% yield over two steps). ¹H NMR (300 MHz, DMSO-d6, 373 K) d ppm 7.49-7.57 (m, 2H), 7.43 (dd, 1H), 7.20 (dd, 1H), 6.67 (dd, 1H), 6.11 (t, 1H), 4.82 (br. s., 1H), 3.79 (dd, 1H), 3.64 (dd, 1H).

### Intermediate 15

Toluene-4-sulfonic acid 5-fluoro-7-(2,6-Dichloro-phenyl)-benzo[1,3]oxathiol-2-ylmethyl ester: A solution of [5-Fluoro-7-(2,6-Dichloro-phenyl)-benzo[1,3]oxathiol-2-yl]-methanol (0.230 g, 0.70 mmol) and TEA (0.290 mL, 2.10 mmol) in DCM (15 mL) was cooled at 0° C. and p-toluenesulfonyl chloride (0.390 g, 2.10 mmol) was added. The reaction mixture was stirred at RT for 12 h and then diluted with methylene chloride (50 mL), washed with 1N HCl solution (1×30 mL) and brine. The organic layer was dried (sodium sulfate), filtered and evaporated under vacuum. Purification by flash chromatography, (Silica, Hexanes:DCM 1:1) afforded 0.145 g of pure title compound (43% yield). ¹H NMR (300 MHz, DMSO-d6) d ppm 7.53-7.58 (m, 2H), 7.47-7.52 (m, 2H), 7.32-7.46 (m, 1H), 7.07-7.18 (m, 2H), 7.03 (dd, 1H), 6.70 (dd, 1H), 5.46 (dd, 1H), 3.11 (dd, 1H), 2.96 (dd, 1H), 2.31 (s, 3H).

### Intermediate 16

2-Azidomethyl-5-fluoro-7-(2,6-Dichloro-phenyl)-benzo[1,3]oxathiole: To a solution of toluene-4-sulfonic acid 5-fluoro-7-(2,6-Dichloro-phenyl)-benzo[1,3]oxathiol-2-ylmethyl ester (0.220 g, 0.45 mmol) in DMF (2.5 mL), NaN₃ (0.140 g, 2.26 mmol) was added under inert atmosphere and the reaction mixture was allowed to stir at 90° C. for 16 h. The solvent was concentrated under vacuum and the residue portioned between brine and Et₂O. The organic layer was dried (sodium sulfate), filtered and concentrated under vacuum. The product obtained was used as such in the next step. MS (ESI) m/z 329.9/331.9 [M+H]⁺ as the major fragment (loss of N₂ fragment).

### Example 4

C-[5-Fluoro-7-(2,6-Dichloro-phenyl)-benzo[1,3]oxathiol-2-yl]-methylamine (hydrochloric salt): To a suspension of swelled PS-PPh₃ (0.520 g, 1.35 mmol, loading 2.6 mmol/g) in THF (10 mL), a solution of 2-Azidomethyl-5-fluoro-7-(2,6-Dichloro-phenyl)-benzo[1,3]oxathiole (theoretical 0.45 mmol) in THF (2 mL) was added. The reaction was allowed to shake at room temperature for 20 h. H₂O (2 mL) was added to the mixture and the reaction was shaked for additional 24 h. The resin was filtered off and washed with DCM. The solution was loaded on a strong cation exchange resin and the product was eluted with a 3% solution of NH₄OH in MeOH. The solvent was removed under vacuum and the residue dissolved in DCM. A solution of gaseous HCl in Et₂O was added and the solution was stirred at room temperature for 10 minutes. The solvent was removed under vacuum to afford 65 mg of pure title compound as hydrochloric salt (37% yield over two steps). MS (ESI) m/z 329.99 [M+H]⁺¹H NMR (300 MHz, DMSO-d6, 373 K) d ppm 7.53-7.59 (m, 2H), 7.40-7.49 (m, 1H), 7.32 (dd, 1H), 6.77 (dd, 1H), 6.41 (dd, 1H), 3.34 (dd, 1H), 3.27 (dd, 1H).

### Intermediate 17

[7-(2,6-Dichloro-phenyl)-5-fluoro-benzo[1,3]oxathiol-2-ylmethyl]-carbamic acid tert-butyl ester: To a solution of C-[5-Fluoro-7-(2,6-dichloro-phenyl)-benzo[1,3]oxathiol-2-yl]-methylamine (0.09 g, 0.27 mmol) and TEA (57 μl, 0.4 mmol) in DCM (4 mL) was added a solution of Boc₂O (0.036 g, 0.17 mmol) in DCM (1 mL). The solution was stirred at room temperature for 16 h. The mixture was diluted with DCM and washed with citric acid 5% (3×3 mL), NaHCO₃ 5% (3×3 mL) and brine. The organic layer was separated, dried (sodium sulfate), filtered and the solvent was removed under vacuum. 81 mg of title compound were obtained and were used as such in the following step. MS (ESI) m/z 330.0/332.0 [M+H]⁺ as the fragment (loss of Boc fragment) and m/z 312.9/314.9 [M+H]⁺ as the major fragment (elimination of the side chain).

### Intermediate 18

[7-(2,6-Dichloro-phenyl)-5-fluoro-3,3-dioxo-2,3-dihydro-3lambda*6*-benzo[1,3]oxathiol-2-ylmethyl]-carbamic acid tert-butyl ester: A solution of [7-(2,6-Dichloro-phenyl)-5-fluoro-benzo[1,3]oxathiol-2-ylmethyl]-carbamic acid tert-butyl ester (0.09 mmol, 0.037 g) in anhydrous DCM (4 mL) was cooled to 0° C. A solution of MCPBA (77%, 0.112 g, 0.5 mmol) in anhydrous DCM (4 mL) was added under inert atmosphere. The reaction mixture was stirred at 0° C. for 1 h and at room temperature for 1.5 h. Aqueous Na₂SO₃ (10%, 3 mL) was added at 0° C. and the reaction mixture was stirred for 15′. The organic layer was separated and washed with NaHCO₃ 5% (3×3 mL) and brine. The organic phase was separated, dried (sodium sulfate), filtered and the solvent was removed under vacuum. Purification by filtration using Silica (5 g) IST cartridge (Hexane:AcOEt from 8:2 to 7:3) afforded 0.041 g of pure title compound (quantitative yield). MS (ESI) m/z 361.9/363.9 [M+H]⁺ as the fragment (loss of Boc fragment).

### Example 5

C-[7-(2,6-Dichloro-phenyl)-5-fluoro-3,3-dioxo-2,3-dihydro-3lambda*6*-benzo[1,3]oxathiol-2-yl]-methylamine (Hydrochloric salt): [8-(2,6-Dichloro-phenyl-6-fluoro-4-dioxo-3,4-dihydro-2H-4lambda*6*-benzo[1,4]oxathiin-2-ylmethyl]-carbamic acid tert-butyl ester (0.041 g, 0.09 mmol) was dissolved in DCM (3 mL) and trifluoroacetic acid (0.1 mL) was added at 0° C. The reaction mixture was stirred at room temperature for 3 h. The solvent was removed under vacuum and the solid that formed was dissolved in a 1:1 DCM:MeOH mixture: The solution was loaded on a strong cation exchange resin and the product was eluted with a 3% solution of NH₄OH in MeOH. The solvent was removed under vacuum and the residue dissolved in DCM. A solution of gaseous HCl in Et₂O was added and the solution was stirred at room temperature for 10′. The solvent was removed under vacuum to afford the title compound as hydrochloric salt (27 mg, 75% yield). MS (ESI) m/z 361.95 [M+H]⁺. ′1H NMR (300 MHz, DMSO-d6) d ppm 7.97 (dd, 1H), 7.58-7.67 (m, 3H), 7.48-7.58 (m, 1H), 5.69 (dd, 0H), 3.55 (dd, 1H), 3.23 (dd, 1H).

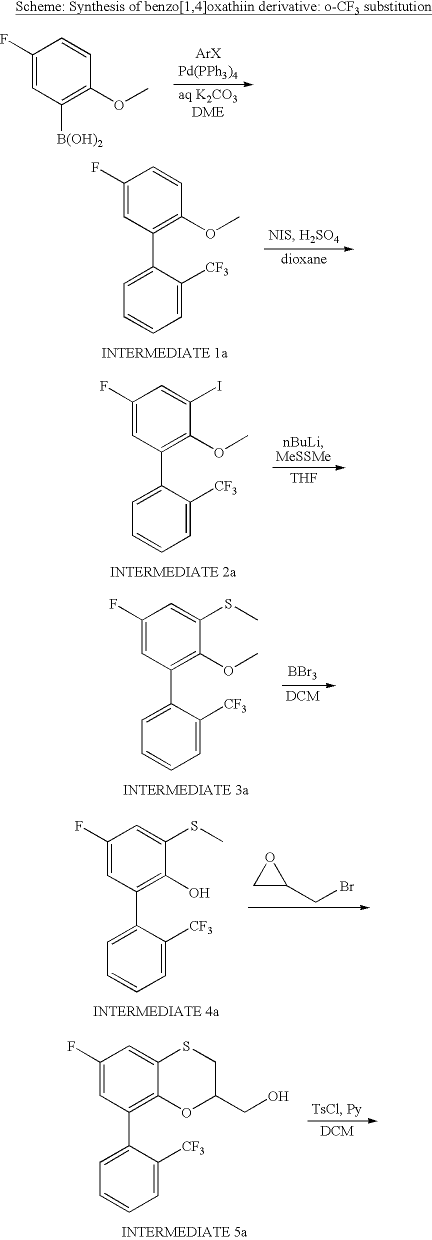

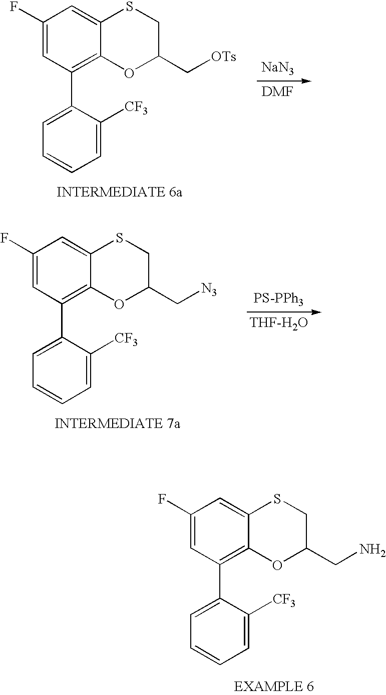

### Intermediate 1a

5-Fluoro-2-methoxy-2′-trifluoromethyl-biphenyl: Prepared according to the procedure described for INTERMEDIATE 1 starting from 2-methoxy-5-fluorophenylboronic acid (25.0 g, 147.1 mmol) and 1-Bromo-2-trifluoromethyl-benzene (19.9 g, 88.4 mmol). 14.1 g (59%) of the title compound as light yellow oil were obtained. ¹H NMR (300 MHz, DMSO-d6) d ppm 7.79 (dd, 1H), 7.65-7.74 (m, 1H), 7.55-7.65 (m, 1H), 7.30-7.38 (m, 1H), 7.23 (ddd, 1H), 7.09 (dd, 1H), 7.00 (dd, 1H), 3.65 (s, 3H).

### Intermediate 2a

5-Fluoro-3-iodo-2-methoxy-2′-trifluoromethyl-biphenyl: Prepared according to the procedure described for INTERMEDIATE 2 starting from 5-Fluoro-2-methoxy-2′-trifluoromethyl-biphenyl (14.024 g, 0.051 mol). Purification by flash chromatography (Silica, Hexanes:Acetone 99:1 to 95:5) afforded 15 g (74%) of the title compound. MS (ESI) m/z 396.0 [M+H]⁺. ¹H NMR (300 MHz, DMSO-d6 373 K) d ppm 7.80-7.86 (m, 1H), 7.65-7.74 (m, 3H), 7.42-7.50 (m, 1H), 7.03-7.09 (m, 1H), 3.37 (s, 3H).

### Intermediate 3a

5-Fluoro-2-methoxy-3-methylsulfanyl-2′-trifluoromethyl-biphenyl: Prepared according to the procedure described for INTERMEDIATE 3 starting from 5-Fluoro-3-iodo-2-methoxy-2′-trifluoromethyl-biphenyl (5.0 g, 12.6 mmol). Purification by flash chromatography (Silica, Hexanes to Hexanes: DCM 85:15) afforded 2.35 g (59%) of the title compound. MS (ESI) m/z 317.0 [M+H]⁺; MS (ESI) m/z 333.0 [M+H]⁺ also detected as the major peak. ¹H NMR (300 MHz, DMSO-d6 373 K) d ppm 7.82 (d, 1H), 7.54-7.79 (m, 2H), 7.44 (d, 1H), 7.12 (dd, 1H), 6.77 (dd, 1H), 3.38 (s, 3H), 2.49 (s, 3H)

### Intermediate 4a

5-Fluoro-3-methylsulfanyl-2′-trifluoromethyl-biphenyl-2-ol: Prepared according to the procedure described for INTERMEDIATE 4 starting from 5-Fluoro-2-methoxy-3-methylsulfanyl-2′-trifluoromethyl-biphenyl (4.4 g, 13.9 mmol). Purification by flash chromatography (Silica, Hexanes:AcOEt 99:51 to 95:5) afforded 2.4 g (55%) of pure title compound. MS (ESI) m/z 302.0 [M+H]⁺. ¹H NMR (300 MHz, DMSO-d6) d ppm 8.55 (s, 1H), 7.80 (dd, 1H), 7.51-7.73 (m, 2H), 7.34 (d, 1H), 7.03 (dd, 1H), 6.68 (dd, 1H), 2.42 (s, 3H).

### Intermediate 5a

[6-Fluoro-8-(2-trifluoromethyl-phenyl)-2,3-dihydro-benzo[1,4]oxathiin-2-yl]-methanol: Prepared according to the procedure described for INTERMEDIATE 5 starting from 5-Fluoro-3-methylsulfanyl-2′-trifluoromethyl-biphenyl-2-ol (0.600 g, 1.90 mmol,). Purification by flash chromatography (Silica, Hexanes:AcOEt 7:3) afforded 0.43 g (62%) of pure title compound. MS (ESI) m/z 345.0 [M+H]⁺. ¹H NMR (300 MHz, DMSO-d6 +TFA) d ppm 7.74-7.79 (m, 1H), 7.54-7.71 (m, 2H), 7.34 (d, 1H), 7.00 (dd, 1H), 6.68 (ddd, 1H), 3.97-4.12 (m, 1H), 3.47 (dd, 1H), 3.38 (dd, 1H), 3.19 (dd, 1H), 3.03 (dd, 1H).

### Intermediate 6a

Toluene-4-sulfonic acid 6-fluoro-8-(2-trifluoromethyl-phenyl)-2,3-dihydro-benzo[1,4]oxathiin-2-ylmethyl ester: Prepared according to the procedure described for INTERMEDIATE 6 starting from [6-Fluoro-8-(2-trifluoromethyl-phenyl)-2,3-dihydro-benzo[1,4]oxathiin-2-yl]-methanol (0.546 g, 1.59 mmol). Purification by flash chromatography (Silica, Hexanes:DCM 1:1) afforded 0.31 g (40%) of pure title compound. MS (ESI) m/z 499.0 [M+H]⁺ 1H NMR (300 MHz, DMSO-d6+TFA) d ppm 7.75 (dd, 1H), 7.51-7.70 (m, 4H), 7.37-7.44 (m, 2H), 7.28-7.34 (m, 1H), 7.02 (dd, 1H), 6.70 (ddd, 1H), 4.23-4.46 (m, 1H), 4.13 (dd, 1H), 3.97-4.07 (m, 1H), 3.16 (dd, 1H), 3.01 (dd, 1H), 2.44 (s, 3H).

### Intermediate 7a

2-Azidomethyl-6-fluoro-8-(2-trifluoromethyl-phenyl)-2,3-dihydro-benzo[1,4]oxathiine: Prepared according to the procedure described for INTERMEDIATE 7 starting from toluene-4-sulfonic acid 6-fluoro-8-(2-trifluoromethyl-phenyl)-2,3-dihydro-benzo[1,4]oxathiin-2-ylmethyl ester (0.309 g, 0.2620 mmol). The product obtained was used as such in the next step. MS (ESI) m/z 342.0 [M+H]⁺ as the major fragment (loss of N₂).

### Example 6

C-[6-Fluoro-8-(2-trifluoromethyl-phenyl)-2,3-dihydro-benzo[1,4]oxathiin-2-yl]-methylamine (hydrochloric salt): Prepared according to the procedure described for EXAMPLES 1 and 4 starting from 2-Azidomethyl-6-fluoro-8-(2-trifluoromethyl-phenyl)-2,3-dihydro-benzo[1,4]oxathiine (theoretical 0.62 mmol). 130 mg of pure title compound (64% over two steps) were obtained as hydrochloric salt. MS (ESI) m/z 344.08 [M+H]⁺. ¹H NMR (300 MHz, DMSO-d6) d ppm 7.75-7.84 (m, 1H), 7.55-7.74 (m, 2H), 7.37-7.47 (m, 1H), 7.07 (dd, 1H), 6.73 (dd, 1H), 4.46 (br. s., 1H), 2.71-3.41 (m, 4H).

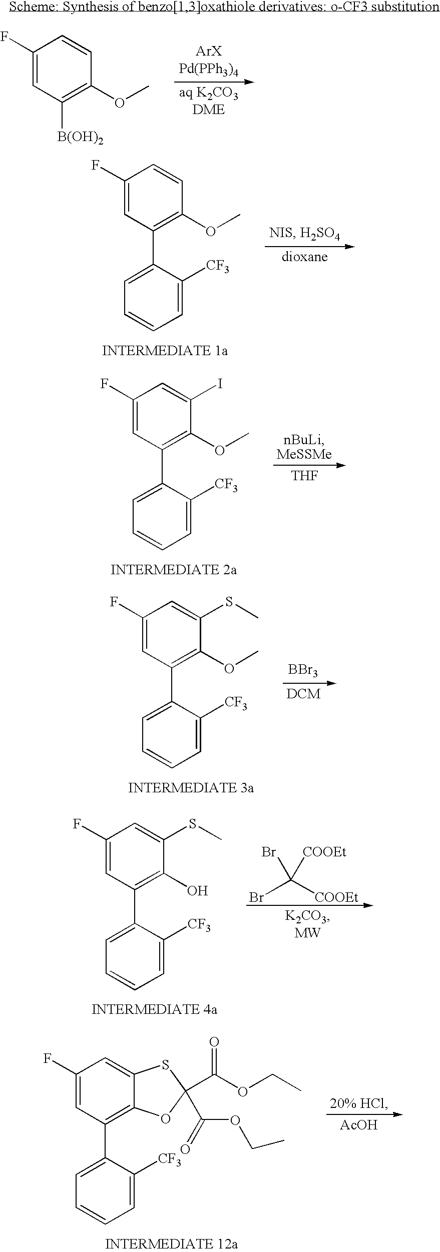

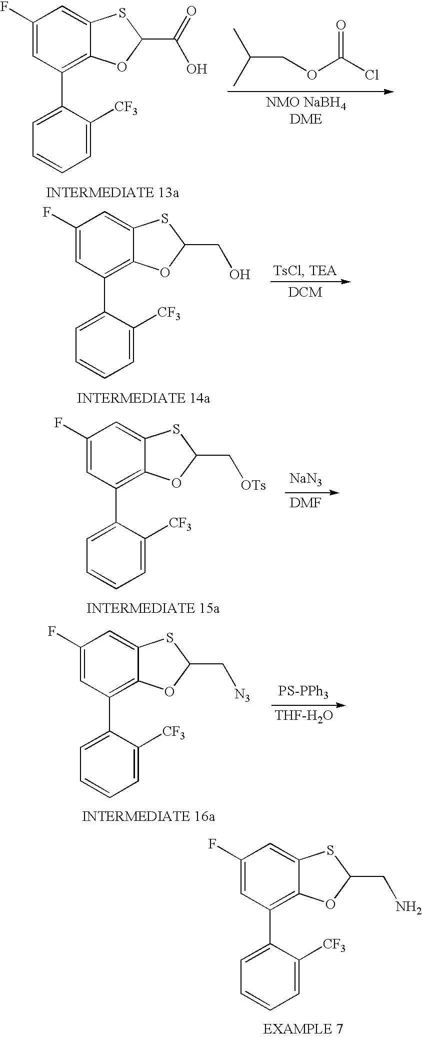

INTERMEDIATES 1a, 2a, 3a and 4a have been obtained as previously described

### Intermediate 12a

5-fluoro7-(2-trifluoromethyl-phenyl)-benzo[1,3]oxathiole-2,2-dicarboxylic acid diethyl ester: Prepared according to the procedure described for INTERMEDIATE 12 starting from 5-Fluoro-3-methylsulfanyl-2′-trifluoromethyl-biphenyl-2-ol (1.23 mmol, 0.370 g). Purification by flash chromatography (Silica, Hexanes to Hexanes:AcOEt 8:2) afforded 0.18 g (33%) of pure title compound. MS (ESI) m/z 445 [M+H]⁺.

### Intermediate 13a

5-Fluoro-7-(2-trifluoromethyl-phenyl)-benzo[1,3]oxathiole-2-carboxylic acid: Prepared according to the procedure described for INTERMEDIATE 13 starting from 5-fluoro7-(2-trifluoromethyl-phenyl)-benzo[1,3]oxathiole-2,2-dicarboxylic acid diethyl ester (0.563 g, 1.268 mmol). The product obtained was used as such in the next step. MS (ESI) m/z 299.0 [M+H]⁺ as the major fragment (loss of COOH fragment).

### Intermediate 14a

[5-Fluoro-7-(2-trifluoromethyl-phenyl)-benzo[1,3]oxathiol-2-yl]methanol: Prepared according to the procedure described for INTERMEDIATE 14 starting from 5-Fluoro-7-(2-trifluoromethyl-phenyl)-benzo[1,3]oxathiole-2-carboxylic acid (0.41 mg, 1.19 mmol). Purification by flash chromatography (Silica, DCM) afforded 0.13 g (33%) of pure title compound. MS (ESI) m/z 330.0 [M+H]⁺. 1H NMR (300 MHz, DMSO-d6 373 K) d ppm 7.76-7.84 (m, 1H), 7.55-7.75 (m, 2H), 7.36-7.47 (m, 1H), 7.17 (dd, 1H), 6.64 (dd, 1H), 6.07 (t, 1H), 4.72-4.95 (m, 1H), 3.52-3.83 (m, 2H).

### Intermediate 15a

Toluene-4-sulfonic acid 5-fluoro-7-(2-trifluoromethyl-phenyl)-benzo[1,3]oxathiol-2-ylmethyl ester: Prepared according to the procedure described for INTERMEDIATE 14 starting from [5-Fluoro-7-(2-trifluoromethyl-phenyl)-benzo[1,3]oxathiol-2-yl]-methanol (0.130 g, 0.39 mmol). Purification by flash chromatography (Silica, Hexanes:DCM 1:1) afforded 0.098 g (52%) of pure title compound. MS (ESI) m/z 485.0 [M+H]⁺.

### Intermediate 16a

2-Azidomethyl-5-fluoro-7-(2-trifluoromethyl-phenyl)-benzo[1,3]oxathiole: Prepared according to the procedure described for INTERMEDIATE 16 starting from toluene-4-sulfonic acid 5-fluoro-7-(2-trifluoromethyl-phenyl)-benzo[1,3]oxathiol-2-ylmethyl ester (0.098 g, 0.20 mmol). The product obtained was used as such in the next step. MS (ESI) m/z 330.0 [M+H]⁺ as the major fragment (loss of N₂ fragment).

### Example 7

C-[5-Fluoro-7-(2-trifluoromethyl-phenyl)-benzo[1,3]oxathiol-2-yl]methylamine (hydrochloric salt): Prepared according to the procedure described for EXAMPLES 1, 4 and 6 starting from 2-Azidomethyl-5-fluoro-7-(2-trifluoromethyl-phenyl)-benzo[1,3]oxathiole (theoretical 0.20 mmol). 28 mg of pure title compound (43% over two steps) were obtained as hydrochloric salt. MS (ESI) m/z 330.08 [M+H]⁺. ¹H NMR (300 MHz, DMSO-d6 373K) d ppm 7.82 (dd, 1H), 7.59-7.78 (m, 2H), 7.42-7.53 (m, 1H), 7.29 (dd, 1H), 6.73 (dd, 1H), 6.36 (t, 1H), 3.27-3.36 (m, 2H)

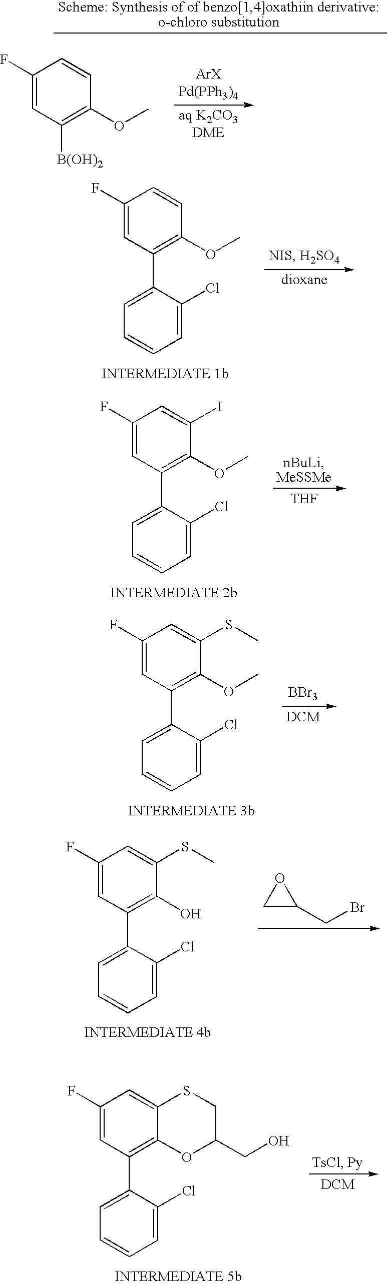

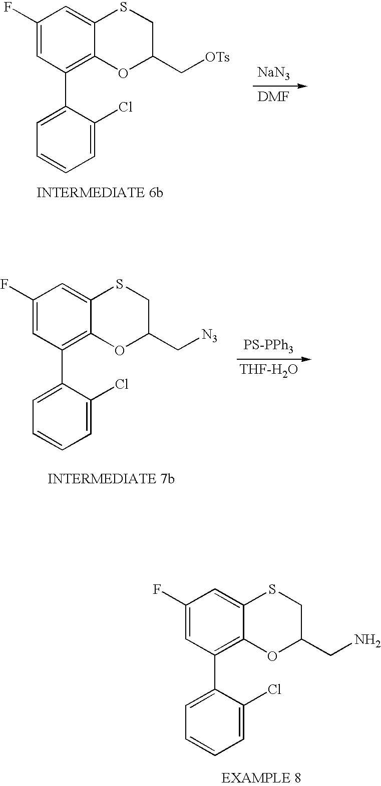

### Intermediate 1b

5-Fluoro-2-methoxy-2′-chloro-biphenyl: Prepared according to the procedure described for INTERMEDIATE 1 starting from 2-methoxy-5-fluorophenylboronic acid (50.0 g, 294.2 mmol) and 1-Bromo-2-chloro-benzene (33.8 g, 177.2 mmol). 40.0 g (95%) of the title compound as colourless oil were obtained. ′1H NMR (300 MHz, DMSO-d6) d ppm 7.46-7.56 (m, 1H), 7.26-7.43 (m, 3H), 7.18 (ddd, 1H), 7.11 (dd, 1H), 6.97 (dd, 1H), 3.72 (s, 3H).

### Intermediate 2b

5-Fluoro-3-iodo-2-methoxy-2′-chloro-biphenyl: Prepared according to the procedure described for INTERMEDIATE 2 starting from 5-Fluoro-2-methoxy-2′-chloro (20 g, 0.085 mol). Purification by flash chromatography (Silica, Hexanes:Acetone 99:1 to 95:5) afforded 17.6 g of a mixture of the title compound and starting material. The mixture was used as such in the following step. MS (ESI) m/z 362.0/364.0 [M+H]⁺.

### Intermediate 3b

5-Fluoro-2-methoxy-3-methylsulfanyl-2′-chloro-biphenyl: Prepared according to the procedure described for INTERMEDIATE 3 starting from 5-Fluoro-3-iodo-2-methoxy-2′-chloro-biphenyl (5.0 g, 13.8 mmol). Purification by flash chromatography (Silica, Hexanes to Hexanes: DCM 85:15) afforded 2.06 g (53%) of the title compound. MS (ESI) m/z 283.0/285.0 [M+H]⁺. ′1H NMR (300 MHz, DMSO-d6 373 K) d ppm 7.49-7.60 (m, 1H), 7.28-7.48 (m, 3H), 7.10 (dd, 1H), 6.79 (dd, 1H), 3.39 (s, 3H), 2.49 (s, 3H).

### Intermediate 4b

5-Fluoro-3-methylsulfanyl-2′-chloro-biphenyl-2-ol: Prepared according to the procedure described for INTERMEDIATE 4 starting from 5-Fluoro-2-methoxy-3-methylsulfanyl-2′-chloro-biphenyl (4.93 g, 17 mmol). Purification by flash chromatography (Silica, Hexanes:AcOEt 99:51 to 95:5) afforded 2.22 g (48%) of pure title compound. MS (ESI) m/z 269.0/271.0 [M+H]+. ′1H NMR (300 MHz, DMSO-d6 353 K) d ppm 8.23 (s, 1H), 7.46-7.59 (m, 1H), 7.24-7.45 (m, 3H), 7.07 (dd, 1H), 6.74 (dd, 1H), 2.44 (s, 3H).

### Intermediate 5b

[6-Fluoro-8-(2-chloro-phenyl)-2,3-dihydro-benzo[1,4]oxathiin-2-yl]-methanol: Prepared according to the procedure described for INTERMEDIATE 5 starting from 5-Fluoro-3-methylsulfanyl-2′-chloro-biphenyl-2-ol (1.015 g, 3.4 mmol). Purification by flash chromatography (Silica, Hexanes:AcOEt 7:3) afforded 0.71 g (61%) of pure title compound. MS (ESI) m/z 293.0/295.0 [M+H]⁺ as the major fragment (loss of H₂O).

### Intermediate 6b

Toluene-4-sulfonic acid 6-fluoro-8-(2-chloro-phenyl)-2,3-dihydro-benzo[1,4]oxathiin-2-ylmethyl ester: Prepared according to the procedure described for INTERMEDIATE 6 starting from [6-Fluoro-8-(2-chloro-phenyl)-2,3-dihydro-benzo[1,4]oxathiin-2-yl]-methanol (0.71 g, 2.28 mmol). Purification by flash chromatography (Silica, Hexanes:DCM 1:1) afforded 0.5 g (47%) of pure title compound. MS (ESI) m/z 293.0/295.0 [M+H]⁺ as the major fragment (loss of Tosyl-fragment). ′1H NMR (300 MHz, DMSO-d6 353 K) d ppm 7.63 (m, 2H), 7.22-7.54 (m, 6H), 7.04 (dd, 1H), 6.75 (dd, 1H), 4.33-4.50 (m, 1H), 4.20 (dd, 1H), 4.09 (dd, 1H), 3.18 (dd, 1H), 3.05 (dd, 1H), 2.44 (s, 3H).

### Intermediate 7b

2-Azidomethyl-6-fluoro-8-(2-chloro-phenyl)-2,3-dihydro-benzo[1,4]oxathiine: Prepared according to the procedure described for INTERMEDIATE 7 starting from toluene-4-sulfonic acid 6-fluoro-8-(2-chloro-phenyl)-2,3-dihydro-benzo[1,4]oxathiin-2-ylmethyl ester (0.5 g, 1.08 mmol). The product obtained was used as such in the next step. MS (ESI) m/z 308.0/310.0 [M+H]⁺ as the major fragment (loss of N₂).

### Example 8

C-[6-Fluoro-8-(2-chloro-phenyl)-2,3-dihydro-benzo[1,4]oxathiin-2-yl]-methylamine (hydrochloric salt): Prepared according to the procedure described for EXAMPLES 1, 4, and 7 starting from 2-Azidomethyl-6-fluoro-8-(2-chloro-phenyl)-2,3-dihydro-benzo[1,4]oxathiine (theoretical 1.08 mmol). 279 mg of pure title compound (84% over two steps) were obtained as hydrochloric salt. MS (ESI) m/z 310.0/312.0 [M+H]⁺. ¹H NMR (300 MHz, DMSO-d6, 353K+TFA) d ppm 7.95 (br. s., 3H), 7.49-7.56 (m, 1H), 7.36-7.46 (m, 3H), 7.10 (dd, 1H), 6.79 (dd, 1H), 4.45-4.56 (m., 1H), 3.33 (dd, 1H), 3.18 (dd, 1H), 3.13 (dd, 1H), 3.05 (dd, 1H).

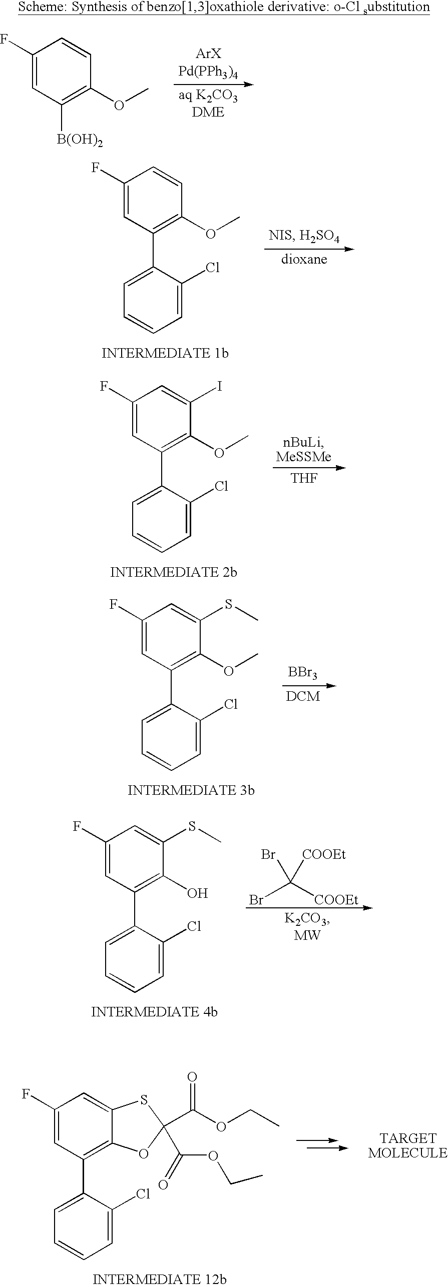

### Intermediates 1b, 2b, 3b and 4b have been Obtained as Previously Described

### Intermediate 12b

5-fluoro7-(2-chloro-phenyl)-benzo[1,3]oxathiole-2,2-dicarboxylic acid diethyl ester: Prepared according to the procedure described for INTERMEDIATE 12 starting from 5-Fluoro-3-methylsulfanyl-2′-chloro-biphenyl-2-ol (1.100 g, 4.1 mmol). Purification by flash chromatography (Silica, Hexanes to Hexanes:AcOEt 8:2) afforded 0.49 g (29%) of title compound. MS (ESI) m/z 411.0/413.0 [M+H]⁺.

### BIOLOGICAL ASSAYS

The compounds of this invention are agonists and partial agonists at the 2C subtype of brain serotonin receptors and are thus of interest for the treatment of schizophrenia and related disorders such as schizoaffective disorder, schizophreniform disorder, L-DOPA-induced psychosis and bipolar disorder, depression, including related disorders such as obsessive compulsive disorder and panic disorder, and obesity, with its consequent comorbidities including Type II diabetes, cardiovascular disease, hypertension, hyperlipidemia, stroke, osteoarthritis, sleep apnea, gall bladder disease, gout, some cancers, some infertility, and early mortality. These, and other disorders the treatment of which the present compounds are useful, are discussed herein.

### A. Assessment of Effectiveness of Compounds as 5HT_{2C} Agonists and Partial Agonists

The ability of the compounds of this invention to act as 5HT_{2C} agonists and partial agonists was established using several standard pharmacological test procedures; the procedures used and results obtained are provided below. In the test procedures, 5-HT stands for 5-hydroxytryptamine, mCPP stands for meta-chlorophenylpiperazine, and DOI stands for 1-(2,5-dimethoxy-4-iodophenyl)isopropylamine.

To evaluate the affinity of various compounds of formula I for activity at the 5-HT_{2C} receptor, a CHO (Chinese Hamster Ovary) cell line transfected with the cDNA expressing the human 5-hydroxytryptamine-2C (h5-HT_{2C}) receptor was maintained in DMEM (Dulbecco's Modified Eagle Media) supplied with fetal calf serum, glutamine, and the markers: guaninephosphoribosyl transferase (GTP) and hypoxanthinethymidine (HT). The cells were allowed to grow to confluence in large culture dishes with intermediate changes of media and splitting. Upon reaching confluence, the cells were harvested by scraping. The harvested cells were suspended in half volume of fresh physiological phosphate buffered saline (PBS) solution and centrifuged at low speed (900×g). This operation was repeated once. The collected cells were then homogenized with a polytron at setting #7 for 15 sec in ten volumes of 50 mM Tris.HCl, pH 7.4 and 0.5 mM EDTA. The homogenate was centrifuged at 900×g for 15 min to remove nuclear particles and other cell debris. The pellet was discarded and the supernatant fluid recentrifuged at 40,000×g for 30 min. The resulting pellet was resuspended in a small volume of Tris.HCl buffer and the tissue protein content was determined in aliquots of 10-25 mL volumes. Bovine Serum Albumin (BSA) was used as the standard in the protein determination by the method of Lowry et al., (J. Biol. Chem., 193:265 (1951). The volume of the suspended cell membranes was adjusted with 50 mM Tris.HCl buffer containing 0.1% ascorbic acid, 10 mM pargyline and 4 mM CaCl₂ to give a tissue protein concentration of 1-2 mg per mL of suspension. The preparation membrane suspension (many times concentrated) was aliquoted in 1 mL volumes and stored at −70° C. until used in subsequent binding experiments.

Binding measurements were performed in a 96 well microtiter plate format, in a total volume of 200 μL. To each well was added: 60 μL of incubation buffer made in 50 mM Tris.HCl buffer, pH 7.4 and containing 4 mM CaCl₂; 20 μL of [¹²⁵I] DOI (S.A., 2200 Ci/mmol, NEN Life Science).

The dissociation constant, KD of [¹²⁵I] DOI at the human serotonin 5-HT_{2C} receptor was 0.4 nM by saturation binding with increasing concentrations of [¹²⁵I] DOI. The reaction was initiated by the final addition of 100 μL of tissue suspension containing 50 μg of receptor protein. Nonspecific binding is measured in the presence of 1 μM unlabeled DOI added in 20.0 μL volume. Test compounds were added in 20.0 μL. The mixture was incubated at room temperature for 60 min. The incubation was stopped by rapid filtration. The bound ligand-receptor complex was filtered off on a 96 well unifilter with a Packard ®Filtermate 196 Harvester. The bound complex caught on the filter disk was dried in a vacuum oven heated to 60° C. and the radioactivity measured by liquid scintillation with 40 μL Microscint-20 scintillant in a Packard TopCount® equipped with six (6) photomultiplier detectors.

Specific binding is defined as the total radioactivity bound less the amount bound in the presence of 1 μM unlabeled DOI. Binding in the presence of varying concentrations of test drugs is expressed as percent of specific binding in the absence of drug. These results are then plotted as log % bound vs log concentration of test drug. Non linear regression analysis of data points yields both the IC₅₀ and the Kᵢ values of test compounds with 95% confidence limits. Alternatively, a linear regression line of decline of data points is plotted, from which the IC₅₀ value can be read off the curve and the Kᵢ value determined by solving the following equation:

$${K}_{i} = \frac{{IC}_{50}}{1 + L / {K}_{D}}$$

where L is the concentration of the radioactive ligand used and the K_{D} is the dissociation constant of the ligand for the receptor, both expressed in nM.

The following Kᵢ's (95% confidence interval) are provided for various reference compounds in Table 2, below:

TABLE 2
Kᵢ Data for Reference Compounds

| Compound | Kᵢ | |
|---|---|---|
| Ritanserin | 2.0 (1.3-3.1) | nM |
| Ketanserin | 94.8 (70.7-127.0) | nM |
| Mianserin | 2.7 (1.9-3.8) | nM |
| Clozapine | 23.2 (16.0-34.0) | nM |
| Methiothepin | 4.6 (4.0-6.0) | nM |
| Methysergide | 6.3 (4.6-8.6) | nM |
| Loxapine | 33.0 (24.0-47.0) | nM |
| mCPP | 6.5 (4.8-9.0) | nM |
| DOI | 6.2 (4.9-8.0) | nM |

The ability of the compounds of formula I to produce an agonist response at brain 5-HT_{2C} was assessed by determining their effect on calcium mobilization using the following procedure: CHO cells stably expressing the human 5-HT_{2C} receptor were cultured in Dulbecco's modified Eagle's medium (DMEM) supplemented with 10% fetal bovine serum and non-essential amino acids. Cells were plated at a density of 40K cells/well in 96-well clear-bottom black-wall plates 24 hours prior to the evaluation of 5-HT_{2C} receptor-stimulated calcium mobilization. For calcium studies, cells were loaded with the calcium indicator dye Fluo-3-AM in Hank's buffered saline (HBS) for 60 minutes at 37° C. Cells were washed with HBS at room temperature and transferred to the fluorometric imaging plate reader (FLIPR, Molecular Devices, Sunnyvale, Calif.) for acquisition of calcium images. Excitation at 488 nm was achieved with an Argon ion laser and a 510-560 nm emission filter was used. Fluorescence images and relative intensities were captured at 1 second intervals and cells were stimulated by addition of agonist after 10 baseline measurements using the internal fluidics module of the FLIPR. An increase in fluorescence counts corresponds to an increase in intracellular calcium.

For the evaluation of agonist pharmacology the calcium changes in response to different concentrations of agonist were determined using a maximum minus minimum calculation of the raw fluorescence count data. Calcium changes were then expressed as a percentage of the response observed with a maximally effective concentration of 5-HT. IC₅₀ values were estimated by non-linear regression analysis of the log-concentration % maximum 5-HT response curves using the 4-parameter logistic function. In certain embodiments, compounds of the present invention provide an EC₅₀ of ≦about 1000 nM. In other embodiments, compounds of the present invention provide an EC₅₀ of ≦about 100 nM, in yet other embodiments ≦about 20 nM, in still other embodiments ≦about 5 nM, and certain embodiments ≦about 2 nM.

The following EC₅₀'s are provided for various reference compounds in Table 3, below

TABLE 3
EC₅₀ Data for Reference Compounds:

| Compound | EC₅₀ | |
|---|---|---|
| 5-HT EC₅₀ | 5 | nM |
| DOI EC₅₀ | 0.5 | nM |
| mCPP EC₅₀ | 5.4 | nM |
| SB242084 | 0.01 | nM |
| SB206553 | 13 | nM |

TABLE 4
5-HT2c Activity of Selected Compounds

| | | |
|---|---|---|
| Compound | 5-HT_{2C} Binding | 5-HT_{2C} Function |

| Number | Ki avg (nM) | EC₅₀ (nM) | E_{Max} (%) |
|---|---|---|---|
| Example 1 | 0.535 | 356 | 70 |
| Example 2 | 388 | | |
| Example 3 | 11.75 | | |

The compounds of this invention thus have affinity for and agonist or partial agonist activity at brain serotonin 5HT_{2C} receptors. They are therefore of interest for the treatment of the central nervous system conditions described previously herein.

### B. Assessment of Effectiveness of Compounds in Obesity Models

### Obesity Model A

To evaluate acute in vivo efficacy of various compounds, 7 weeks-old male C57BL/6J mice are obtained from The Jackson Laboratory (Bar Harbor, Me.) and 6 weeks-old lean Zucker rats are purchased from Charles River Laboratories (Wilmington, Mass.). Mice and rats are single housed in a temperature-controlled (25° C.) facility with a 12-h light/dark cycle. Animals are allowed normal chow diet (Rodent chow #5001, PharmaServ, Framingham, Mass.) and water ad libitum. After one week acclimation, animals are randomized to vehicle (saline) or treatment groups. Animals are fasted overnight (16 hrs) and orally dosed with vehicle or compounds. Thirty minutes after compound administration, animals were given a weighed amount of food, and food intake is recorded 30 minutes, 1 h, 2 h, 4 h, 7 h and 24 h after refeeding.

### Obesity Model B

To assess in vivo efficacy of various 5-HT_{2C} compounds on weight loss, 5 weeks-old male C57BL/6J-DIO mice are fed a high-fat high-sucrose diet (58 kcal % fat, 16.4 kcal % protein, 25.5 kcal % carbohydrate) for 11 weeks. 6 weeks-old male Zucker fa/fa rats purchased from Charles River Laboratories are also used. Mice and rats are single housed in a temperature-controlled (25° C.) facility with a 12-h light/dark cycle. Animals are allowed food and water ad libitum. After one week acclimation, animals are randomized to vehicle (saline) or treatment groups. Animals are orally dosed once daily for 14 days. Body weight, food consumption, and/or body composition (NMR) are recorded. Epidydimal adipose tissue is collected at the end of the study.

### C. Assessment of Effectiveness in Treatment of Pain

Compounds of formula I may be evaluated in accordance with the present invention to establish the extent of their effectiveness to treat pain, and may optionally be compared with other pain treatments.

A variety of methods have been established in the art to evaluate the effectiveness of compounds for relieving pain. See e.g., Bennett et al, Pain 33: 87-107, 1988; Chaplan et al, J. Neurosci. Methods 53:55-63, 1994; and Mosconi et al, Pain 64:37-57, 1996. Below is a specific description of one strategy that may be employed.

Procedure: Individually housed Spraque-Dawley rats are given free access to rat chow and water. A 12-h light/12-h dark cycle is put in effect (lights on from 6:00 am to 6:00 pm). Animal maintenance and research are conducted in accordance with the guidelines provided by the National Institutes of Health Committee on Laboratory Animal Resources. These subjects are used in the tests as set forth below.

### Test Method 1: Prostaglandin E₂-Induced Thermal Hypersensitivity.

The terminal 10 cm of the tail is placed into a thermos bottle containing water warmed to 38, 42, 46, 50, 54, or 58° C. The latency in seconds for the animal to remove the tail from the water is used as a measure of nociception. If the animal does not remove the tail within 20 sec, the experimenter removes the tail from the water and a maximum latency of 20 sec is recorded.

Following the assessment of baseline thermal sensitivity, thermal hypersensitivity is produced by a 50 μL injection of 0.1 mg prostaglandin E₂ (PGE₂) into the terminal 1 cm of the tail. Temperature-effect curves are generated before (baseline) and after (15, 30, 60, 90 and 120 min) the PGE₂ injection. Previous studies in other species (e.g., monkeys; Brandt et al., J. Pharmacol. Exper. Ther. 296:939, 2001) have demonstrated that PGE₂ produces a dose- and time-dependent thermal hypersensitivity that peaks 15 min after injection and dissipates after 2 hr.

Single compound studies. The ability of drugs to reverse PGE₂-induced thermal hypersensitivity is assessed using a single dose time-course procedure. Under this procedure, a single dose of the compound to be tested is administered intraperitoneally (IP), orally (PO) or intranasally (IN) 30 min before the injection of PGE₂. Tactile sensitivity is assessed 30 min after PGE₂ injection.
Combination compound studies. Combination studies with two or more potential pain treatment agents can be conducted. A minimally effective dose of a first agent, e.g., morphine is administered alone and in combination with ineffective doses of one or more compounds of formula I in the thermal warm-water tail withdrawal assay. Compounds are administered IP at the same time 30 min before testing.

Combination studies can also be conducted in the PGE₂-induced thermal hypersensitivity assay. For example, a dose of morphine that completely reverses thermal hypersensitivity (i.e., return to baseline) can be administered alone and in combination with doses of one or more compounds of formula I in the PGE₂-induced thermal warm-water tail withdrawal assay. Compounds are administered IP at the same time as PGE₂, which is administered 30 min before testing.

Test Method 1 Data Analysis The temperature that produced a half-maximal increase in the tail-withdrawal latency (i.e., T₁₀) is calculated from each temperature-effect curve. The T₁₀ is determined by interpolation from a line drawn between the point above and the point below 10 sec on the temperature-effect curve. For these studies, thermal hypersensitivity is defined as a leftward shift in the temperature-effect curve and a decrease in the T₁₀ value. Reversal of thermal hypersensitivity is defined as a return to baseline of the temperature-effect curve and the T₁₀ value and is calculated according to the following equation:

$$\% M P E = \frac{( {T}_{10}^{drug + PGE 2} ) - ( {T}_{10}^{PGE 2} )}{( {T}_{10}^{baseline} ) - ( {T}_{10}^{PGE 2} )} \times 100$$

in which T₁₀^{drug+PGE2} is the T₁₀ after a drug in combination with PGE₂, T₁₀^{PGE2} is the T₁₀ after PGE₂ alone, and T₁₀^{baseline} is the T₁₀ under control conditions. A % MPE value of 100 indicates a complete return to the baseline thermal sensitivity observed without the PGE₂ injection. A value of greater than 100% indicates that the compound tested reduced thermal sensitivity more than the baseline thermal sensitivity without the PGE₂ injection.

### Test Method 2: Chronic Constriction Injury

Rats are anesthetized with 3.5% halothane in O₂ at 1 L/min and maintained with 1.5% halothane in O₂ during surgery. A modified chronic sciatic nerve constriction injury (Mosconi & Kruger, 1996; Bennett & Xie, 1988) is produced by a cutaneous incision and a blunt dissection through the biceps femoris to expose the sciatic nerve. A PE 90 Polyethylene tubing (Intramedic, Clay Adams; Becton Dickinson Co.) cuff (2 mm length) is placed around the sciatic nerve at the level of the mid-thigh. The wound is closed in layers using 4-0 silk suture and wound clips. Testing is conducted 6-10 days after surgery.

Animals are placed in elevated wire cages and allowed 45-60 minutes to acclimate to the testing room. Baseline tactile sensitivity is assessed using a series of calibrated von Frey monofilaments (Stoelting; Wood Dale, Ill.) 0-3 days before surgery. Von Frey monofilaments are applied to the mid-plantar hind paw in sequential ascending or descending order, as necessary, to hover as closely as possible to the threshold of responses. The threshold is indicated by the lowest force that evoked a brisk withdrawal response to the stimuli. Thus, a withdrawal response leads to the presentation of the next lighter stimulus and the lack of a withdrawal response leads to the presentation of the next stronger stimulus. Rats with baseline thresholds <4 g force are excluded from the study. Approximately one week following CCI surgery, tactile sensitivities are reassessed and animals that exhibit motor deficiency (i.e. paw dragging) or failure to exhibit subsequent tactile hypersensitivity (threshold ≧10 g) are excluded from further testing. Under cumulative dosing conditions, compounds are administered IP every 30 minutes with the cumulative dose increasing in ½ log unit increments. Tactile hypersensitivity is assessed 20-30 minutes following each drug administration.

Test Method 2 Data Analysis. The 50% threshold values (in gm force) estimated by the Dixon non-parametric test (Chaplan et al, 1994) are calculated and fifteen-grams of force is used as the maximal force. Dose-effect curves are generated for each experimental condition for each rat. Individual tactile hypersensitivity threshold values are averaged to provide a mean (±1 SEM). Reversal of tactile hypersensitivity was defined as a return to baseline tactile sensitivity and was calculated according to the following equation:

$$\% Reversal = \frac{( 50 {\%}^{drug + CCI} ) - ( 50 {\%}^{CCI} )}{( 50 {\%}^{baseline} ) - ( 50 {\%}^{CCI} )} \times 100$$

in which 50%^{drug+CCI} is the 50% value after compound in animals approximately one week after CCI surgery, 50%^{CCI} is the 50% value approximately one week after CCI surgery alone, and 50%^{baseline} is the 50% value before CCI surgery. Maximal effect of 100% reversal represents a return to the mean pre-operative threshold value for subjects in that experimental condition.

### Test Method 3: Scheduled-Controlled Responding.

Rats are trained under a multiple-cycle procedure during experimental sessions conducted five days each week. Each training cycle consists of a 10-min pretreatment period followed by a 10-min response period. During the pretreatment period, stimulus lights are not illuminated, and responding has no scheduled consequences. During the response period, the left or right stimulus lights are illuminated (counterbalanced among subjects), the response lever is extended and subjects can respond under a fixed ratio 30 schedule of food presentation. Training sessions consist of 3 consecutive cycles. Testing sessions are identical to training sessions except that a single dose of drug is administered at the start of the first cycle.

Test Method 3 Data analysis. Operant response rates from individual animals are averaged for the three cycles during test sessions and are converted to percent of control response rates using the average rate from the previous training day as the control value (i.e., average of three cycles). Data are presented as the mean (±1 SEM) response rate as a percent of control. Thus, for example, a test value of 100% would indicate the response rate after administration of the compound to be tested is the same as the control response rate and there is no adverse effect of the compound tested.

### Test Method 4: Assessment of Effectiveness in Tactile Allodynia Model

Compound: Test compounds are dissolved in sterile saline and gabapentin is suspended in 2% Tween 80 in 0.5% methylcellulose and sterile water. All compounds are administered intraperitoneally (i.p.).
Subjects: Male Sprague-Dawley rats (125-150 g, Harlan; Indianapolis, Ind.) are individually housed on bedding. For all studies animals are maintained in climate-controlled rooms on a 12-hour light/dark cycle (lights on at 0630) with food and water available ad libitum.
Surgery: All surgical procedures are performed under 4% isoflurane/O₂ anesthesia, delivered via nose cone and maintained at 2.5% for the duration of the surgery.
L5 Spinal Nerve Ligation (SNL): Surgery is performed as previously described (Kim and Chung) with the exception that nerve injury is produced by tight ligation of the left L5 spinal nerve.
Assessment of Tactile Allodynia (Tactile Sensitivity): Tactile thresholds are assessed using a series of calibrated von Frey monofilaments (Stoelting; Wood Dale, Ill.). The threshold that produced a 50% likelihood of a withdrawal is determined using the up-down method, as previously described (Chaplan et al., 1994). Animals are placed in elevated wire cages and allowed 45-60 minutes to acclimate to the testing room. Von Frey monofilaments are applied to the mid-plantar left hind paw in sequential ascending or descending order, as necessary, to hover as closely as possible to the threshold of responses. The lowest force that evokes a brisk withdrawal response to the stimuli determined the pain threshold. Tactile thresholds are determined on the day prior to surgery and rats with baseline thresholds <10 g force are excluded from studies. Three weeks after SNL surgery tactile thresholds are reassessed and animals that fail to exhibit subsequent tactile allodynia (threshold ≧5 g) are excluded from further testing. Subjects are pseudo-randomly divided into test groups (n=8−10) so that average baseline and post-surgery sensitivities are similar among groups. Rats are administered a test compound (3, 10 or 17.8, i.p.), gabapentin (100 mg/kg, i.p., positive control) or vehicle and tactile thresholds are assessed up to 60, 180 and 300 minutes after dosing.
Analysis of Results: Statistical analysis is done using a repeated measures analysis of variance (ANOVA) using a customized SAS-excel application (SAS Institute, Cary, N.C.). Significant main effects are analyzed further by subsequent least significant difference analysis. The criterion for significant differences is p<0.05. Reversal of tactile allodynia is calculated according to the following equation:

$$\% Reversal = \frac{( 50 \% {threshold}^{drug + post surgery} ) - ( 50 \% {treshold}^{post surgery} )}{( 50 \% {threshold}^{pre surgery} ) - ( 50 \% {threshold}^{post surgery} )} \times 100$$

In which 50% threshold^{drug+post surgery} is the 50% threshold in g force after drug in nerve injured subjects, 50% threshold^{post surgery} is the 50% threshold in g force in nerve injured subjects, and 50% threshold^{pre surgery} is the 50% threshold in g force before nerve injury. Maximal effect of 100% reversal represents a return to the mean pre-operative threshold value for subjects in that experimental condition.

### Test Method 5:Assessment of Effectiveness in Chronic Inflammatory Pain

### Compounds:

Test compounds are dissolved in sterile saline and administered intraperitoneally (i.p.). Celecoxib was used as a positive control and is suspended in 2% Tween 80 in 0.5% methylcellulose and administered orally (p.o.).

Subjects: Male Sprague-Dawley rats (125-150 g, Harlan; Indianapolis, Ind.) are housed 3/cage on bedding and. animals are maintained in climate-controlled rooms on a 12-hour light/dark cycle (lights on at 0630) with food and water available ad libitum.
Freund's complete adjuvant (FCA) of mechanical hyperalgesia: The hind paw withdrawal thresholds (PWTs) to a noxious mechanical stimulus are determined using an analgesimeter (model 7200; Ugo Basile). Cutoff was set at 250 g, and the endpoint taken is complete paw withdrawal. PWT is determined once for each rat at each time point (n=10/group). Baseline PWT is determined, and the rats were anesthetized with isofluorane (2% in oxygen) and received an intraplantar injection of 50% FCA (50 μl, diluted in saline) to the left hind paw. Twenty-four hours after FCA injection, pre-drug PWTs were measured, and the rats are administered vehicle or compound and assessed on PWTs 1, 3, 5, and 24 hours post-drug administration.
Analysis of Results: Statistical analysis is done using a one way analysis of variance (ANOVA) using a customized SAS-excel application (SAS Institute, Cary, N.C.). Significant main effects are analyzed further by subsequent least significant difference analysis. The criterion for significant differences is p<0.05 from vehicle-treated FCA rats. Data is presented as percent reversal according to the following equation: percent reversal=[(post-dose threshold)—pre-dose threshold))/(baseline threshold-pre-dose threshold)]×100.

### D. Assessment of Effectiveness in Treatment of Depression

Effectiveness of compounds of the present invention may be determined by the tail suspension test. While not a direct model of depression, the tail suspension test is an assay that can evaluate antidepressant-like effects of drugs. Clinically effective drugs such as Prozac (fluoxetine) are effective in this assay. Specifically, they decrease the amount of time the mice spend immobile after being hung upside down by their tails during the test. It is impossible to determine if a mouse is indeed depressed. However, the fact that clinically effective antidepressants reduce immobility lends predictive validity to the model.

Male Swiss Webster mice (Charles River) weighing 25-35 g are housed in groups of five per cage in an AALAC-accredited facility that is maintained on a 12-h light dark cycle (lights on at 0600 h) and have free access to food and water. Experimental groups consist of 12 mice, randomly assigned to treatment groups. Experiments are performed between 9:00 AM and noon in accordance to the Guide for the Care and Use of Laboratory Animals as adopted and promulgated by the National Institutes of Health (Pub. 85-23, 1985).

Solutions of test compounds are dissolved in distilled water. Compounds are injected i.p. at a volume of 10 ml/kg body weight. Combination treatments are cotreated, 30 minutes prior to the test.

The procedure described herein is substantially similar to that described by Stem et al. (1985). 30 minutes following treatment, the mice are suspended upside down by the tail using adhesive laboratory tape (VWR International), to a flat metal bar connected to a strain gauge within a tail suspension chamber (Med Associates). The time spent immobile during a 6-minute test session is automatically recorded. 8 mice are simultaneously tested within separate chambers. Data collected are expressed as a mean of immobility time and statistical analysis is performed using a one-way ANOVA with least significant difference (LSD) post-hoc test.

The entire disclosure of each patent, patent application, and publication cited or described in this document is hereby incorporated by reference.

While we have presented a number of embodiments of this invention, it is apparent that our basic construction can be altered to provide other embodiments which utilize the compounds and methods of this invention. Therefore, it will be appreciated that the scope of this invention is to be defined by the appended claims rather than by the specific embodiments which have been represented by way of example.

1. A compound of formula I: 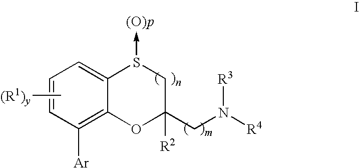
or a pharmaceutically acceptable salt thereof, wherein:
p is 0, 1, or 2;
m is 1 or 2;
n is 0 or 1;
Ar is phenyl, an 8-10 membered bicyclic partially unsaturated or aryl carbocyclic ring, a 5-6 membered monocyclic heteroaryl having 1-4 heteroatoms independently selected from nitrogen, oxygen, or sulfur, or an 8-10 membered bicyclic partially unsaturated or heteroaryl ring having 1-5 heteroatoms independently selected from nitrogen, oxygen, or sulfur, wherein Ar is optionally substituted with one or more R^{x} groups;
each R^{x} is independently selected from —R, —CN, halogen, —OR, —O(C₁₋₆ haloalkyl), —C(O)NH₂, —C(O)OR, C₁₋₆ haloalkyl, —NHC(O)R, —SO₂R, or —NHSO₂R;
y is 0-3;
each R¹ is independently —R, —CN, halogen, —OR, —O(C₁₋₆ haloalkyl), —C(O)NH₂, —C(O)OR, C₁₋₆ haloalkyl, —NHC(O)R, —SO₂R, or —NHSO₂R;
each R is independently hydrogen or C₁₋₆ aliphatic;
each of R², R³ and R⁴ is independently R or C₁₋₆ haloalkyl.

2. The compound according to claim 1, wherein said compound is of formula Ia: 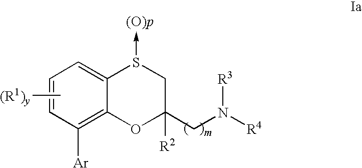
or a pharmaceutically acceptable salt thereof.

3. The compound according to claim 1, wherein said compound is of formula Ib: 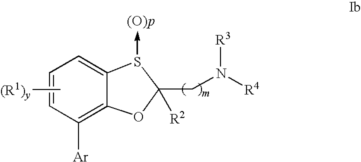
or a pharmaceutically acceptable salt thereof.

4. The compound according to claim 2, wherein said compound has the formula IIa or IIb: 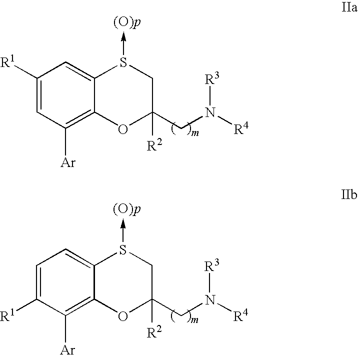
or a pharmaceutically acceptable salt thereof.

5. The compound according to claim 3, wherein said compound is of formula IIc or IId: 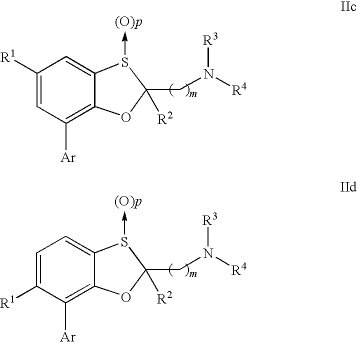
or a pharmaceutically acceptable salt thereof.

6. The compound according to any one of claims 1 to 5, wherein Ar is phenyl, an 8-10 membered bicyclic partially unsaturated or aryl carbocyclic ring, or a 5-6 membered monocyclic heteroaryl having 1-4 heteroatoms independently selected from nitrogen, oxygen, or sulfur.

7. The compound according to any one of claims 1 to 5, wherein Ar is pyridyl, pyrimidinyl, thienyl, or furanyl, or phenyl optionally substituted with one or more R^{x} groups.

8. The compound according to claim 2, wherein said compound is of formula IIIa or IIIc: 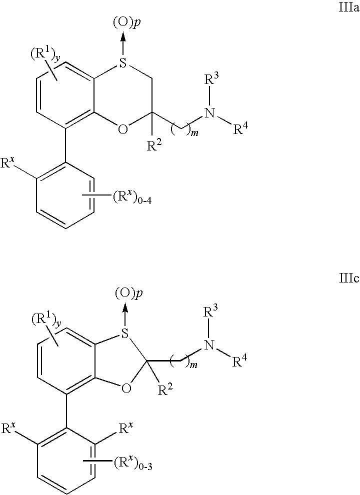
or a pharmaceutically acceptable salt thereof.

9. The compound according to claim 3, wherein said compound is of formula IIIb or IIId: 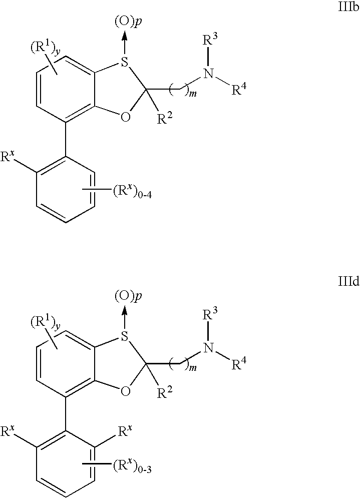
or a pharmaceutically acceptable salt thereof.

10. The compound according to claim 7, wherein each R^{x} is independently selected from —R, —CN, halogen, —OR, OCF₃, or —CF₃.

11. The compound according to claim 1, wherein each R¹ is independently —R, —CN, halogen, —OR, —OCF₃, or —CF₃.

12. The compound according to claim 1, wherein R² is hydrogen or methyl.

13. The compound according to claim 1, wherein each of R³ and R⁴ is independently hydrogen, methyl, ethyl, cyclopropyl, cyclopropylmethyl, n-propyl, allyl, or cyclobutyl.

14. The compound according to claim 1, wherein:
each R¹ is independently —R, —CN, halogen, —OR, —OCF₃, or —CF₃;
R² is hydrogen or methyl;
Ar is pyridyl, pyrimidinyl, thienyl, furanyl, or phenyl optionally substituted with one or more R^{x} groups;
each R^{x} is independently selected from —R, —CN, halogen, —OR, OCF₃, or —CF₃; and
each of R³ and R⁴ is independently hydrogen, methyl, ethyl, cyclopropyl, cyclopropylmethyl, n-propyl, allyl, or cyclobutyl.

15. The compound according to claim 1, wherein Ar is selected from:

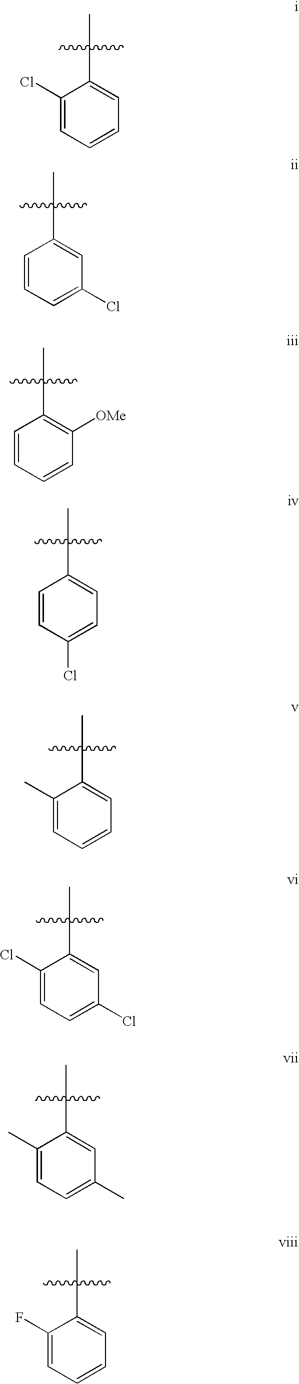

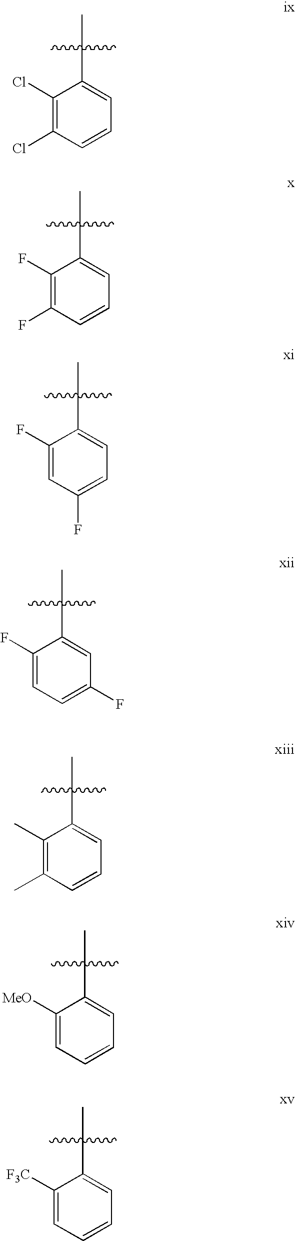

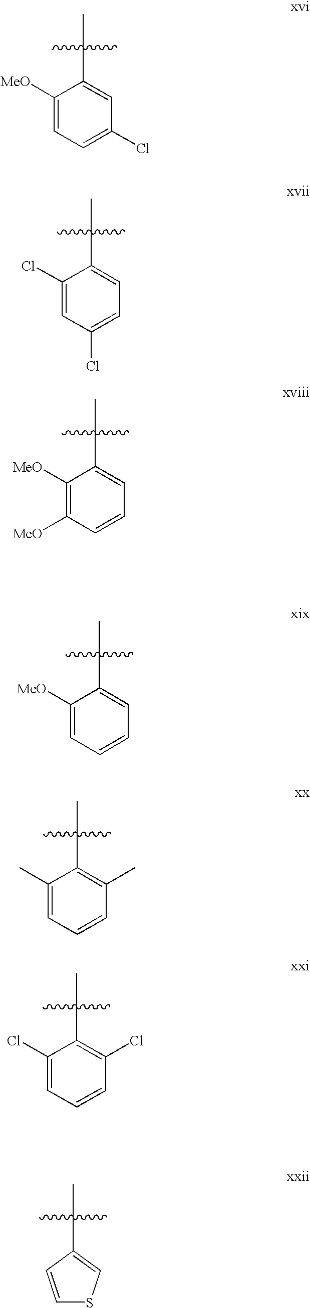

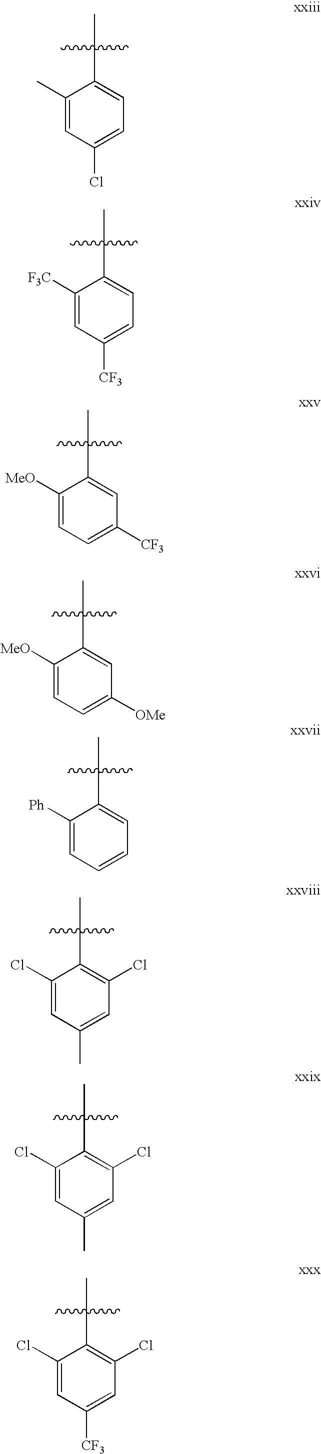

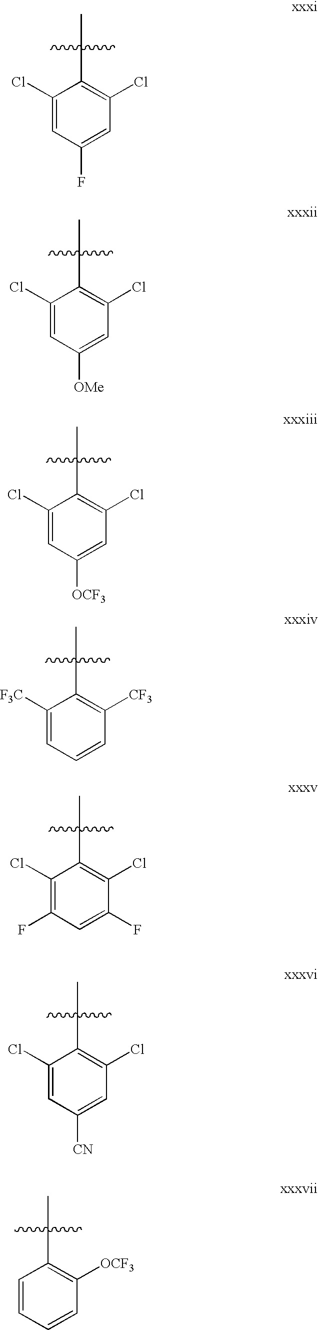

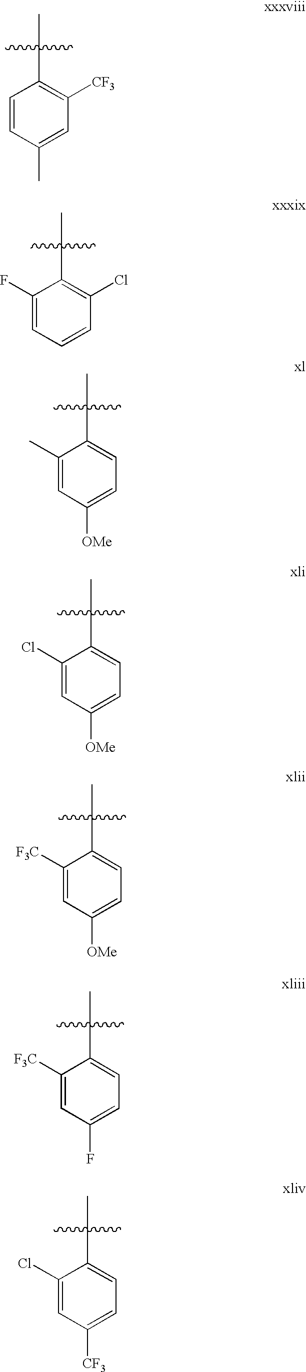

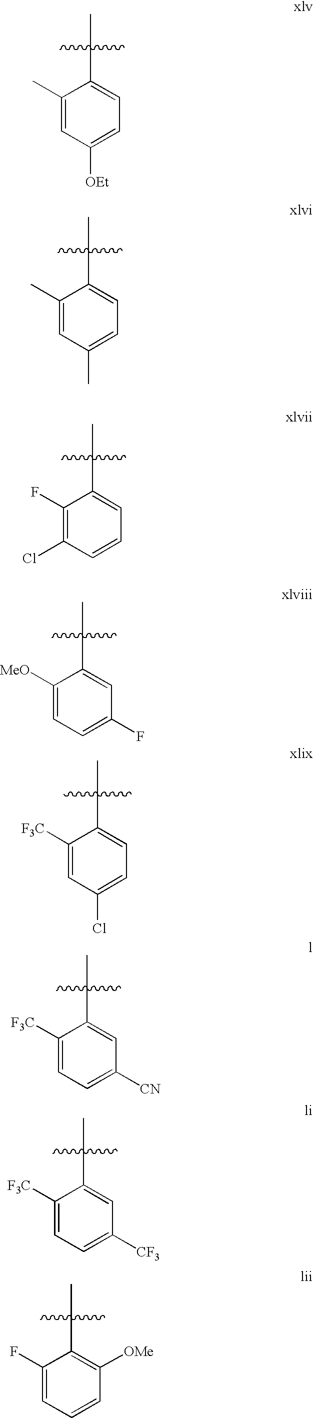

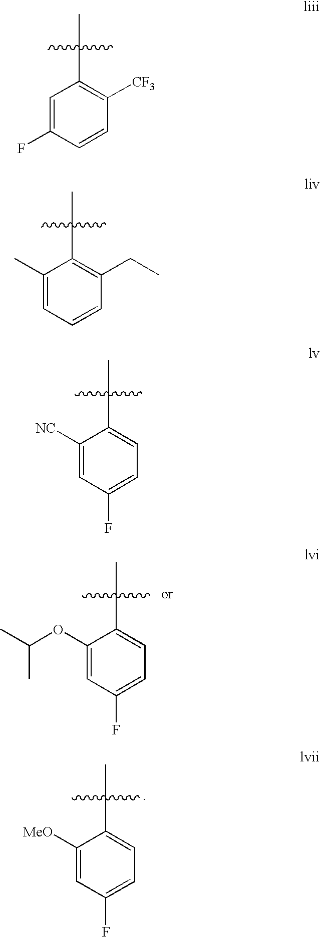

16. The compound according to claim 1, wherein said compound is selected from:
C-[8-(2,6-dichloro-phenyl)-6-fluoro-2,3-dihydro-benzo[1,4]oxathiin-2-yl]-methylamine;
C-[8-(2,6-dichloro-phenyl)-6-fluoro-4,4-dioxo-3,4-dihydro-2H-4lambda-6-benzo[1,4]oxathiin-2-yl]-methylamine hydrochloride salt; and
C-[8-(2,6-dichloro-phenyl)-6-fluoro-4-oxo-3,4-dihydro-2H-4lambda-4-benzo[1,4]oxathiin-2-yl]-methylamine;
1-[7-(2,6-dichlorophenyl)-5-fluoro-1,3-benzoxathiol-2-yl]methanamine;
1-{6-fluoro-8-[2-(trifluoromethyl)phenyl]-2,3-dihydro-1,4-benzoxathiin-2-yl}methanamine;
1-[7-(2,6-dichlorophenyl)-5-fluoro-3,3-dioxido-1,3-benzoxathiol-2-yl]methanamine;
1-{5-fluoro-7-[2-(trifluoromethyl)phenyl]-1,3-benzoxathiol-2-yl}methanamine; and
1-[8-(2-chlorophenyl)-6-fluoro-2,3-dihydro-1,4-benzoxathiin-2-yl]methanamine,
or a pharmaceutically acceptable salt, enantiomer or racemate thereof.

17. A composition comprising a compound as claimed in claim 1, and one or more pharmaceutically acceptable carriers, diluents, or excipients.

18. The composition of claim 17, further comprising an additional pharmaceutical agent selected from an anti-psychotic agent, an antidepressive agent, an anti-obesity agent, an agent useful in the modulation of bladder activity, an opioid antagonist, an agent for treating ADD or ADHD, a cognitive improvement agent, an agent for treating sexual dysfunction, or a pain relieving agent.

19. A method for treating a condition selected from at least one of psychotic disorder, an anxiety disorder, a bipolar disorder, a depressive disorder, premenstrual syndrome (PMS), premenstrual dysphoric disorder (PMDD), an eating disorder, a bladder control disorder, substance abuse or substance dependence, a cognition disorder, ADD or ADHD, an impulsivity disorder, an addictive disorder, male or female sexual dysfunction, pain, a motion or motor disorder, Parkinson's disease epilepsy, migraine, chronic fatigue syndrome, anorexia nervosa, a sleep disorder, mutism, or one or more central nervous system deficiencies in a patient, comprising administering to the patient a therapeutically effective amount of a compound according to claim 1 or a composition thereof.

20. The method of claim 19 wherein the psychotic disorder is schizophrenia, paranoid type schizophrenia, disorganized type schizophrenia, catatonic type schizophrenia, undifferentiated type schizophrenia, a schizophreniform disorder, a schizoaffective disorder, a delusional disorder, substance-induced psychotic disorder, a psychotic disorder not otherwise specified; L-DOPA-induced psychosis; psychosis associated with Alzheimer's dementia; psychosis associated with Parkinson's disease; or psychosis associated with Lewy body disease

21. The method of claim 19, wherein the condition is bipolar disorder and is selected from bipolar I disorder, bipolar II disorder, cyclothymic disorder; bipolar mania, dementia, depression with psychotic features, or cycling between bipolar depression and bipolar mania.

22. The method of claim 19, wherein the depressive disorder is major depressive disorder, seasonal affective disorder, dysthymic disorder, substance-induced mood disorder, depressive disorder not otherwise specified, treatment resistant depression, major depressive episode.

23. The method of claim 22, further comprising administering to the patient an antidepressive agent selected from serotonin reuptake inhibitors (SRIs), norepinephrine reuptake inhibitors (NRIs), combined serotonin-norepinephrine reuptake inhibitors (SNRIs), monoamine oxidase inhibitors (MAOIs), reversible inhibitors of monoamine oxidase (RIMAs), phosphodiesterase-4 (PDE4) inhibitors, corticotropin releasing factor (CRF) antagonists, alpha.-adrenoreceptor antagonists, triple uptake inhibitors, melatonin agonists, super neurotransmitter uptake blockers (SNUBs), noradrenergic and specific serotonergic antidepressants (NaSSAs), or substance P/neurokinin receptor antagonists.

24. The method of claim 19, wherein the cognitive disorder is a learning disorder.

25. The method of claim 19, wherein the patient is treated for obesity.

26. The method of claim 19, wherein the patient is treated for ADD or ADHD.

27. The method of claim 19, wherein the substance abuse substance dependence is of a recreational substance, a pharmacologic agent, a tranquilizer, a stimulant, sedative, or illicit drug.

28. The method of claim 19, further comprising administering to the patient an additional pharmaceutical agent selected from an anti-psychotic agent, an antidepressive agent, an anti-obesity agent, an agent useful in the modulation of bladder activity, an opioid antagonist, an agent for treating ADD or ADHD, a cognitive improvement agent, an agent for treating sexual dysfunction, or a pain relieving agent.

29. A method for treating schizophrenia in a patient, comprising administering to the patient a therapeutically effective amount of a composition according to claim 17.

30. A method for treating obesity in a patient, comprising administering to the patient a therapeutically effective amount of a composition according to claim 17.

31. A method for treating bipolar disorder in a patient, comprising administering to the patient a therapeutically effective amount of a composition according to claim 17.

32. A method for treating depression in a patient, comprising administering to the patient a therapeutically effective amount of a composition according to claim 17.

33. (canceled)
